(19) United States
(12) Patent Application Publication
(10) Pub. No.: US 2010/0279273 A1
(43) Pub. Date: Nov. 4, 2010

(54) NUCLEIC ACID SEQUENCES FOR THE AMPLIFICATION AND DETECTION OF RESPIRATORY VIRUSES

(75) Inventors: Michel G. Bergeron, Quebec (CA); Johanne Frenette, Quebec (CA); Maurice Boissinot, St-Augustin-de-Desmaures (CA); Eric Leblanc, Vancouver (CA); Guy Boivin, Sainte-Foy (CA); Natasha Dionne, Quebec (CA)

Correspondence Address: CHOATE, HALL & STEWART LLP, TWO INTERNATIONAL PLACE, BOSTON, MA, 02110, US

(73) Assignee: UNIVERSITE LAVAL, Quebec (CA)

(21) Appl. No.: 12/669,272

(22) PCT Filed: Jul. 17, 2008
(86) PCT No.: PCT/CA08/01315

§ 371 Date: Jun. 18, 2010

Related U.S. Application Data
(60) Provisional application No. 60/929,902, filed on Jul. 17, 2007.

(51) Int. Cl. C12Q 1/70; C07H 21/00; C40B 40/06

(52) U.S. Cl. 435/5; 536/25.6; 506/16

(57) ABSTRACT

The present invention relates to methods of detection, as well as assays, reagents and kits for the specific detection of 15 clinically important respiratory viruses including influenza A and B viruses, human respiratory syncytial viruses, human metapneumoviruses, human enteroviruses, all serotypes of rhinoviruses, 7 serotypes of adenoviruses, parainfluenza viruses types 1, 2, 3, and 4, as well as coronaviruses NL, 229E, OC43, and SARS-CoV. The present invention allows for the detection of each of these respiratory viruses in a single assay.

### FIELD OF THE INVENTION

The present invention relates to methods of detection of respiratory viruses as well as assays, reagents and kits for their specific detection.

### BACKGROUND OF THE INVENTION

The economical and health burden of viral respiratory tract infections (VRTIs) is appalling. Respiratory tract infections cause nearly half of the deaths due to infectious diseases in the USA (Wei and Norwood, 2001, Obstet. Gynecol. Clin. North Am. 28:283-304), influenza infection being the sixth leading cause of death (Maher et al., 2006, Am. J. Infect. Control 34:E107). Every year, approximately 200,000 Americans are hospitalized and more than 36,000 die from influenza and influenza-related complications. Over the past 100 years, this virus has taken its toll during the yearly epidemics and the occasional pandemics. In fact, influenza was responsible for more than 20 million deaths during the 1918 pandemics only.

More rapid diagnostic methods will provide clinicians with crucial information that should help to improve the management of infections associated with respiratory viruses and should contribute to reduce health care costs.

Genetic-based detection of virus has greatly progressed over the past decades. However, one difficulty associated with genetic-based assays is the variability of the genetic material from one strain or species to the other. As such, a suitable assay would require assaying for the presence of multiple pathogens or even for the presence of several strains of pathogens in order to make a valid diagnosis.

One way to address these difficulties is to design assays for the specific detection of multiple pathogens in the same assay based on the amplification of the pathogen's genetic material by using for example, polymerase chain reaction (PCR). However, such types of assays suffer from drawbacks related to the lack of specificity, i.e., cross-reactivity of primers and/or probes with genetic material from undesired strains or pathogens, as well as the lack of sensitivity and reproducibility. In fact, multiplexing PCR primers is difficult as the presence of several pairs of primers together in the same container increases the probabilities of mispairing and the formation of non-specific amplification products such as primer-dimers.

Conditions required to obtain probe set combinations presenting the essential characteristics of specificity, sensitivity and uniformity have recently been reviewed (Loy and Bodrossy, 2006, Clin. Chim. Acta 263:106-119). Based on their analysis, the ideal properties for highly specific recognition, efficient binding and uniform thermodynamic behaviour represent conflicting goals difficult to achieve in practice. They propose to use careful design rules but admit that the predictive value of these rules is known to be unreliable for solid support hybridization and experimental validation of the probe combinations is required. Another approach they suggest is to add redundancy in the probe combination strategy. However, adding more probes increases cost and complexity while limiting miniaturization and parallelization capacity.

Briese et al. has developed an assay that appears to detect 22 VRTIs using a combination of PCR amplification and mass spectrometry technology (Briese et al., 2005, Emerg. Infect. Dis. 11:310-313). The sensitivity attained using this technology was 500-1000 RNA copies of target genes. Other groups developed TaqMan multiplex PCR assays for the simultaneous detection of 7, 9, or 12 respiratory viruses, all included in the present invention (Gröndahl et al., 1999, J. Clin. Microbiol. 37:1-7; Syrmis et al., 2004, J. Mol. Diagn. 6:125-131; Templeton et al., 2004, J. Clin. Microb. 42:1564-1569; Gunson et al., 2005, J. Clin. Virol. 33:341-344; Bellau-Pujol et al., 2005, J. Virol. Methods 126:53-63). Two patents have been issued for the detection of respiratory viruses by multiplex PCR. For these two inventions, specific detection of the target respiratory viruses is performed using either enzyme hybridization assay (U.S. Pat. No. 6,015,664) or microarrays (U.S. Pat. No. 6,881,835).

Our analysis of primer sequences described in the above mentioned references suggest that ubiquity is incomplete since they do not appear to detect all viral strains of a target species.

There is thus a need for improved reagents and assays allowing the specific and sensitive detection of the most clinically important respiratory tract viral pathogens.

The present invention seeks to meet these and other needs.

### SUMMARY OF THE INVENTION

The present invention relates to methods of detection of respiratory viruses as well as reagents, assays and kits for the specific detection of clinically relevant RNA and DNA respiratory viruses.

The present invention more particularly provides methods, reagents, assays and kits for the specific and sensitive detection of 15 of the most clinically important respiratory tract viral pathogens including (i) the influenza A and B viruses, (ii) the human respiratory syncytial virus (RSV), (iii) the human metapneumovirus (hMPV), (iv) the human enteroviruses and rhinoviruses (all known serotypes), (v) the parainfluenza virus types 1, 2, 3, and 4, (vi) the coronaviruses NL, 229E, OC43, and SARS-CoV (associated with the Severe Acute Respiratory Syndrome (SARS)), as well as (vii) all adenoviruses serotypes associated with VRTIs.

The methods of detection may be carried out by amplification of the genetic material with virus-specific oligonucleotides, by hybridization of the genetic material with virus-specific oligonucleotides or by a combination of amplification and hybridization.

A significant advantage of the present invention is that the amplification step may be performed under similar or uniform amplification conditions for each respiratory virus species. As such, amplification of each respiratory virus species may be performed simultaneously. In accordance with an embodiment of the invention, detection of the respiratory viruses may be performed in parallel.

Another significant advantage of the invention is that hybridization may also be performed under similar of uniform hybridization conditions.

Furthermore, detection of the genetic material may also be performed under similar or uniform detection conditions.

Thus, aspects of the invention relates to methods for detecting and/or identifying a respiratory virus which may include the steps of contacting a sample comprising or suspected of comprising a genetic material originating from the respiratory virus and;—the oligonucleotide or combination of oligonucleotides under suitable conditions of hybridization, amplification and/or detection.

The methods, reagents, assays and/or kits may be based on the specific detection of (i) the matrix gene of influenza A virus (ii) the matrix gene of influenza B virus, (iii) the nucleocapsid gene of human respiratory syncytial virus, (iv) the nucleocapsid gene of the human metapneumovirus, (v) the 5′-non-coding region of the human enteroviruses, (vi) the 5′-non-coding region of the rhinoviruses (all known serotypes), (vii) the fusion gene of each of the parainfluenza virus types 1, 2, 3, and 4, (viii) the matrix gene of the coronavirus OC43, (ix) the polymerase gene of each of the coronaviruses NL, 229E, and SARS-CoV, as well as (x) the hexon region of adenoviruses serotypes associated with VRTIs.

Since similar amplification conditions may be used, one advantage of the present invention is that amplification of several respiratory virus species may be performed in the same vial or container. For example, the human enterovirus, the rhinovirus, the human respiratory syncytial virus and the human metapneumovirus may be performed in the same vial or container. Amplification of influenza A, parainfluenza type 1, parainfluenza type 2 and parainfluenza type 3 may be performed in the same vial or container. Amplification of the coronaviruses SARS-CoV, 229E, NL and OC43 may also be performed in the same vial or container. Amplification of adenovirus, influenza B and parainfluenza type 4 may also be performed in the same vial or container. Of course, if desired, the detection of the respiratory viruses may be performed separately (i.e., in separate or distinct test tubes and/or in separate experiments).

Aspects of the invention relate to reagents capable of specific binding to the genetic material of the respiratory viruses including primers, probes, combinations of primers, combination of probes or combination of primers and probes.

To the best of the Applicant's knowledge, the combinations of primers and/or probes presented herein have not been previously described. Indeed, the other assays presented in the above-mentioned studies either do not aim at detecting the same genetic targets or use different amplification primers/probes.

It is believed that the present invention may be useful not only to detect the known target viral species but may also be useful for the detection and identification of yet undiscovered species belonging to the targeted genera.

### DETAILED DESCRIPTION OF THE INVENTION

As know in the art, genetic material may be detected by amplification and/or hybridization using primers or probes optimally designed for their specific detection. The challenge related to the amplification of genetic material from viruses is that some pathogens have their genetic material in the form of single-stranded or double-stranded RNA, single-stranded or double-stranded DNA or even RNA/DNA chimera. The characteristics of the genetic material of each pathogens sought to be detected should be kept in mind for the design of specific primers and/or probes.

Nevertheless, it is well known in the art that RNA can be converted into DNA by the reverse transcriptase (RT) enzyme. Alternatively, DNA can be converted into RNA when, for example, an appropriate promoter (e.g. RNA polymerase promoter) and/or other regulatory elements are in operative connection with it. Therefore, the nucleic acid template (target) used to carry out the present invention may be either DNA (e.g., a genomic fragment, a synthetic fragment, a restriction fragment, etc.) or RNA, either single-stranded or double-stranded.

The nucleic acid target (genome, gene or gene fragment (e.g., a restriction fragment) from a respiratory virus) may be in a purified, unpurified form or in an isolated form. The nucleic acid target may be contained within a sample including for example, a biological specimen obtained from a patient, a sample obtained from the environment (soil, objects, etc.), a microbial or tissue culture, a cell line, a preparation of pure or substantially pure pathogens or pathogen mixture, etc. In accordance with the present invention, the sample may be obtained from patient having or suspected of having symptoms of a respiratory infection.

Oligonucleotide Primers and Probes Design and Synthesis

Due to the lack of understanding of the hybridization behaviour of oligonucleotide primers and/or probes which are affected by immobilization to solid support, steric hindrance, dissociation of mixed targets, etc. obtaining specific and sensitive probe sequences is a challenge that needs to be carefully addressed. For example, it appears that nonequilibrium thermal dissociation model cannot efficiently predict which primer or probe sequence will interact perfectly, specifically with its complementary sequence and under which stringency conditions (Pozhitkov et al., 2007 Nucl. Acids Res. 35:e70).

In the present study, multiple sequence alignments have been generated from sequence data of public databases for target respiratory viral species as well as of other related respiratory viral species, including viral sequences from animal sources and also from new sequence data generated by the Applicant by sequencing selected target genes from clinical specimens collected from patients having VRTIs (Table 1).

Accession numbers of public sequences used in the selection of primers and probes of the present invention are listed in Table 1. Visual inspection of the alignments generated allowed the selection of conserved sequence within each target viral species while allowing to discriminate sequences from other viral species, particularly those which are closely related. This strategy led to the selection of a total of 2145 sequences for the chosen target genes (matrix, nucleocapsid, fusion, hexon, polymerase, and the 5′-non coding region) which led to the generation of PCR/RT-PCR primers and probes specific for each target virus.

As part of the design strategy, all oligonucleotides (probes for hybridization and primers for DNA amplification by PCR and reverse transcriptase PCR (RT-PCR)) were evaluated for their suitability for hybridization or PCR/RT-PCR amplification by computer analysis using commercial programs (i.e. the Genetics Computer Group (GCG) programs, the primer analysis software Oligo™ 6.22). The suitability of the PCR primer pairs was also evaluated prior to their synthesis by verifying the absence of unwanted features such as long stretches of one nucleotide and a high proportion of G or C residues at the 3′ end.

Primers and probes of the present invention therefore allow for the specific detection of representative strains of each targeted viral species or genus belonging to (i) the influenza A and B viruses, (ii) the human respiratory syncytial virus (RSV), (iii) the human metapneumovirus (hMPV), (iv) the human enteroviruses and rhinoviruses (all known serotypes), (v) the parainfluenza virus types 1, 2, 3, and 4, (vi) the coronaviruses NL, 229E, OC43, and SARS-CoV, as well as (vii) adenoviruses serotypes associated with VRTIs (all 7 serotypes). Combinations of primers and probes were tested and the most efficient combinations were selected.

Although the sequences from selected target genes are available from public databases and, in a few cases, have been used to develop nucleic acid-based assays for detection of these viruses, the combination of primers and/or probes sequences of the present invention present unique advantages over those found in the literature in that they were designed to detect most strains of each target viral species and not only one or a few strains. The reagents and assays of the present invention may thus improve current nucleic acid-based assays for the diagnosis of respiratory viruses as they allow the simultaneous detection and/or identification of most known respiratory viral species including major epidemic strains around the world.

The present invention more particularly provides reagents, assays and kits for the detection of respiratory viruses using (i) the matrix gene of influenza A virus (ii) the matrix gene of influenza B virus, (iii) the nucleocapsid gene of human respiratory syncytial virus, (iv) the nucleocapsid gene of the human metapneumovirus, (v) the 5′-non-coding region of the human enteroviruses, (vi) the 5′-non-coding region of the rhinoviruses (all known serotypes), (vii) the fusion gene of each of the parainfluenza virus types 1, 2, 3, and 4, (viii) the matrix gene of the coronavirus OC43, (ix) the polymerase gene of each of the coronaviruses NL, 229E and SARS-CoV, as well as (x) the hexon region of adenoviruses serotypes associated with VRTIs.

The oligonucleotide sequence of primers or probes may be derived from either strand of the duplex DNA. In some instances, the primers or probes may include any of the bases A, G, C, or T or analogs and they may be degenerated at one or more chosen nucleotide position(s) to ensure the ubiquity of amplification from all strains of a target viral species.

Degenerated primers are a set of primers which have a number of options at mismatch positions in the sequence to allow annealing to an amplification of a variety of related sequences. For example, the following primer AYATTAGTGCTTTTAAAGCC may be in an equimolar mix of the primers ACATTAGTGCTTTTAAAGCC and ATATTAGTGCTTTTAAAGCC. Degeneracies obviously reduce the specificity of the primer(s), meaning mismatch opportunities are greater, and background noise increases; also, increased degeneracy means concentration of the individual primers decreases; hence, greater than 512-fold degeneracy is preferably avoided. Thus, degenerated primers should be carefully designed in order to avoid affecting the sensitivity and/or specificity of the assay. Inosine is a modified base that can bind with any of the regular base (A, T, C or G). Inosine is used in order to minimize the number of degeneracies in an oligonucleotide.

Codes of the International Union of Biochemistry (IUB)

### IUB Coding for A: Adenine C: Cytosine G: Guanine T: Thymine U: Uridine I: Inosine

| Bases | Code |
|---|---|
| A or C | M |
| A or G | R |
| A or T | W |
| C or G | S |
| C or T | Y |
| G or T | K |
| Inosine | I |

Several primers have been designed to efficiently amplify the pathogens described herein. It is to be understood that each of the oligonucleotides individually possess their own utility as it may be possible to use such oligonucleotides for other purposes than those described herein. For example, primers of the present invention may be combined with other primers for amplification of a longer or shorther amplicon. Probes of the present invention may be combined with other probes in detection tools such as microarrays.

The present invention features primers capable of specific amplification of a desired respiratory virus species.

Aspects of the invention relate to individual primers, primer pairs or combination of primers or primer pairs for used in the methods and kits of the present invention. Exemplary embodiments of primers, primer pairs and primer combinations are found below.

The present invention relates in one aspect thereof to an oligonucleotide of from 10 to 50 nucleotides long capable of specific binding to a gene selected from the group consisting of (i) a matrix gene of influenza A viruses (ii) a matrix gene of influenza B viruses, (iii) a nucleocapsid gene of human respiratory syncytial viruses, (iv) a nucleocapsid gene of human metapneumoviruses, (v) a 5′-non-coding region of human enteroviruses, (vi) a 5′-non-coding region of rhinoviruses, (vii) a fusion gene of parainfluenza viruses type 1, (viii) a fusion gene of parainfluenza viruses type 2, (ix) a fusion gene of parainfluenza viruses type 3, (x) a fusion gene of parainfluenza viruses type 4, (xi) a matrix gene of the coronaviruses OC43, (xii) a polymerase gene of coronaviruses NL, (xiii) a polymerase gene of coronaviruses 229E, (xiv) a polymerase gene of coronaviruses SARS-CoV and (xv) a hexon region of adenoviruses of serotypes associated with respiratory infections.

In accordance with the present invention, the oligonucleotide preferably binds to the gene of one respiratory virus species and not the gene of the other respiratory virus species. Also in accordance with the present invention, the oligonucleotide is capable of specific binding to the gene of one respiratory virus species and not the gene of the other respiratory virus species.

Exemplary embodiments of individual primers include the following.

The present invention provides in a first embodiment, a nucleic acid which may comprise from 0 to 5 nucleotide addition or deletion at a 5′ end of SEQ ID NO.:78.

In another embodiment, the present invention provides nucleic acid which may comprise from 0 to 5 nucleotide addition or deletion at a 5′ end of SEQ ID NO.:79.

In a further embodiment, the present invention provides a nucleic acid which may comprise from 0 to 5 nucleotide addition or deletion at a 5′ end of SEQ ID NO.:80.

In yet a further embodiment, the present invention provides a nucleic acid which may comprise from 0 to 5 nucleotide addition or deletion at a 5′ end of SEQ ID NO.:81.

In an additional embodiment, the present invention provides a nucleic acid which may comprise from 0 to 5 nucleotides addition or deletion at a 5′ end of SEQ ID NO.:82.

In yet an additional embodiment, the present invention provides a nucleic acid which may comprise from 0 to 5 nucleotide addition or deletion at a 5′ end of SEQ ID NO.:83.

In another exemplary embodiment, the present invention provides a nucleic acid which may comprise from 0 to 5 nucleotide addition or deletion at a 5′ end of SEQ ID NO.: 84.

In yet another exemplary embodiment, the present invention provides and a nucleic acid which may comprise from 0 to 5 nucleotide addition or deletion at a 5′ end of SEQ ID NO.:85.

In still another embodiment, the present invention provides a nucleic acid which may comprise from 0 to 5 nucleotide addition or deletion at a 5′ end of SEQ ID NO.:86.

In an additional embodiment, the present invention provides a nucleic acid which may comprise from 0 to 5 nucleotide addition or deletion at a 5′ end of SEQ ID NO.:87.

In still another embodiment, the present invention provides a nucleic acid which may comprise from 0 to 5 nucleotide addition or deletion at a 5′ end of SEQ ID NO.:88.

An additional embodiment of the present invention relates to a nucleic acid which may comprise from 0 to 5 nucleotide addition or deletion at a 5′ end of SEQ ID NO.:89.

Yet an additional exemplary embodiment of the present invention provides a nucleic acid which may comprise from 0 to 5 nucleotide addition or deletion at a 5′ end of SEQ ID NO.:90.

A further embodiment of the invention relates to a nucleic acid which may comprise from 0 to 5 nucleotide addition or deletion at a 5′ end of SEQ ID NO.:91.

Another embodiment of the invention relates to a nucleic acid which may comprise from 0 to 5 nucleotide addition or deletion at a 5′ end of SEQ ID NO.:92.

Yet another embodiment of the invention relates to a nucleic acid which may comprise from 0 to 5 nucleotide addition or deletion at a 5′ end of SEQ ID NO.:93.

An additional embodiment of the invention relates to a nucleic acid which may comprise from 0 to 5 nucleotide addition or deletion at a 5′ end of SEQ ID NO.:94.

Still an additional embodiment of the invention relates to a nucleic acid which may comprise from 0 to 5 nucleotide addition or deletion at a 5′ end of SEQ ID NO.:95.

In a further exemplary embodiment, the present invention provides a nucleic acid which may comprise from 0 to 5 nucleotide addition or deletion at a 5′ end of SEQ ID NO.:96.

In yet a further exemplary embodiment, the present invention provides a nucleic acid which may comprise from 0 to 5 nucleotide addition or deletion at a 5′ end of SEQ ID NO.:97.

In an additional exemplary embodiment, the present invention provides a nucleic acid which may comprise from 0 to 5 nucleotide addition or deletion at a 5′ end of SEQ ID NO.:98.

In yet an additional exemplary embodiment, the present invention provides a nucleic acid which may comprise from 0 to 5 nucleotide addition or deletion at a 5′ end of SEQ ID NO.:99.

Another embodiment of the invention relates to a nucleic acid which may comprise from 0 to 5 nucleotide addition or deletion at a 5′ end of SEQ ID NO.:100.

Still other embodiment of the invention relates to a nucleic acid which may comprise from 0 to 5 nucleotide addition or deletion at a 5′ end of SEQ ID NO.:101.

A further embodiment of the invention relates to a nucleic acid which may comprise from 0 to 5 nucleotide addition or deletion at a 5′ end of SEQ ID NO.:102.

Still a further embodiment of the invention relates to a nucleic acid which may comprise from 0 to 5 nucleotide addition or deletion at a 5′ end of SEQ ID NO.:103.

The invention also relates to a mixture or a combinations of oligonucleotides which may comprise at least two of the nucleic acids described above.

For the purpose of carrying out the methods of the invention, several sets of primers have been selected. Each set of primers may comprise at least one primer capable of specific amplification of the genetic material. The tested sample may thus be exposed with the sets of primers under conditions suitable for nucleic acid amplification. In accordance with an embodiment of the invention, the sets of primers may be capable of amplifying the genetic material of the virus under similar amplification conditions.

Exemplary embodiments of primer pairs include the following.

A primer pair comprising a nucleic acid which may comprise from 0 to 5 nucleotide addition or deletion at a 5′ end of SEQ ID NO.:78 and a nucleic acid which may comprise from 0 to 5 nucleotide addition or deletion at a 5′ end of SEQ ID NO.:79.

In another embodiment, the present invention relates to a primer pair comprising a nucleic acid which may comprise from 0 to 5 nucleotide addition or deletion at a 5′ end of SEQ ID NO.:80 and a nucleic acid which may comprise from 0 to 5 nucleotide addition or deletion at a 5′ end of SEQ ID NO.:81.

In an additional embodiment, the present invention relates to a primer pair comprising a nucleic acid which may comprise from 0 to 5 nucleotides addition or deletion at a 5′ end of SEQ ID NO.:82 and a nucleic acid which may comprise from 0 to 5 nucleotide addition or deletion at a 5′ end of SEQ ID NO.:83.

In accordance with the present invention, the human enterovirus, the rhinovirus, the human respiratory syncytial virus and the human metapneumovirus may be amplified by the combination or mixture of primer pairs defined above.

In yet an additional exemplary embodiment, the present invention relates to a primer pair comprising a nucleic acid which may comprise from 0 to 5 nucleotide addition or deletion at a 5′ end of SEQ ID NO.: 84 and a nucleic acid which may comprise from 0 to 5 nucleotide addition or deletion at a 5′ end of SEQ ID NO.:85.

In another embodiment, the present invention relates to a primer pair comprising a nucleic acid which may comprise from 0 to 5 nucleotide addition or deletion at a 5′ end of SEQ ID NO.:86 and a nucleic acid which may comprise from 0 to 5 nucleotide addition or deletion at a 5′ end of SEQ ID NO.:87.

In still another exemplary embodiment, the present invention relates to a primer pair comprising a nucleic acid which may comprise from 0 to 5 nucleotide addition or deletion at a 5′ end of SEQ ID NO.:88 and a nucleic acid which may comprise from 0 to 5 nucleotide addition or deletion at a 5′ end of SEQ ID NO.:89.

In a further exemplary embodiment, the present invention relates to a primer pair comprising a nucleic acid which may comprise from 0 to 5 nucleotide addition or deletion at a 5′ end of SEQ ID NO.:90 and a nucleic acid which may comprise from 0 to 5 nucleotide addition or deletion at a 5′ end of SEQ ID NO.:91.

In accordance with the present invention, the influenza A, the parainfluenza type 1, parainfluenza type 2 and the parainfluenza type 3 may be amplified by the combination or mixture of primer pairs defined above.

In yet a further embodiment, the present invention relates to a primer pair comprising a nucleic acid which may comprise from 0 to 5 nucleotide addition or deletion at a 5′ end of SEQ ID NO.:92 and a nucleic acid which may comprise from 0 to 5 nucleotide addition or deletion at a 5′ end of SEQ ID NO.:93.

In another exemplary embodiment, the present invention relates to a primer pair comprising a nucleic acid which may comprise from 0 to 5 nucleotide addition or deletion at a 5′ end of SEQ ID NO.:94 and a nucleic acid which may comprise from 0 to 5 nucleotide addition or deletion at a 5′ end of SEQ ID NO.:95.

In still another embodiment, the present invention relates to a primer pair comprising a nucleic acid which may comprise from 0 to 5 nucleotide addition or deletion at a 5′ end of SEQ ID NO.:96 and a nucleic acid which may comprise from 0 to 5 nucleotide addition or deletion at a 5′ end of SEQ ID NO.:97.

In accordance with the present invention, the coronaviruses SARS-CoV, 229E, NL and OC43 may be amplified by the combination or mixture of primer pairs defined above.

Another exemplary embodiment the present invention relates to a primer pair comprising a nucleic acid which may comprise from 0 to 5 nucleotide addition or deletion at a 5′ end of SEQ ID NO.:98 and a nucleic acid which may comprise from 0 to 5 nucleotide addition or deletion at a 5′ end of SEQ ID NO.:99.

In an additional exemplary embodiment, the present invention relates to a primer pair comprising a nucleic acid which may comprise from 0 to 5 nucleotide addition or deletion at a 5′ end of SEQ ID NO.:100 and a nucleic acid which may comprise from 0 to 5 nucleotide addition or deletion at a 5′ end of SEQ ID NO.:101.

In a further exemplary embodiment, the present invention relates to a primer pair comprising a nucleic acid which may comprise from 0 to 5 nucleotide addition or deletion at a 5′ end of SEQ ID NO.:102 and a nucleic acid which may comprise from 0 to 5 nucleotide addition or deletion at a 5′ end of SEQ ID NO.:103.

In accordance with the present invention, the adenovirus, the influenza B and the parainfluenza type 4 may be amplified by the combination or mixture of primer pairs defined above.

More specific embodiments of selected primer pairs and probes are listed in Table 2. These primer pairs have more particularly been selected for their specificity, sensitivity, and their capacity to amplify all or most members within each target species or genus. Public database analysis indeed indicates that the nucleic acid sequences amplified with the primer pairs described in Table 2 are specific for only one of the 15 target viruses.

The present invention also features hybridization probes chosen from the regions amplified with the PCR primer pairs described above, i.e., binding within the PCR amplicon.

Other aspects of the invention relates to oligonucleotides which may comprise individual probes and probe combinations which may be used in the methods and kits described herein. In order to carry out the invention, tested sample may be exposed with the probe under conditions suitable for hybridization. In accordance with an embodiment of the invention, the probe may be capable of hybridizing to the genetic material under similar hybridization conditions.

A nucleic acid which may comprise from 0 to 5 nucleotide addition, deletion or combination of addition and deletion at a 5′ end and/or 3′ end thereof of any one of the probes listed in Table 2 or a complement thereof is encompassed by the present invention. It is to be understood herein that the language recited is to be applied for each nucleic acid sequences individually or collectively.

Exemplary embodiments of probes includes the following:

A nucleic acid which may comprise from 0 to 5 nucleotide addition, deletion or combination of addition and deletion at a 5′ end and/or 3′ end thereof of SEQ ID NO.:106 or a complement thereof.

A nucleic acid which may comprise from 0 to 5 nucleotide addition, deletion or combination of addition and deletion at a 5′ end and/or 3′ end thereof of SEQ ID NO.:107 or a complement thereof.

A nucleic acid which may comprise from 0 to 5 nucleotide addition, deletion or combination of addition and deletion at a 5′ end and/or 3′ end thereof of SEQ ID NO.:108 or a complement thereof.

A nucleic acid which may comprise from 0 to 5 nucleotide addition, deletion or combination of addition and deletion at a 5′ end and/or 3′ end thereof of SEQ ID NO.:109 or a complement thereof.

A nucleic acid which may comprise from 0 to 5 nucleotide addition, deletion or combination of addition and deletion at a 5′ end and/or 3′ end thereof of SEQ ID NO.:110 or a complement thereof.

A nucleic acid which may comprise from 0 to 5 nucleotide addition, deletion or combination of addition and deletion at a 5′ end and/or 3′ end thereof of SEQ ID NO.:111 or a complement thereof.

A nucleic acid which may comprise from 0 to 5 nucleotide addition, deletion or combination of addition and deletion at a 5′ end and/or 3′ end thereof of SEQ ID NO.:112 or a complement thereof.

A nucleic acid which may comprise from 0 to 5 nucleotide addition, deletion or combination of addition and deletion at a 5′ end and/or 3′ end thereof of SEQ ID NO.:113 or a complement thereof.

A nucleic acid which may comprise from 0 to 5 nucleotide addition, deletion or combination of addition and deletion at a 5′ end and/or 3′ end thereof of SEQ ID NO.:114 or a complement thereof.

A nucleic acid which may comprise from 0 to 5 nucleotide addition, deletion or combination of addition and deletion at a 5′ end and/or 3′ end thereof of SEQ ID NO.:115 or a complement thereof.

A nucleic acid which may comprise from 0 to 5 nucleotide addition, deletion or combination of addition and deletion at a 5′ end and/or 3′ end thereof of SEQ ID NO.:116 or a complement thereof.

A nucleic acid which may comprise from 0 to 5 nucleotide addition, deletion or combination of addition and deletion at a 5′ end and/or 3′ end thereof of SEQ ID NO.:117 or a complement thereof.

A nucleic acid which may comprise from 0 to 5 nucleotide addition, deletion or combination of addition and deletion at a 5′ end and/or 3′ end thereof of SEQ ID NO.:118 or a complement thereof.

A nucleic acid which may comprise from 0 to 5 nucleotide addition, deletion or combination of addition and deletion at a 5′ end and/or 3′ end thereof of SEQ ID NO.:119 or a complement thereof.

A nucleic acid which may comprise from 0 to 5 nucleotide addition, deletion or combination of addition and deletion at a 5′ end and/or 3′ end thereof of SEQ ID NO.:120 or a complement thereof.

A nucleic acid which may comprise from 0 to 5 nucleotide addition, deletion or combination of addition and deletion at a 5′ end and/or 3′ end thereof of SEQ ID NO.:194 or a complement thereof.

A nucleic acid which may comprise from 0 to 5 nucleotide addition, deletion or combination of addition and deletion at a 5′ end and/or 3′ end thereof of SEQ ID NO.:195 or a complement thereof.

A nucleic acid which may comprise from 0 to 5 nucleotide addition, deletion or combination of addition and deletion at a 5′ end and/or 3′ end thereof of SEQ ID NO.:196 or a complement thereof.

A nucleic acid which may comprise from 0 to 5 nucleotide addition, deletion or combination of addition and deletion at a 5′ end and/or 3′ end thereof of SEQ ID NO.:121 or a complement thereof.

A nucleic acid which may comprise from 0 to 5 nucleotide addition, deletion or combination of addition and deletion at a 5′ end and/or 3′ end thereof of SEQ ID NO.:122 or a complement thereof.

A nucleic acid which may comprise from 0 to 5 nucleotide addition, deletion or combination of addition and deletion at a 5′ end and/or 3′ end thereof of SEQ ID NO.:123 or a complement thereof.

A nucleic acid which may comprise from 0 to 5 nucleotide addition, deletion or combination of addition and deletion at a 5′ end and/or 3′ end thereof of SEQ ID NO.:124 or a complement thereof.

A nucleic acid which may comprise from 0 to 5 nucleotide addition, deletion or combination of addition and deletion at a 5′ end and/or 3′ end thereof of SEQ ID NO.:125 or a complement thereof.

A nucleic acid which may comprise from 0 to 5 nucleotide addition, deletion or combination of addition and deletion at a 5′ end and/or 3′ end thereof of SEQ ID NO.:126 or a complement thereof.

A nucleic acid which may comprise from 0 to 5 nucleotide addition, deletion or combination of addition and deletion at a 5′ end and/or 3′ end thereof of SEQ ID NO.:127 or a complement thereof.

A nucleic acid which may comprise from 0 to 5 nucleotide addition, deletion or combination of addition and deletion at a 5′ end and/or 3′ end thereof of SEQ ID NO.:197 or a complement thereof.

A nucleic acid which may comprise from 0 to 5 nucleotide addition, deletion or combination of addition and deletion at a 5′ end and/or 3′ end thereof of SEQ ID NO.:198 or a complement thereof.

A nucleic acid which may comprise from 0 to 5 nucleotide addition, deletion or combination of addition and deletion at a 5′ end and/or 3′ end thereof of SEQ ID NO.:199 or a complement thereof.

A nucleic acid which may comprise from 0 to 5 nucleotide addition, deletion or combination of addition and deletion at a 5′ end and/or 3′ end thereof of SEQ ID NO.:200 or a complement thereof.

A nucleic acid which may comprise from 0 to 5 nucleotide addition, deletion or combination of addition and deletion at a 5′ end and/or 3′ end thereof of SEQ ID NO.:128 or a complement thereof.

A nucleic acid which may comprise from 0 to 5 nucleotide addition, deletion or combination of addition and deletion at a 5′ end and/or 3′ end thereof of SEQ ID NO.:129 or a complement thereof.

A nucleic acid which may comprise from 0 to 5 nucleotide addition, deletion or combination of addition and deletion at a 5′ end and/or 3′ end thereof of SEQ ID NO.:130 or a complement thereof.

A nucleic acid which may comprise from 0 to 5 nucleotide addition, deletion or combination of addition and deletion at a 5′ end and/or 3′ end thereof of SEQ ID NO.:131 or a complement thereof.

A nucleic acid which may comprise from 0 to 5 nucleotide addition, deletion or combination of addition and deletion at a 5′ end and/or 3′ end thereof of SEQ ID NO.:132 or a complement thereof.

A nucleic acid which may comprise from 0 to 5 nucleotide addition, deletion or combination of addition and deletion at a 5′ end and/or 3′ end thereof of SEQ ID NO.:133 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 1.

Other exemplary embodiments of individual probes include the following:

A nucleic acid which may comprise from 0 to 5 nucleotide addition, deletion or combination of addition and deletion at a 5′ end and/or 3′ end thereof of SEQ ID NO.:134 or a complement thereof.

A nucleic acid which may comprise from 0 to 5 nucleotide addition, deletion or combination of addition and deletion at a 5′ end and/or 3′ end thereof of SEQ ID NO.:135 or a complement thereof.

A nucleic acid which may comprise from 0 to 5 nucleotide addition, deletion or combination of addition and deletion at a 5′ end and/or 3′ end thereof of SEQ ID NO.:136 or a complement thereof.

A nucleic acid which may comprise from 0 to 5 nucleotide addition, deletion or combination of addition and deletion at a 5′ end and/or 3′ end thereof of SEQ ID NO.:137 or a complement thereof.

A nucleic acid which may comprise from 0 to 5 nucleotide addition, deletion or combination of addition and deletion at a 5′ end and/or 3′ end thereof of SEQ ID NO.:138 or a complement thereof.

A nucleic acid which may comprise from 0 to 5 nucleotide addition, deletion or combination of addition and deletion at a 5′ end and/or 3′ end thereof of SEQ ID NO.:139 or a complement thereof.

A nucleic acid which may comprise from 0 to 5 nucleotide addition, deletion or combination of addition and deletion at a 5′ end and/or 3′ end thereof of SEQ ID NO.:1 40 or a complement thereof.

A nucleic acid which may comprise from 0 to 5 nucleotide addition, deletion or combination of addition and deletion at a 5′ end and/or 3′ end thereof of SEQ ID NO.:141 or a complement thereof.

A nucleic acid which may comprise from 0 to 5 nucleotide addition, deletion or combination of addition and deletion at a 5′ end and/or 3′ end thereof of SEQ ID NO.:142 or a complement thereof.

A nucleic acid which may comprise from 0 to 5 nucleotide addition, deletion or combination of addition and deletion at a 5′ end and/or 3′ end thereof of SEQ ID NO.:143 or a complement thereof.

A nucleic acid which may comprise from 0 to 5 nucleotide addition, deletion or combination of addition and deletion at a 5′ end and/or 3′ end thereof of SEQ ID NO.:144 or a complement thereof.

A nucleic acid which may comprise from 0 to 5 nucleotide addition, deletion or combination of addition and deletion at a 5′ end and/or 3′ end thereof of SEQ ID NO.:145 or a complement thereof.

A nucleic acid which may comprise from 0 to 5 nucleotide addition, deletion or combination of addition and deletion at a 5′ end and/or 3′ end thereof of SEQ ID NO.:146 or a complement thereof.

A nucleic acid which may comprise from 0 to 5 nucleotide addition, deletion or combination of addition and deletion at a 5′ end and/or 3′ end thereof of SEQ ID NO.:147 or a complement thereof.

A nucleic acid which may comprise from 0 to 5 nucleotide addition, deletion or combination of addition and deletion at a 5′ end and/or 3′ end thereof of SEQ ID NO.:148 or a complement thereof.

A nucleic acid which may comprise from 0 to 5 nucleotide addition, deletion or combination of addition and deletion at a 5′ end and/or 3′ end thereof of SEQ ID NO.:149 or a complement thereof.

A nucleic acid which may comprise from 0 to 5 nucleotide addition, deletion or combination of addition and deletion at a 5′ end and/or 3′ end thereof of SEQ ID NO.:150 or a complement thereof.

A nucleic acid which may comprise from 0 to 5 nucleotide addition, deletion or combination of addition and deletion at a 5′ end and/or 3′ end thereof of SEQ ID NO.:151 or a complement thereof.

A nucleic acid which may comprise from 0 to 5 nucleotide addition, deletion or combination of addition and deletion at a 5′ end and/or 3′ end thereof of SEQ ID NO.:152 or a complement thereof.

A nucleic acid which may comprise from 0 to 5 nucleotide addition, deletion or combination of addition and deletion at a 5′ end and/or 3′ end thereof of SEQ ID NO.:201 or a complement thereof.

A nucleic acid which may comprise from 0 to 5 nucleotide addition, deletion or combination of addition and deletion at a 5′ end and/or 3′ end thereof of SEQ ID NO.:202 or a complement thereof.

A nucleic acid which may comprise from 0 to 5 nucleotide addition, deletion or combination of addition and deletion at a 5′ end and/or 3′ end thereof of SEQ ID NO.:203 or a complement thereof.

A nucleic acid which may comprise from 0 to 5 nucleotide addition, deletion or combination of addition and deletion at a 5′ end and/or 3′ end thereof of SEQ ID NO.:204 or a complement thereof.

A nucleic acid which may comprise from 0 to 5 nucleotide addition, deletion or combination of addition and deletion at a 5′ end and/or 3′ end thereof of SEQ ID NO.:205 or a complement thereof.

A nucleic acid which may comprise from 0 to 5 nucleotide addition, deletion or combination of addition and deletion at a 5′ end and/or 3′ end thereof of SEQ ID NO.:206 or a complement thereof.

A nucleic acid which may comprise from 0 to 5 nucleotide addition, deletion or combination of addition and deletion at a 5′ end and/or 3′ end thereof of SEQ ID NO.:207 or a complement thereof.

A nucleic acid which may comprise from 0 to 5 nucleotide addition, deletion or combination of addition and deletion at a 5′ end and/or 3′ end thereof of SEQ ID NO.:208 or a complement thereof.

A nucleic acid which may comprise from 0 to 5 nucleotide addition, deletion or combination of addition and deletion at a 5′ end and/or 3′ end thereof of SEQ ID NO.:153 or a complement thereof.

A nucleic acid which may comprise from 0 to 5 nucleotide addition, deletion or combination of addition and deletion at a 5′ end and/or 3′ end thereof of SEQ ID NO.:154 or a complement thereof.

A nucleic acid which may comprise from 0 to 5 nucleotide addition, deletion or combination of addition and deletion at a 5′ end and/or 3′ end thereof of SEQ ID NO.:155 or a complement thereof.

A nucleic acid which may comprise from 0 to 5 nucleotide addition, deletion or combination of addition and deletion at a 5′ end and/or 3′ end thereof of SEQ ID NO.:156 or a complement thereof.

A nucleic acid which may comprise from 0 to 5 nucleotide addition, deletion or combination of addition and deletion at a 5′ end and/or 3′ end thereof of SEQ ID NO.:157 or a complement thereof.

A nucleic acid which may comprise from 0 to 5 nucleotide addition, deletion or combination of addition and deletion at a 5′ end and/or 3′ end thereof of SEQ ID NO.:158 or a complement thereof.

A nucleic acid which may comprise from 0 to 5 nucleotide addition, deletion or combination of addition and deletion at a 5′ end and/or 3′ end thereof of SEQ ID NO.:209 or a complement thereof.

A nucleic acid which may comprise from 0 to 5 nucleotide addition, deletion or combination of addition and deletion at a 5′ end and/or 3′ end thereof of SEQ ID NO.:225 or a complement thereof.

A nucleic acid which may comprise from 0 to 5 nucleotide addition, deletion or combination of addition and deletion at a 5′ end and/or 3′ end thereof of SEQ ID NO.:226 or a complement thereof.

A nucleic acid which may comprise from 0 to 5 nucleotide addition, deletion or combination of addition and deletion at a 5′ end and/or 3′ end thereof of SEQ ID NO.:227 or a complement thereof.

A nucleic acid which may comprise from 0 to 5 nucleotide addition, deletion or combination of addition and deletion at a 5′ end and/or 3′ end thereof of SEQ ID NO.:228 or a complement thereof.

A nucleic acid which may comprise from 0 to 5 nucleotide addition, deletion or combination of addition and deletion at a 5′ end and/or 3′ end thereof of SEQ ID NO.:229 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 2.

Yet other exemplary embodiments of individual probes include those of Table 2 identified for Multiplex 3 such as the following:

A nucleic acid which may comprise from 0 to 5 nucleotide addition, deletion or combination of addition and deletion at a 5′ end and/or 3′ end thereof of SEQ ID NO.:159 or a complement thereof.

A nucleic acid which may comprise from 0 to 5 nucleotide addition, deletion or combination of addition and deletion at a 5′ end and/or 3′ end thereof of SEQ ID NO.:160 or a complement thereof.

A nucleic acid which may comprise from 0 to 5 nucleotide addition, deletion or combination of addition and deletion at a 5′ end and/or 3′ end thereof of SEQ ID NO.:161 or a complement thereof.

A nucleic acid which may comprise from 0 to 5 nucleotide addition, deletion or combination of addition and deletion at a 5′ end and/or 3′ end thereof of SEQ ID NO.:162 or a complement thereof.

A nucleic acid which may comprise from 0 to 5 nucleotide addition, deletion or combination of addition and deletion at a 5′ end and/or 3′ end thereof of SEQ ID NO.:163 or a complement thereof.

A nucleic acid which may comprise from 0 to 5 nucleotide addition, deletion or combination of addition and deletion at a 5′ end and/or 3′ end thereof of SEQ ID NO.:164 or a complement thereof.

Further exemplary embodiments of individual probes include the following:

A nucleic acid which may comprise from 0 to 5 nucleotide addition, deletion or combination of addition and deletion at a 5′ end and/or 3′ end thereof of SEQ ID NO.:165 or a complement thereof.

A nucleic acid which may comprise from 0 to 5 nucleotide addition, deletion or combination of addition and deletion at a 5′ end and/or 3′ end thereof of SEQ ID NO.:166 or a complement thereof.

A nucleic acid which may comprise from 0 to 5 nucleotide addition, deletion or combination of addition and deletion at a 5′ end and/or 3′ end thereof of SEQ ID NO.:167 or a complement thereof.

A nucleic acid which may comprise from 0 to 5 nucleotide addition, deletion or combination of addition and deletion at a 5′ end and/or 3′ end thereof of SEQ ID NO.:168 or a complement thereof.

A nucleic acid which may comprise from 0 to 5 nucleotide addition, deletion or combination of addition and deletion at a 5′ end and/or 3′ end thereof of SEQ ID NO.:169 or a complement thereof.

A nucleic acid which may comprise from 0 to 5 nucleotide addition, deletion or combination of addition and deletion at a 5′ end and/or 3′ end thereof of SEQ ID NO.:170 or a complement thereof.

A nucleic acid which may comprise from 0 to 5 nucleotide addition, deletion or combination of addition and deletion at a 5′ end and/or 3′ end thereof of SEQ ID NO.:171 or a complement thereof.

A nucleic acid which may comprise from 0 to 5 nucleotide addition, deletion or combination of addition and deletion at a 5′ end and/or 3′ end thereof of SEQ ID NO.:172 or a complement thereof.

A nucleic acid which may comprise from 0 to 5 nucleotide addition, deletion or combination of addition and deletion at a 5′ end and/or 3′ end thereof of SEQ ID NO.:173 or a complement thereof.

A nucleic acid which may comprise from 0 to 5 nucleotide addition, deletion or combination of addition and deletion at a 5′ end and/or 3′ end thereof of SEQ ID NO.:174 or a complement thereof.

A nucleic acid which may comprise from 0 to 5 nucleotide addition, deletion or combination of addition and deletion at a 5′ end and/or 3′ end thereof of SEQ ID NO.:175 or a complement thereof.

A nucleic acid which may comprise from 0 to 5 nucleotide addition, deletion or combination of addition and deletion at a 5′ end and/or 3′ end thereof of SEQ ID NO.:176 or a complement thereof.

A nucleic acid which may comprise from 0 to 5 nucleotide addition, deletion or combination of addition and deletion at a 5′ end and/or 3′ end thereof of SEQ ID NO.:177 or a complement thereof.

A nucleic acid which may comprise from 0 to 5 nucleotide addition, deletion or combination of addition and deletion at a 5′ end and/or 3′ end thereof of SEQ ID NO.:178 or a complement thereof.

A nucleic acid which may comprise from 0 to 5 nucleotide addition, deletion or combination of addition and deletion at a 5′ end and/or 3′ end thereof of SEQ ID NO.:179 or a complement thereof.

A nucleic acid which may comprise from 0 to 5 nucleotide addition, deletion or combination of addition and deletion at a 5′ end and/or 3′ end thereof of SEQ ID NO.:180 or a complement thereof.

A nucleic acid which may comprise from 0 to 5 nucleotide addition, deletion or combination of addition and deletion at a 5′ end and/or 3′ end thereof of SEQ ID NO.:181 or a complement thereof.

A nucleic acid which may comprise from 0 to 5 nucleotide addition, deletion or combination of addition and deletion at a 5′ end and/or 3′ end thereof of SEQ ID NO.:182 or a complement thereof.

A nucleic acid which may comprise from 0 to 5 nucleotide addition, deletion or combination of addition and deletion at a 5′ end and/or 3′ end thereof of SEQ ID NO.:183 or a complement thereof.

A nucleic acid which may comprise from 0 to 5 nucleotide addition, deletion or combination of addition and deletion at a 5′ end and/or 3′ end thereof of SEQ ID NO.:210 or a complement thereof.

A nucleic acid which may comprise from 0 to 5 nucleotide addition, deletion or combination of addition and deletion at a 5′ end and/or 3′ end thereof of SEQ ID NO.:211 or a complement thereof.

A nucleic acid which may comprise from 0 to 5 nucleotide addition, deletion or combination of addition and deletion at a 5′ end and/or 3′ end thereof of SEQ ID NO.:212 or a complement thereof.

A nucleic acid which may comprise from 0 to 5 nucleotide addition, deletion or combination of addition and deletion at a 5′ end and/or 3′ end thereof of SEQ ID NO.:213 or a complement thereof.

A nucleic acid which may comprise from 0 to 5 nucleotide addition, deletion or combination of addition and deletion at a 5′ end and/or 3′ end thereof of SEQ ID NO.:214 or a complement thereof.

A nucleic acid which may comprise from 0 to 5 nucleotide addition, deletion or combination of addition and deletion at a 5′ end and/or 3′ end thereof of SEQ ID NO.:184 or a complement thereof.

A nucleic acid which may comprise from 0 to 5 nucleotide addition, deletion or combination of addition and deletion at a 5′ end and/or 3′ end thereof of SEQ ID NO.:185 or a complement thereof.

A nucleic acid which may comprise from 0 to 5 nucleotide addition, deletion or combination of addition and deletion at a 5′ end and/or 3′ end thereof of SEQ ID NO.:186 or a complement thereof.

A nucleic acid which may comprise from 0 to 5 nucleotide addition, deletion or combination of addition and deletion at a 5′ end and/or 3′ end thereof of SEQ ID NO.:187 or a complement thereof.

A nucleic acid which may comprise from 0 to 5 nucleotide addition, deletion or combination of addition and deletion at a 5′ end and/or 3′ end thereof of SEQ ID NO.:188 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 4.

The oligonucleotide (i.e., nucleic acid, e.g., primers and/or probes) may comprise a label. The label may be found within the nucleic acid. The label may be attached to a 5′-end of the nucleic acid. The label may be attached to a 3′-end of the oligonucleotide.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:106, SEQ ID NO.:107, SEQ ID NO.:108, SEQ ID NO.:109, SEQ ID NO.:110, SEQ ID NO.:111, SEQ ID NO.:112, SEQ ID NO.:113, SEQ ID NO.:114, SEQ ID NO.:115, SEQ ID NO.:116, SEQ ID NO.:117, SEQ ID NO.:118, SEQ ID NO.:119, SEQ ID NO.:120, SEQ ID NO.:194, SEQ ID NO.:195 and SEQ ID NO.:196 or complement thereof may be used individually or collectively (including combinations of all probes and sub-combinations of at least two probes) for the detection of a rhinovirus and/or an enterovirus.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:121, SEQ ID NO.:122, SEQ ID NO.:123, SEQ ID NO.:124, SEQ ID NO.:125, SEQ ID NO.:126, SEQ ID NO.:127, SEQ ID NO.:197, SEQ ID NO.:198, SEQ ID NO.:199 and SEQ ID NO.:200 or complement thereof may be used individually or collectively (including combinations of all probes and sub-combinations of at least two probes) for the detection of a human respiratory syncytial virus.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:128, SEQ ID NO.:129, SEQ ID NO.:130, SEQ ID NO.:131, SEQ ID NO.:132 and SEQ ID NO.:133 or complement thereof may be used individually or collectively (including combinations of all probes and sub-combinations of at least two probes) for the detection of a human metapneumovirus.

In accordance with the present invention, each of the above oligonucleotides may be provided in a separate container or may be attached to a specific location on a solid support.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:134, SEQ ID NO.:135, SEQ ID NO.:136, SEQ ID NO.:137, SEQ ID NO.:138, SEQ ID NO.:139, SEQ ID NO.:140, SEQ ID NO.:141, SEQ ID NO.:142, SEQ ID NO.:143, SEQ ID NO.144, SEQ ID NO.:145, SEQ ID NO.:146, SEQ ID NO.:147, SEQ ID NO.:148, SEQ ID NO.:149, SEQ ID NO.:150, SEQ ID NO.:151, SEQ ID NO.:152, SEQ ID NO.:201, SEQ ID NO.:202, SEQ ID NO.:203, SEQ ID NO.:204, SEQ ID NO.:205, SEQ ID NO.:206, SEQ ID NO.:207 and SEQ ID NO.:208 or complement thereof may be used individually or collectively (including combinations of all probes and sub-combinations of at least two probes) for the detection of an influenza A virus.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:153 and SEQ ID NO.:154 or complement thereof may be used individually or collectively (including combinations of all probes and sub-combinations of at least two probes) for the detection of a parainfluenza type 1.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:155, SEQ ID NO.:156 and SEQ ID NO.:157 or complement thereof may be used individually or collectively (including combinations of all probes and sub-combinations of at least two probes) for the detection of a parainfluenza type 2.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:158, SEQ ID NO.:209 and SEQ ID NOs.:225 to 229 or complement thereof may be used individually or collectively (including combinations of all probes and sub-combinations of at least two probes) for the detection of a parainfluenza type 3.

In accordance with the present invention, each of the above oligonucleotides may be provided in a separate container or may be attached to a specific location on a solid support.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:159 and SEQ ID NO.:160 or complement thereof may be used individually or collectively (including combinations of all probes and sub-combinations of at least two probes) for the detection of a coronavirus SARS-CoV.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:161 or complement thereof may be used individually or collectively (including combinations of all probes and sub-combinations of at least two probes) for the detection of a coronavirus 229E.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:162 or complement thereof may be used individually or collectively (including combinations of all probes and sub-combinations of at least two probes) for the detection of a coronavirus NL.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:163 and SEQ ID NO.:164 or complement thereof may be used individually or collectively (including combinations of all probes and sub-combinations of at least two probes) for the detection of a coronavirus OC43.

In accordance with the present invention, each of the above oligonucleotides may be provided in a separate container or may be attached to a specific location on a solid support.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:165, SEQ ID NO.:166, SEQ ID NO.:167, SEQ ID NO.:168, SEQ ID NO.:169, SEQ ID NO.:170, SEQ ID NO.:171, SEQ ID NO.:172, SEQ ID NO.:173, SEQ ID NO.:174, SEQ ID NO.:175, SEQ ID NO.:176, SEQ ID NO.:177, SEQ ID NO.:178 or complement thereof may be used individually or collectively (including combinations of all probes and sub-combinations of at least two probes) for the detection of an adenovirus.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:179, SEQ ID NO.:180, SEQ ID NO.:181, SEQ ID NO.:182, SEQ ID NO.:183, SEQ ID NO.:210, SEQ ID NO.:211, SEQ ID NO.:212, SEQ ID NO.:213 and SEQ ID NO.:214 or complement thereof may be used individually or collectively (including combinations of all probes and sub-combinations of at least two probes) for the detection of an influenza B virus.

It is to be understood herein that any of the oligonucleotides having or consisting of the sequences selected from the group consisting of those comprising from 0 to 5 nucleotide addition, deletion or combination of addition and deletion at a 5′ end and/or 3′ end of SEQ ID NO.:184, SEQ ID NO.:185, SEQ ID NO.:186, SEQ ID NO.:187, SEQ ID NO.:188 or complement thereof may be used individually or collectively (including combinations of all probes and sub-combinations of at least two probes) for the detection of a parainfluenza 4 virus.

In accordance with the present invention, each of the above oligonucleotides may be provided in a separate container or may be attached to a specific location on a solid support.

More specific embodiments of probes selected for the optimal multiplex assays are listed in Table 2. These probes can be used for detecting the 15 targeted viruses by either hybridizing to target virus nucleic acids amplified with the selected primer pairs or to unamplified target virus nucleic acids using signal amplification methods such as ultra-sensitive biosensors. When a probe is combined with other probes for simultaneous detection of multiple viruses, the specificity of the probe should not be substantially affected by the presence of other probes, i.e., it still hybridizes to the target virus nucleic acid. Preferably, a probe selected for one virus does not hybridize to a nucleic acid from another virus.

The primers or probes may be of any suitable length determined by the user. In an embodiment of the present invention, the primers and/or probes (independently from one another) may be for example, from 10 to 50 nucleotide long (inclusively), from 10 to 40, from 10 to 35, from 10 to 30, from 12 to 40, from 12 to 25 nucleotide long (inclusively), from 15 to 25 nucleotide long (inclusively), from 15 to 20 nucleotides long (inclusively), etc. Although for purpose of concision, the complete list of combination of length between 10 to 50 nucleotides long is not provided herein it is intended that each and every possible combinations that may be found between 10 to 50 nucleotides (inclusively) be covered. A few example only of such possible combination is provided as follow, 12 to 25, 10 to 30, 11 to 30, 10 to 29, 11 to 29, 15 to 17, 14 to 21, etc.

As used herein the term “at least two” encompasses, “at least three”, “at least four”, “at least five”, “at least six”, “at least seven”, “at least eight”, “at least nine”, “at least ten”, “at least eleven”, “at least twelve”, “at least thirteen”, “at least fourteen”, “at least fifteen”, “at least sixteen”, “at least seventeen”, “at least eighteen”, “at least nineteen”, “at least twenty”, “at least twenty-one”, “at least twenty-two”, “at least twenty-three”, “at least twenty-four”, “at least twenty-five”, “at least twenty-six”, “at least twenty-seven”, “at least twenty-eight”, etc.

In another embodiment of the invention, the primers and/or probe (independently from one another) may be at least 10 nucleotides long, at least 11 nucleotides long, at least 12 nucleotides long, at least 13 nucleotides long, at least 14 nucleotides long, at least 15 nucleotides long, at least 16 nucleotides long, at least 17 nucleotides long, at least 18 nucleotides long, at least 19 nucleotides long, at least 20 nucleotides long, at least 21 nucleotides long, at least 22 nucleotides long, at least 23 nucleotides long, at least 24 nucleotides long, at least 25 nucleotides long, at least 26 nucleotides long, etc.

The primers and/or probes described in Table 2 may thus comprise additional nucleotides at their 5′ end and/or 3′ end. The identity of these nucleotides may vary. In some instances, the nucleotide may be chosen among the conventional A, T, G, or C bases while in other instances, the nucleotide may be a modified nucleotide as known in the art. However, in an embodiment of the invention, the additional nucleotide may correspond to the nucleotide found in any of the corresponding gene sequence listed in Table 1 or found in public databases.

As used herein the term “comprising from 0 to 5 additional nucleotides at a 5′ end and/or 3′ end thereof” means that the oligonucleotide or nucleic acid may have either, a) 0, 1, 2, 3, 4 or 5 additional nucleotide at its 5′ end, b) 0, 1, 2, 3, 4 or 5 additional nucleotide at its 3′ end or c) 0, 1, 2, 3, 4 or 5 additional nucleotide at its 5′ end and 0, 1, 2, 3, 4 or 5 additional nucleotide at its 3′ end.

As used herein the term “comprising from 0 to 5 nucleotides deletion at a 5′ end and/or 3′ end thereof” means that the oligonucleotide or nucleic acid may have either, a) 0, 1, 2, 3, 4 or 5 nucleotide deleted at its 5′ end, b) 0, 1, 2, 3, 4 or 5 nucleotide deleted at its 3′ end or c) 0, 1, 2, 3, 4 or 5 nucleotide deleted at its 5′ end and 0, 1, 2, 3, 4 or 5 nucleotide deleted at its 3′ end.

As used herein the term “comprising from 0 to 5 additional nucleotides at one of a 5′ end or 3′ end and/or a deletion of from 0 to 5 nucleotides at the other of a 5′ end or 3′ end” means that the oligonucleotide or nucleic acid may have either, a) 0, 1, 2, 3, 4 or 5 additional nucleotide at its 5′ end and 0, 1, 2, 3, 4 or 5 nucleotides deleted at its 3′ end or b) 0, 1, 2, 3, 4 or 5 additional nucleotide at its 3′ end and 0, 1, 2, 3, 4 or 5 nucleotides deleted at its 5′ end, c) 0, 1, 2, 3, 4 or 5 additional nucleotide at its 5′ end and 0, 1, 2, 3, 4 or 5 additional nucleotides at its 3′ end or d) 0, 1, 2, 3, 4 or 5 nucleotide deleted at its 5′ end and 0, 1, 2, 3, 4 or 5 nucleotides deleted at its 3′ end.

The term “comprising from 0 to 5” also encompasses “comprising from 1 to 5”, “comprising from 2 to 5”, “comprising from 3 to 5”; “comprising from 4 to 5”, “comprising from 0 to 4”, “comprising from 1 to 4”; “comprising from 2 to 4”, “comprising from 3 to 4”, “comprising from 0 to 3” “comprising from 1 to 3”; “comprising from 2 to 3”, “comprising from 0 to 2”, “comprising from 0 to 1”, “comprising 0”, “comprising 1”, “comprising 2”, “comprising 3”, “comprising 4”, or “comprising 5”.

In accordance with the present invention, the primers and/or probes may be labelled. In an embodiment of the invention, the primers may be labelled, therefore providing a labelled target amplicon. In accordance with the present invention the generated amplicons may detected by hybridization with genus- and/or species-specific capture probes. In other embodiments, it may be useful to label the probes.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³2P), phosphorescent labels, enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, each of which is hereby incorporated by reference in its entirety for all purposes. Fluorescent labels may easily be added during an in vitro transcription reaction and thus represent an interesting avenue.

In addition to the virus-specific oligonucleotides mentioned herein, the methods and kits may further comprise controls, such as control primers, control probes, control samples, etc. Although exemplary embodiments of controls have been provided in Table 4, a person of skill in the art will understand that any type of controls may be used to validate the methods.

### Methods of Amplification and Detection of Respiratory Viruses

The present invention provides in other aspects thereof, methods of detecting a respiratory virus which may comprise the step of: exposing a sample containing or suspected of containing a pathogen with oligonucleotide mixtures comprising multiple oligonucleotide species capable of specific binding with a genetic material of a respiratory virus species selected from the group consisting of (i) an influenza A virus (ii) an influenza B virus, (iii) a human respiratory syncytial virus (RSV), (iv) a human metapneumovirus (hMPV), (v) a human enterovirus, (vi) a rhinovirus, (vii) a parainfluenza virus type 1, (viii) a parainfluenza virus type 2, (ix) a parainfluenza virus type 3, (x) a parainfluenza virus type 4, (xi) a coronavirus OC43, (xii) a coronavirus NL, (xiii) a coronavirus 229E, (xiv) a coronavirus SARS-CoV and (xv) an adenovirus serotype associated with respiratory infections. In accordance with the present invention, the oligonucleotide mixtures may be capable of amplifying the genetic material under similar amplification conditions and/or may be capable of hybridizing to the genetic material under similar hybridization conditions.

The method is particularly applied to the detection of the target genes of the respiratory virus as described herein.

In accordance with the present invention, the multiple oligonucleotide species may comprise multiple sets of primer pairs which may be capable of specific amplification of the genetic material. The sample is thus exposed with the multiple sets of primer pairs under conditions suitable for nucleic acid amplification.

Also in accordance with the present invention, the multiple oligonucleotides species may comprise probes where each probe may be capable of hybridizing with the genetic material of a respiratory virus species. The sample is thus exposed with the probe under conditions suitable for hybridization.

As used herein the term “oligonucleotides species” means that an oligonucleotide has a nucleic acid sequence which is distinguishable from the nucleic acid sequence of other oligonucleotide.

One method which is currently used for amplifying genetic material is the polymerase chain reaction (PCR) or the reverse transcriptase polymerase chain reaction (RT-PCR). However, in some instances, the nucleic acids may be in a sufficient amount that amplification is not required.

In the present invention, four multiplex assays using a single-step RT-PCR method were designed for amplification and detection of nucleic acids from the above mentioned respiratory viruses. Combining the RT and the PCR in one step can conveniently prevent contamination. However optimization of reagents and conditions is usually required to enable efficient activities for both enzymes. It is to be understood herein that the separation of the amplification reactions into four multiplexes has been found to conveniently work. However, the amplification may be separated into more than four reactions. For example, although less convenient, viruses of multiplex 1, 2, 3 or 4 could be subdivided in 2, 3 or 4 distinct amplification reactions where relevant.

In the present invention, the PCR amplification for each multiplex can be performed using the same thermal cycling profile thereby allowing the amplification of all the nucleic acid targets at the same time (simultaneously) in a single apparatus (e.g., thermocycler).

Although nucleic acid amplification is often performed by PCR or RT-PCR, other methods exist. Non-limiting examples of such method include quantitative polymerase chain reaction (Q-PCR), ligase chain reaction (LCR), transcription-mediated amplification (TMA), self-sustained sequence replication (3SR), nucleic acid sequence-based amplification (NASBA), strand displacement amplification (SDA), recombinase polymerase amplification (RPA), loop-mediated isothermal amplification (LAMP), helicase-dependent amplification (HDA), helicase-dependent isothermal DNA amplification (tHDA), branched DNA (bDNA), cycling probe technology (CPT), solid phase amplification (SPA), rolling circle amplification technology (RCA), real-time RCA, solid phase RCA, RCA coupled with molecular padlock probe (MPP/RCA), aptamer based RCA (aptamer-RCA), anchored SDA, primer extension preamplification (PEP), degenerate oligonucleotide primed PCR (DOP-PCR), sequence-independent single primer amplification (SISPA), linker-adaptor PCR, nuclease dependent signal amplification (NDSA), ramification amplification (RAM), multiple displacement amplification (MDA), real-time RAM, and whole genome amplification (WGA) (Westin, L. et al., 2000, Nat. Biotechnol. 18:199-204 ; Notomi, T. et al., 2000, Nucleic Acids Res. 28:e63 ; Vincent, M. et al., 2004, EMBO reports 5:795-800 ; Piepenburg, O. et al., 2006, PLoS Biology 4:E204 ; Yi, J. et al., 2006, Nucleic Acids Res. 34:e81 ; Zhang, D. et al., 2006, Clin. Chim. Acta 363:61-70 ; McCarthy, E. L. et al., 2007, Biosens. Biotechnol. 22:126-1244 ; Zhou, L. et al., 2007, Anal. Chem. 79:7492-7500 ; Coskun, S. and Alsmadi, O., 2007, Prenat. Diagn. 27:297-302 ; Biagini, P. et al., 2007, J. Gen. Virol. 88:2629-2701 ; Gill, P. et al., 2007, Diagn. Microbiol. Infect. Dis. 59:243-249 ; Lasken, R. S. and Egholm, M., 2003, Trends Biotech. 21:531-535).

It should also be understood herein that the scope of this invention is not limited to a specific detection technology. Classically, detection of amplified nucleic acids is performed by standard ethidium bromide-stained agarose gel electrophoresis. Briefly, 10 μL of the amplification mixture were resolved by electrophoresis in a 2% agarose gel containing 0.25 μg/mL of ethidium bromide. The amplicons are then visualized under a UV transilluminator. Amplicon size is estimated by comparison with a molecular weight ladder. It is however clear that other method for the detection of specific amplification products, which may be faster and more practical for routine diagnosis, may be used. Such methods may be based on the detection of fluorescence after or during amplification.

One simple method for monitoring amplified DNA is to measure its rate of formation by measuring the increase in fluorescence of intercalating agents such as ethidium bromide or SYBR® Green I (Molecular Probes). If a more specific detection is required, fluorescence-based technologies can monitor the appearance of a specific product during the nucleic acid amplification reaction. The use of dual-labelled fluorogenic probes such as in the TaqMan™ system (Applied Biosystems) which utilizes the 5′-3′ exonuclease activity of the Taq polymerase is a good example (Livak K. J. et al., 1995, PCR Methods Appl. 4:357-362). TaqMan™ can be performed during amplification and this “real-time” detection is performed in a closed vessel hence eliminating post-PCR sample handling and consequently preventing the risk of amplicon carryover.

Several other fluorescence-based detection methods can be performed in real-time. Examples of such fluorescence-based methods include the adjacent hybridization probes (Wittwer, C. T. et al., 1997, BioTechniques 22:130-138), molecular beacon probes (Tyagi S. and Kramer F. R. 1996. Nat. Biotech. 14:303-308) and scorpion probes (Whitcomb et al., 1999, Nat. Biotech. 17:804-807). Adjacent hybridization probes are usually designed to be internal to the amplification primers. The 3′ end of one probe is labelled with a donor fluorophore while the 5′ end of an adjacent probe is labelled with an acceptor fluorophore. When the two probes are specifically hybridized in closed proximity (spaced by 1 to 5 nucleotides) the donor fluorophore which has been excited by an external light source emits light that is absorbed by a second acceptor that emit more fluorescence and yields a fluorescence resonance energy transfer (FRET) signal. Molecular beacon probes possess a stem-and-loop structure where the loop is the probe and at the bottom of the stem a fluorescent moiety is at one end while a quenching moiety is at the other end. The molecular beacons undergo a fluorogenic conformational change when they hybridize to their targets hence separating the fluorochrome from its quencher. The FRET principle has been used for real-time detection of PCR amplicons in an air thermal cycler equipped with a built-in fluorometer (Wittwer, C. T. et al. 1997, BioTechniques 22:130-138). Apparatus for real-time detection of PCR amplicons are capable of rapid PCR cycling combined with either fluorescent intercalating agents such as SYBR® Green I or FRET detection. Methods based on the detection of fluorescence are particularly promising for utilization in routine diagnosis as they are very simple, rapid and quantitative.

Exemplary embodiments of amplification conditions are provided in Example section. However, as used herein the term “amplification condition” refers to temperature and/or incubation time suitable to obtain a detectable amount of the target. Therefore, the term “similar amplification conditions” means that the assay may be performed, if desired, under similar temperature for each target. The term “similar amplification conditions” also means that the assay may be performed, if desired, under similar incubation time for each target. The term “similar amplification conditions” may in some instances also refer to the number of amplification cycles. However, it is well known in the art that number of cycles is not always critical. For example, some samples may be removed before others or left for additional amplification cycles. In other instances. The term “similar amplification conditions” may also refer to the nature of buffer and amplification reagents used (enzyme, nucleotides, salts, etc.). The term “similar amplification conditions” also means that the conditions (e.g., time, buffer, number of cycles, temperature, etc.) may be varied slightly or may be the same.

Exemplary embodiments of detection conditions are provided in the Example section. However, as used herein, the term “detection condition” refers to temperature and/or incubation time suitable to obtain a detectable signal (e.g., fluorescence emission, emission spectra, etc.) or other parameters suitable to obtain a detectable signal. The term “similar detection conditions” also means that the conditions may be varied slightly or may be the same.

Exemplary embodiments of hybridization conditions are provided in the Example section. As used herein, the term “similar hybridization conditions” means that the hybridization assay may be performed, if desired, under similar temperature for each target. The term “similar hybridization conditions” also means that the assay may be performed, if desired, under similar incubation time for each target. The term “similar hybridization conditions” may also refer to the nature of the hybridization solution used (salts, stringency etc.). The term “similar hybridization conditions” also means that the conditions (e.g., time, solution, temperature, etc.) may be varied slightly or may be the same.

Detection and identification of pathogens may be performed by sequencing. Simultaneous amplification and detection of nucleic acid material may also be performed using real-time PCR. Detection in liquid assays or solid phase assays (chips, arrays, beads, films, membranes, etc.) is also encompassed herewith.

Amplicon detection may thus be performed by hybridization using species-specific internal probes capable of binding to a desired amplification product. Such probes may be designed to specifically hybridize to amplicons amplified with the primers described herein. The probes may be labelled with biotin, digoxigenin or with any other reporter molecule. In an exemplary embodiment, the primers described in the present invention may be labelled with a fluorophore or dye including without limitation Cy3, Cy5, etc.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so.

Typically hybridization of the target nucleic acid is performed under moderate to high stringency conditions. Such high stringency conditions allow a higher specificity of the interaction between the probe and target. Hybridization may be performed at room temperature (19-25° C.) using probes attached to a solid support and hybridization solution containing amplicons. Active hybridization may be achieved using a microfluidic device, where the hybridization solution containing the amplicon are flowed above the microarray. Washing step may be performed with solutions allowing hybridization at varying stringencies. The microfluidic version of the procedure is typically performed within 15 min including the washing and rinsing steps. A person of skill in the art is well aware that nucleic acid hybridization and washing conditions can be modified and still achieve comparable levels of sensitivity and specificity as long as the overall process results in comparable stringency for nucleic acid recognition.

Probes (i.e., capture probes) targeting internal regions of the PCR amplicons generated using the amplification primer sets described above were therefore designed.

These capture probes were selected using multiple sequence alignments on the basis of optimal requirements previously described regarding sequence composition of the probe and its localization on the amplicon (Peytavi et al., 2005, Clin. Chem. 39:89-96). To cover all or most strains of a target viral species or genus, several probes have been designed for the ubiquitous species-specific/genus-specific detection of the target viruses.

These capture probes can be used either for real-time PCR detection (e.g. TaqMan probes, molecular beacons), for solid support hybridization (e.g. microarray hybridization, magnetic bead-based capture of nucleic acids, hydridization on membranes) or else.

Exemplary embodiments of probes have been provided in Table 2. However, a person of skill in the art will understand that other probes may be designed to detect the PCR amplicons generated using the primer pairs of Table 2 although with various efficiency or specificity. As such, the identity of the probe is not limited to the list provided herein or particularly provided in Table 2 but also extend to any probe which may be capable of specific binding with other regions of the PCR amplicon, including the sense or antisense strand of the PCR amplicon.

Microarrays of oligonucleotides represent a technology that is highly useful for multiparametric assays and which is encompassed by the present invention. Available low to medium density arrays (Heller M. J. et al., pp 221-224, In: Harrison, D. J., and van den Berg, A., 1998, Micro total analysis systems '98, Kluwer Academic Publisher, Dordrecht) could specifically capture fluorescent-labelled amplicons. Detection methods for hybridization are not limited to fluorescence; potentiometry, colorimetry and plasmon resonance are some examples of alternative detection methods. In addition to detection by hybridization, nucleic acid microarrays could be used to perform rapid sequencing by hybridization. Mass spectrometry could also be applicable for rapid identification of the amplicon or even for sequencing of the amplification products (Chiu N. H. and Cantor O. R., 1999, Clin. Chem. 45:1578; Berkenkamp S. et al., 1998, Science 281:260-262).

For the future of the assay format, integration of steps including sample preparation, genetic amplification, detection, and data analysis into a μTAS are also considered (Anderson, R. C. et al., pp. 11-16. In: Harrison, D. J., and van den Berg, A., 1998, Micro total analysis systems '98, Kluwer Academic Publisher, Dordrecht). In yet another embodiment, the probes described in this invention could be used without the need of prior PCR amplification. Promising ultra-sensitive detection technologies such as the use of polymeric biosensors based on the optical properties of the nucleic acid/polymer complex (Najari, A. et al., 2006, Anal. Chem. 78:7896-7899; Doré, K. et al., 2006, J. Fluoresc. 16:259-265; Ho, H. -A. et al., 2005 J. Am. Chem. Soc. 127:12673-12676 ; Doré, K. et al., 2004, J. Am. Chem. Soc. 126:4240-4244 ; Ho, H. -A. et al., 2002, Angew. Chem. Int. Ed. 41:1548-1551) could allow capture and detection of target pathogen species using hybridization probes, without the need for prior PCR amplification.

The present invention further relates to an array which may comprising a solid substrate (support) and a plurality of positionally distinguishable probes attached to or in association with the solid substrate (support). Each probe may comprise a different nucleic acid sequence and may be capable of specific binding to (i) a matrix gene of influenza A viruses (ii) a matrix gene of influenza B viruses, (iii) a nucleocapsid gene of human respiratory syncytial viruses, (iv) a nucleocapsid gene of human metapneumoviruses, (v) a 5′-non-coding region of human enteroviruses, (vi) a 5′-non-coding region of rhinoviruses, (vii) a fusion gene of parainfluenza viruses of type 1, (viii) a fusion gene of parainfluenza viruses of type 2, (ix) a fusion gene of parainfluenza viruses of type 3, (x) a fusion gene of parainfluenza viruses of type 4, (xi) a matrix gene of the coronaviruses OC43, (xii) a polymerase gene of the coronaviruses NL, (xiii) a polymerase gene of the coronaviruses 229E, (xiv) a polymerase gene of the coronaviruses SARS-CoV and (xv) a hexon region of the adenoviruses of serotypes associated with a respiratory infection. In accordance with the present invention, each of the probes may independently comprise 10 to 50 nucleotides.

In another aspect, the present invention provides an array which may comprise:
a) at least one member selected from the group consisting of an oligonucleotide comprising from 0 to 5 nucleotide addition and/or deletion to SEQ ID NO.:106, SEQ ID NO.:107, SEQ ID NO.:108, SEQ ID NO.:109, SEQ ID NO.:110, SEQ ID NO.:111, SEQ ID NO.:112, SEQ ID NO.:113, SEQ ID NO.:114, SEQ ID NO.:115, SEQ ID NO.:116, SEQ ID NO.:117, SEQ ID NO.:118, SEQ ID NO.:119, SEQ ID NO.:120, SEQ ID NO.:194, SEQ ID NO.:195, SEQ ID NO.:196 or a complement thereof and where the addition and/or deletion is located at a 5′ end and/or 3′ end of the nucleic acid sequence;
b) at least one member selected from the group consisting of an oligonucleotide comprising from 0 to 5 nucleotide addition and/or deletion to SEQ ID NO.:121, SEQ ID NO.:122, SEQ ID NO.:123, SEQ ID NO.:124, SEQ ID NO.:125, SEQ ID NO.:126, SEQ ID NO.:127, SEQ ID NO.:197, SEQ ID NO.:198, SEQ ID NO.:199 and SEQ ID NO.:200 or a complement thereof and where the addition and/or deletion is located at a 5′ end and/or 3′ end of the nucleic acid sequence;
c) at least one member selected from the group consisting of an oligonucleotide comprising from 0 to 5 nucleotide addition and/or deletion to SEQ ID NO.:128, SEQ ID NO.:129, SEQ ID NO.:130, SEQ ID NO.:131, SEQ ID NO.:132 and SEQ ID NO.:133 or a complement thereof and where the addition and/or deletion is located at a 5′ end and/or 3′ end of the nucleic acid sequence;
d) at least one member selected from the group consisting of an oligonucleotide comprising from 0 to 5 nucleotide addition and/or deletion to SEQ ID NO.:134, SEQ ID NO.:135, SEQ ID NO.:136, SEQ ID NO.:137, SEQ ID NO.:138, SEQ ID NO.:139, SEQ ID NO.:140, SEQ ID NO.:141, SEQ ID NO.:142, SEQ ID NO.:143, SEQ ID NO.144, SEQ ID NO.:145, SEQ ID NO.:146, SEQ ID NO.:147, SEQ ID NO.:148, SEQ ID NO.:149, SEQ ID NO.:150, SEQ ID NO.:151, SEQ ID NO.:152, SEQ ID NO.:201, SEQ ID NO.:202, SEQ ID NO.:203, SEQ ID NO.:204, SEQ ID NO.:205, SEQ ID NO.:206, SEQ ID NO.:207 and SEQ ID NO.:208 or a complement thereof and where the addition and/or deletion is located at a 5′ end and/or 3′ end of the nucleic acid sequence;
e) at least one member selected from the group consisting of an oligonucleotide comprising from 0 to 5 nucleotide addition and/or deletion to SEQ ID NO.:153 and SEQ ID NO.:154 or a complement thereof and where the addition and/or deletion is located at a 5′ end and/or 3′ end of the nucleic acid sequence;
f) at least one member selected from the group consisting of an oligonucleotide comprising from 0 to 5 nucleotide addition and/or deletion to SEQ ID NO.:155, SEQ ID NO.:156 and SEQ ID NO.:157 or a complement thereof and where the addition and/or deletion is located at a 5′ end and/or 3′ end of the nucleic acid sequence;
g) at least one member selected from the group consisting of an oligonucleotide comprising from 0 to 5 nucleotide addition and/or deletion to SEQ ID NO.:158, SEQ ID NO.:209 and SEQ ID NOs: 225 to 229 or a complement thereof and where the addition and/or deletion is located at a 5′ end and/or 3′ end of the nucleic acid sequence;
h) at least one member selected from the group consisting of an oligonucleotide comprising from 0 to 5 nucleotide addition and/or deletion to SEQ ID NO.:159 and SEQ ID NO.:160 or a complement thereof and where the addition and/or deletion is located at a 5′ end and/or 3′ end of the nucleic acid sequence;
i) at least one member selected from the group consisting of an oligonucleotide comprising from 0 to 5 nucleotide addition and/or deletion to SEQ ID NO.:161 or a complement thereof and where the addition and/or deletion is located at a 5′ end and/or 3′ end of the nucleic acid sequence;
j) at least one member selected from the group consisting of an oligonucleotide comprising from 0 to 5 nucleotide addition and/or deletion to SEQ ID NO.:162 or a complement thereof and where the addition and/or deletion is located at a 5′ end and/or 3′ end of the nucleic acid sequence;
k) at least one member selected from the group consisting of an oligonucleotide comprising from 0 to 5 nucleotide addition and/or deletion to SEQ ID NO.:163 and SEQ ID NO.:164 or a complement thereof and where the addition and/or deletion is located at a 5′ end and/or 3′ end of the nucleic acid sequence;
l) at least one member selected from the group consisting of an oligonucleotide comprising from 0 to 5 nucleotide addition and/or deletion to SEQ ID NO.:165, SEQ ID NO.:166, SEQ ID NO.:167, SEQ ID NO.:168, SEQ ID NO.:169, SEQ ID NO.:170, SEQ ID NO.:171, SEQ ID NO.:172, SEQ ID NO.:173, SEQ ID NO.:174, SEQ ID NO.:175, SEQ ID NO.:176, SEQ ID NO.:177, SEQ ID NO.:178 or a complement thereof and where the addition and/or deletion is located at a 5′ end and/or 3′ end of the nucleic acid sequence;
m) at least one member selected from the group consisting of an oligonucleotide comprising from 0 to 5 nucleotide addition and/or deletion to SEQ ID NO.:179, SEQ ID NO.:180, SEQ ID NO.:181, SEQ ID NO.:182, SEQ ID NO.:183, SEQ ID NO.:210, SEQ ID NO.:211, SEQ ID NO.:212, SEQ ID NO.:213, SEQ ID NO.:214 or a complement thereof and where the addition and/or deletion is located at a 5′ end and/or 3′ end of the nucleic acid sequence;
n) at least one member selected from the group consisting of an oligonucleotide comprising from 0 to 5 nucleotide addition and/or deletion to SEQ ID NO.:184, SEQ ID NO.:185, SEQ ID NO.:186, SEQ ID NO.:187, SEQ ID NO.:188 or a complement thereof and where the addition and/or deletion is located at a 5′ end and/or 3′ end of the nucleic acid sequence.

In accordance with the present invention, each oligonucleotide may be attached to or may be in association with a solid support. Further in accordance with the present invention, each oligonucleotide may be located at an addressable position.

The present invention also relates, in an additional aspect, to a library of oligonucleotides comprising at least two oligonucleotides described herein. In accordance with the present invention, each oligonucleotide may be provided in a separate container or may be attached to a solid support.

As mentioned above, aspects of the invention relates to a kit comprising the oligonucleotides described herein.

More particularly, the present invention relates to a kit which may comprise a plurality of oligonucleotides of from 10 to 50 nucleotides long capable of specific binding to a gene selected from the group consisting of (i) a matrix gene of the influenza A virus (ii) a matrix gene of the influenza B virus, (iii) a nucleocapsid gene of the human respiratory syncytial virus, (iv) a nucleocapsid gene of the human metapneumovirus, (v) a 5′-non-coding region of the human enterovirus, (vi) a 5′-non-coding region of the rhinovirus, (vii) a fusion gene of the parainfluenza virus type 1, (viii) a fusion gene of the parainfluenza virus type 2, (ix) a fusion gene of the parainfluenza virus type 3, (x) a fusion gene of the parainfluenza virus type 4, (xi) a matrix gene of the coronavirus 0043, (xii) a polymerase gene of the coronavirus NL, (xiii) a polymerase gene of the coronavirus 229E, (xiv) a polymerase gene of the coronavirus SARS-CoV and (xv) a hexon region of the adenovirus. In accordance with the present invention, each oligonucleotide of the plurality of oligonucleotides may be capable of binding to the gene of one respiratory virus species and not the gene of the other respiratory virus species.

In an embodiment of the invention, the kit may comprise a plurality of oligonucleotides for the specific amplification of human enteroviruses, rhinoviruses, respiratory syncytial viruses and metapneumoviruses. The plurality of oligonucleotides for the specific amplification of human enteroviruses, rhinoviruses, respiratory syncytial viruses and metapneumoviruses may be provided in separate containers each comprising individual oligonucleotides or each comprising a virus (gene) specific oligonucleotide primer pair. The plurality of oligonucleotides may also be provided in a single container comprising a mixture of oligonucleotides for amplification of each target gene.

In another embodiment, the kit may comprise a plurality of oligonucleotides for the specific amplification of the viruses influenza A, parainfluenza type 1, parainfluenza type 2 and parainfluenza type 3. The plurality of oligonucleotides for the specific amplification of the viruses influenza A, parainfluenza type 1, parainfluenza type 2 and parainfluenza type 3 may be provided in separate containers each comprising individual oligonucleotides or each comprising a virus (gene) specific oligonucleotide primer pair. The plurality of oligonucleotides may also be provided in a single container comprising a mixture of oligonucleotides for amplification of each target gene.

In an additional embodiment, the kit may comprise a plurality of oligonucleotides for the specific amplification of the coronaviruses SARS-CoV, 229E, NL and OC43. The plurality of oligonucleotides for the specific amplification of the coronaviruses SARS-CoV, 229E, NL and OC43 may be provided in separate containers each comprising individual oligonucleotides or each comprising a virus (gene) specific oligonucleotide primer pair. The plurality of oligonucleotides may also be provided in a single container comprising a mixture of oligonucleotides for amplification of each target gene.

In a further embodiment, the kit may comprise a plurality of oligonucleotides for the specific amplification of adenoviruses, influenza B and parainfluenza type 4. The plurality of oligonucleotides for adenoviruses, influenza B and parainfluenza type 4 may be provided in separate containers each comprising individual oligonucleotides or each comprising a virus (gene) specific oligonucleotide primer pair. The plurality of oligonucleotides may also be provided in a single container comprising a mixture of oligonucleotides for amplification of each target gene.

The kit may also comprise the plurality of oligonucleotides of two, three or four of the groups of viruses mentioned above.

In accordance with the present invention, the kit may comprise in a separate container or attached to a solid support, an oligonucleotide (e.g., a probe) for the detection of the target gene.

The present invention also relates in an additional aspect to a method for the diagnosis of a respiratory infection in an individual in need. The method may comprise detecting the presence or absence of a pathogen from a sample obtained from the individual with oligonucleotides capable of specific binding with a genetic material of a respiratory virus species selected from the group consisting of (i) an influenza A virus (ii) an influenza B virus, (iii) a human respiratory syncytial virus, (iv) a human metapneumovirus, (v) a human enterovirus, (vi) a rhinovirus, (vii) a parainfluenza virus type 1, (viii) a parainfluenza virus type 2, (ix) a parainfluenza virus type 3, (x) a parainfluenza virus type 4, (xi) a coronavirus OC43, (xii) a coronavirus NL, (xiii) a coronavirus 229E, (xiv) a coronavirus SARS-CoV and (xv) an adenovirus serotype associated with respiratory infections. In accordance with the present invention, the presence of the pathogen may be indicative of a respiratory infection associated with the detected pathogen.

In an embodiment of the invention, the presence or absence of the pathogen may be determined by detecting the genetic material from a respiratory virus species, such as, for example (i) a matrix gene of the influenza A virus (ii) a matrix gene of the influenza B virus, (iii) a nucleocapsid gene of the human respiratory syncytial virus, (iv) a nucleocapsid gene of the human metapneumovirus, (v) a 5′-non-coding region of the human enterovirus, (vi) a 5′-non-coding region of the rhinovirus, (vii) a fusion gene of the parainfluenza virus type 1, (viii) a fusion gene of the parainfluenza virus type 2, (ix) a fusion gene of the parainfluenza virus type 3, (x) a fusion gene of the parainfluenza virus type 4, (xi) a matrix gene of the coronavirus OC43, (xii) a polymerase gene of the coronavirus NL, (xiii) a polymerase gene of the coronavirus 229E, (xiv) a polymerase gene of the coronavirus SARS-CoV and/or (xv) a hexon region of the adenovirus and combination thereof.

The present invention is illustrated in further details by the following non-limiting examples.

### Example 1

### Specific Amplification and Detection of 15 Clinically Important Respiratory Viruses Using a Combination of PCR Primers in a Multiplex Format

RNA was extracted from viral cell culture supernatants using the Magazorb RNA extraction kit (Cortex, San Leandro, Calif.) and KingFisher ML instrument (Thermo Scientific, Waltham, Calif.). One μl of purified RNA was used for RT-PCR. The 20-μl PCR mixtures contained 0.6 μM each primer (SEQ ID Nos 78, 79, 80, 81, 82, 83, 84, 85, 86, 87, 88, 89, 90, 91, 92, 93, 94, 95, 96, 97, 98, 99, 100, 101, 102, and 103) separated in different multiplex as described below. Primers for the lysis control (SEQ ID Nos 104, and 105) were at 0.3 μM. RT-PCR was performed using the One-step RT-PCR kit (Qiagen). The viruses were obtained from the American Type Culture Collection (ATCC) as well as from respiratory tract clinical specimens obtained at the CHUL Pavilion of the Centre Hospitalier Universitaire de Québec (CHUQ).

PCR experiments were performed using the following thermal profile on a PTC-200 thermal cycler (Bio-Rad): a single cycle of reverse transcription for 30 min at 50° C. and then 15 min at 95° C. for reverse transcriptase inactivation and Taq DNA polymerase activation followed by 45 PCR cycles of 15 sec at 95° C. for denaturation, 10 sec at 54° C. for primer annealing, and 25 sec at 72° C. for the extension step. Four multiplex PCR assays have been developed for amplification and identification of the most clinically relevant respiratory viruses (Table 2). Multiplex 1 comprises primers for the detection and identification of human respiratory syncytial virus, human metapneumovirus, human rhinoviruses/enteroviruses (all serotypes), as well as primers for the amplification of the lysis control. Multiplex 2 comprises primers for the detection and identification of parainfluenzaviruses of type 1, 2, and 3, as well as influenza A. Multiplex 3 comprises primers for the detection and identification of coronaviruses NL, 229E, OC43, and SARS-CoV. Finally, multiplex 4 comprises primers for the detection and identification of all 7 serotypes of adenoviruses (1, 2, 3, 4, 5, 7, and 21) associated with respiratory infections in humans, parainfluenzavirus of type 4, and influenza B. Table 2 provides a list of selected PCR primers and target genes for each target virus. Amplification products were analysed by agarose gel electrophoresis as previously described (Ke, D., et al., 2000, Clin. Chem. 46:324-31).

### Evaluation of the Multiplex RT-PCR Assays

### Determination of the Analytical Sensitivities

Partial or complete target gene selected for each virus were PCR-amplified and cloned as follows. RT-PCR was performed as described above using the viruses described in Table 5. Amplicons were cloned directly after amplification using the TOPO-TA cloning kit (Invitrogen). Transformation into DH5α max efficiency (Invitrogen) led to the recovery of recombinant plasmids carrying an inserted PCR product. For each target gene, several clones containing inserted amplicons were sequenced to ensure the absence of sequencing errors attributable to nucleotide miscorporations by the Taq DNA polymerase. Sequence assembly was performed with the aid of the Sequencher 3.0 software (Gene Codes).

After confirming the accuracy of the inserted DNA by sequencing, the recombinant plasmids, containing the targeted portion of the gene of interest, were linearized and then transcribed using Ampliscribe T7-Flash kit (Epicentre) as specified by the manufacturer using 300 to 600 ng of plasmid DNA. The produced RNA was then purified using the RNA cleanup protocol from the Rneasy kit (Qiagen) or the Magazorb kit (Cortex) on a Kingfisher ML as specified by the manufacturer without the lysis step. Quantitation of RNA was performed by spectrophotometry at 260 nm using an Ultrospec 2000 (Pharmacia), and these data were then converted to copy number/μl. RNA transcripts were visualized on an Agilent Bioanalyzer to verify the absence of degradation (RNA 6000 Nano LabChip® Kit). These transcripts were used to determine the analytical sensitivity of each multiplex assay by spiking each PCR reaction with a known amount of transcript corresponding to the targeted portion of each gene. The detection limit for the 15 respiratory viruses ranged from between 50 to 100 copies of viral genome per RT-PCR reaction depending on the target gene (Table 2).

### Determination of the Assay Specificity and Ubiquity

For each target virus, the specificity of the RT-PCR assays was verified using highly concentrated viral RNA extracted from a cell culture, as well as the equivalent of 10000 genome copies of human genomic DNA, and ˜30000 genome copies of bacteria commonly associated with respiratory infections (i.e. Streptococcus pneumoniae, Moraxella catharrhalis, Haemophilus influenzae, and Legionella pneumophila). Non-specific RT-PCR amplification products were observed on an ethidium bromide-stained agarose gel when human DNA was used as template. Similarly, non-specific amplification was also observed with genomic RNA/DNA from other respiratory viruses. On the other hand, non-specific amplification products were not observed with genomic DNA from the selected bacteria used as template. The ubiquity of the assay was verified using the equivalent of 100 viral genome copies from 2 strains of enteroviruses, 1 strain of coxsackievirus, 2 strains of rhinovirus, 3 strains of RSV, 3 strains of hMPV, 4 strains of influenza A, 8 strains of influenza B, 5 strains of parainfluenzavirus of type 1, 2 strains of parainfluenzavirus of type 2, 2 strains of parainfluenzavirus of type 3, 8 strains of parainfluenzavirus of type 4, and 8 strains of adenoviruses. For all target species, all strains of a given viral species were amplified and detected efficiently with the multiplex RT-PCR assays targeting this species.

The multiplex RT-PCR assays, which are objects of the present invention, allowed the sensitive and ubiquitous amplification of the 15 most clinically important respiratory viruses when coupled with standard agarose gel electrophoresis for amplicons detection.

### Example 2

### Simultaneous Detection of 15 Respiratory Viruses on a Microarray Chip

Typically, double-stranded amplification products are denatured at 95° C. for 1 to 5 min, and then cooled on ice prior to hybridization. Since double-stranded amplicons tend to reassociate with their complementary strand instead of hybridizing with the probes, single-stranded amplicons may advantageously be used for hybridization. One such method to produce single-stranded amplicons is to digest one strand with the exonuclease from phage Lambda. Preferential digestion of one strand can be achieved by using a 5-prime phosphorylated primer for the complementary strand and a fluorescently-labelled primer for the target strand (Boissinot et al. 2007, Clin. Chem., 53:2020-3). Briefly, amplicons generated with such modified primers were digested by adding 10 units of Lambda exonuclease (New-England Biolabs) directly to PCR reaction products and incubating them at 37° C. for 5 min. Such digested amplification products can readily be used for microarray hybridization without any prior heat treatment.

Microarrays are typically made by pinspotting oligonucleotide probes onto a glass slide surface but the person skilled in the art knows that other surfaces and other methods to attach probes onto surfaces exist and are also covered by the present invention. Lateral flow microarrays represent an example of recent rapid solid support hybridization technology (Carter and Cary, 2007, Nucl. Acids Res. 35:e74). For the illustrative example described below, oligonucleotide probes modified with a 5′ amino-linker were suspended in Microspotting solution plus (TeleChem International) and spotted at 30 μM on Super Aldehyde slides (Genetix) using a VIRTEK SDDC-2 Arrayer (Bio-Rad Laboratories). In addition to DNA or RNA oligonucleotides, nucleotide analogs such as peptide nucleic acids (PNA), locked nucleic acids (LNA) and phosphorothioates can be used as probes and are also the object of this invention.

All hybridization experiments were carried out using amplification products digested with Lambda exonuclease as described above. Primers (SEQ 78, 79, 80, 81, 82, 83, 84, 85, 86, 87, 88, 89, 90, 91, 92, 93, 94, 95, 96, 97, 98, 99. 100, 101, 102, and 103,) were used for the RT-PCR step. Capture probes used for microarray hybridization with each of the four RT-PCR multiplex assays are described in Table 2. The positive control in all hybridization experiments consisted of hybridizing an amino-modified 20-mer oligonucleotide with its Cy3-labelled 20-mer complementary strand. Passive hybridizations were carried out with 4.8 μL of PCR-amplified reaction into 15.2 μl hybridization solution containing 6× SSPE, 0,03% PVP, 30% formamide+5 nM of Cy3-bbc1, for one hour at room temperature (e.g., 19-25° C.). Slides were subsequently washed for 5 min with 2× SSPE buffer (OmniPur EMD) containing 0.1% SDS followed by rinsing with a solution of 2× SSPE buffer for 5 min. Microfluidic hybridizations were performed as previously described (Peytavi et al., 2005, 39:89-96). Fluorescent images of all slides were obtained with a ScanArray 4000 XL microarray scanner (GSI Lumonics/Packard Biochips, Billerica, Mass.) and data analysis was performed with GenePix Pro 6.0 (Molecular Devices).

The microfluidic version of the procedure can be performed within 15 min including the wash and rinse steps. A person of skill in the art is well aware that nucleic acid hybridization and washing conditions can be modified and still achieves comparable levels of sensitivity and specificity as long as the overall process results in comparable stringency for nucleic acid recognition.

An advantage of the present invention that all microarray hybridizations and washing procedures may be performed under uniform conditions for all probes and multiplex amplification combinations.

The determination of the assay sensitivity was performed using serial dilutions of RNA transcripts from each target gene cloned into plasmids as described in Example 1. Each PCR reaction was spiked with a known amount of target gene, amplified, and hybridized onto the microarray. Analytical sensitivities with the microarray detection was equivalent to that obtained with detection using ethidium bromide-stained gels (i.e. the detection limit for the 15 respiratory viruses ranged from 50 to 100 copies of viral genome depending on the target gene (Table 2).

The specificity of the assay was verified as described in Example 1. After amplification by RT-PCR, the PCR reactions were prepared for hybridization onto the microarray of oligonucleotide capture probes. Even though Example 1 demonstrated that RT-PCR amplification of human and virus nucleic acid materials generated non-specific amplicons, there was no cross-hybridization observed on the microarray. The ubiquity of the assay was verified using the equivalent of 100 viral genome copies from different strains of each target viral species as described in Example 1. For all target species, all strains of a given viral species were amplified and detected efficiently with the multiplex RT-PCR assays targeting this species.

Capture probes for microarray hybridization, which are objects of the present invention, allowed specific, sensitive, and ubiquitous detection of amplicons generated by RT-PCR from the 15 most clinically important respiratory viruses.

### Example 3

### Evaluation of the Multiplex PCR Assay with Clinical Respiratory Specimens from Patients

Nasopharyngeal aspirates (NPA) from children of less than 3 years old were collected and frozen in aliquots until the beginning of the study. The criterion for selection of patients into the clinical study was a medical consultation for respiratory illness symptoms where the clinician requested a rapid immunologic diagnostic test (BINAX) for Influenza A and B and/or RSV. The genetic material from the NPA sample was extracted and purified using the following procedure: 850 μl of guanidium thiocyanate (GT) (4.5 M) was added into a 1.5 ml tube containing 0.005 g of silica beads. Subsequently, 200 μl of NPA specimen was added to the tube and mixed by inversion for 10 minutes. The GT solution allows to lyse the viruses potentially present in a NPA sample, and to bind the released nucleic acids to the silica beads. The tube was then centrifuged at 10000 g for 1 minute and the supernatant was removed. The pelleted beads were treated with 750 μl of ethanol 70% (prepared with DEPC water) to wash the nucleic acids, and then, the tube was centrifuged again at 10000 g for 1 minute. After removing the supernatant, 30 μl of DEPC water containing 1 μl of RNasin (Promega) was added to the pellet and incubated for 10 minutes at 60° C. to release nucleic acids. The whole content of the tube was then transferred to a Spin-X column (0.22 μM filter, Fisher Scientific) and purified genetic material was eluted from the column according to their protocol. Amplification and detection of the genetic material were performed as described in Examples 1 and 2.

A total of 134 NPA specimens were tested. From these, 106 were RSV-positive based on nucleic acid testing. In comparison, only 89 of the 134 NPA specimens were positive for RSV based on parallel testing with the BINAX assay. Other detected viruses include enteroviruses/rhinoviruses (n=18), coronavirus OC43 (n=10), adenoviruses (n=9), PIV-3 (n=3), PIV-4 (n=3), hMPV (n=3), and influenza A (n=1) and B (n=2). Eight specimens were negative for all tested viruses.

The molecular assay described in Examples 1 and 2 allowed rapid and sensitive detection from NPA specimens of respiratory viruses. It should be noted that some of the undetected viruses have a relatively low prevalence in the population (e.g. coronavirus responsible for SARS-CoV) and it is not surprising that we have not found any in the clinical specimen tested.

We believe that the present invention has provided reagents capable of reaching the goals of specificity, sensitivity and uniformity for the identification of 15 respiratory virus species. These reagents may meet the conditions needed for detection on solid support.

Although the present invention has been described herein by way of exemplary embodiments, it can be modified without departing from the scope and the nature of the invention.

The present description refers to a number of documents, the content of which is herein incorporated by reference in their entirety.

TABLE 1
List of target gene sequences used for the design of PCR primers and
hybridization probes for the detection of 15 respiratory viruses.

| Respiratory Virus | Target Gene | Identification* | SEQ ID NOs |
|---|---|---|---|
| Influenza A | Matrix | CCRI-15830 | 10 |
| | | CCRI-15707 | 9 |
| | | CCRI-15634 | 8 |
| | | CCRI-15615 | 7 |
| | | CCRI-15592 | 6 |
| | | CCRI-15591 | 5 |
| | | CCRI-15590 | 4 |
| | | CCRI-15589 | 3 |
| | | CCRI-15587 | 2 |
| | | CCRI-15586 | 1 |
| | | CCRI-15588 | 11 |
| | | CCRI-16240 | 13 |
| | | CCRI-16130 | 12 |
| | | CCRI-16241 | 14 |
| | | u65575; u65569; dq009919; ay947475; | |
| | | y651387; af398876; af138719; af115287; | |
| | | af038274; af038272; af038271; | |
| | | ay611525ii; ay210270ii; ay210269ii; | |
| | | ay210268ii; ay210267ii; ay210266ii; | |
| | | ay210265ii; ay210264ii; ay210263ii; | |
| | | ay210262ii; ay210261ii; ay210260ii; | |
| | | ay210259ii; ay210258ii; ay210257ii; | |
| | | ay210256ii; ay210255ii; ay210254ii; | |
| | | ay210253ii; ay210252ii; ay210251ii; | |
| | | ay210250ii; ay210249ii; ay210248ii; | |
| | | ay210247ii; ay210246ii; ay210245ii; | |
| | | ay210244ii; ay210243ii; ay210242ii; | |
| | | ay210241ii; ay210240ii; ay210239ii; | |
| | | ay210065ii; ay210064ii; ay210063ii; | |
| | | ay210062ii; ay210061ii; ay210060ii; | |
| | | ay210059ii; ay210058ii; ay210057ii; | |
| | | ay210056ii; ay210055ii; ay210054ii; | |
| | | ay210053ii; ay210052ii; ay210051ii; | |
| | | ay210050ii; ay210049ii; ay210048ii; | |
| | | ay210047ii; ay210046ii; ay210045ii; | |
| | | ay210044ii; ay210043ii; ay210042ii; | |
| | | ay210041ii; ay210040ii; ay210039ii; | |
| | | ay210038ii; ay210037ii; ay210036ii; | |
| | | ay210035ii; ay210034ii; ay210033ii; | |
| | | ay210032ii; ay210031ii; ay210030ii; | |
| | | ay210029ii; ay210028ii; ay210027ii; | |
| | | ay210026ii; af255374ii; af255373ii; | |
| | | af255372ii; af255371ii; af255370ii; | |
| | | af255369ii; af255368ii; af255367ii; | |
| | | af255366ii; af255365ii; af255364ii; | |
| | | af255363ii; af115287i; af115286i; | |
| | | af084284i; af084283i; af084282i; | |
| | | af046090i; af046082i; ay043025; | |
| | | ay043025ii; cy000250; af255382; | |
| | | af255384; af255383; af255381; | |
| | | ay626144; ay627887; ay627893; | |
| | | AY646079; AY611525; ay648288; | |
| | | ay650271; ay849792; ay575903; | |
| | | ay575902; ay575899; ay509042; | |
| | | ay509041; ay221538; ay059512; | |
| | | ay059511; ay221531; ay221530; | |
| | | ay509058; ay509049; ay509052; | |
| | | ay509050; ay509044; ay509043; | |
| | | ay509040; ay509064; ay509062; | |
| | | ay221533; ab212283; ay221532; | |
| | | 21326688; 28849446; 47716778; | |
| | | 70955556; 70955553; 71025303; | |
| | | 71025294; 70955559; 70955550; | |
| | | 70955547; 70955544; 70955541; | |
| | | 70955538; 70955535; 70955532; | |
| | | 70955529; 70955526; 70955523; | |
| | | 70955520; 70955517; 71025300; | |
| | | 71025297; 70955562; 70955565; | |
| | | 50234713; 50234710; 58618457; | |
| | | 50234668; 71370762; 71370741; | |
| | | 71370720; 71370717; 71370711; | |
| | | 54873471; 71370750; 71370714; | |
| | | 71370759; 71370756; 71370753; | |
| | | 71370687; 71370675; 58618459; | |
| | | 50234656; 50234665; 50234662; | |
| | | 50234659; 50234653; 50234650; | |
| | | 50234671; 71370765; 71370660; | |
| | | 50234620; 71370744; 71370735; | |
| | | 71370732; 71370729; 71370723; | |
| | | 71370708; 71370705; 71370702; | |
| | | 71370696; 71370693; 71370684; | |
| | | 71370678; 51094107; 50234635; | |
| | | 50234632; 50234623; 50234629; | |
| | | 50234626; 71370726; 71370669; | |
| | | 71370738; 71370666; 71370657; | |
| | | 71370747; 71370690; 71370681; | |
| | | 71370672; 71370663; 71370654; | |
| | | 71370699; 50234617; 57916086; | |
| | | 50234701; 50234758; 50234746; | |
| | | 50234743; 50234740; 57915999; | |
| | | 50234716; 50234689; 56548889; | |
| | | 47834913; 47834911; 56548887; | |
| | | 50234725; 50234692; 50234755; | |
| | | 41207454; 57916038; 58531180; | |
| | | 58531162; 58531144; 58531126; | |
| | | 58531094; 56548891; 56548893; | |
| | | 50234737; 50234614; 50234599; | |
| | | 50234605; 50234602; 50234698; | |
| | | 50234611; 50234608; 50234728; | |
| | | 50234695; 47834915; 55233222; | |
| | | 50956633; 50234722; 47834917; | |
| | | 50234719; 50234707; 47834923; | |
| | | 70955570; 50234686; 47834921; | |
| | | 50234752; 50234749; 50234731; | |
| | | 50234734; 70955568; 50234704; | |
| | | 28849450; 50234683; 50234674; | |
| | | 50234677; 58374191; 21359671; | |
| | | 5805288; 28812259; 9863926; | |
| | | 9863908; 21326676; 9863871; | |
| | | 19697823; 19697821; 18092191; | |
| | | 18092189; 9863889; 19697827; | |
| | | 19697825; 19697829; 28849422; | |
| | | 28812263; 28849438; 28849412; | |
| | | 28849442; 28849428; 28849426; | |
| | | 28849414; 28849440; 28849432; | |
| | | 28849430; 28849416; 28849434; | |
| | | 28849410; 13447384; 28812265; | |
| | | 28812261; 50365714; 6048814; | |
| | | 6048811; 6048802; 6048808; | |
| | | 6048790; 3335406; 6048799; | |
| | | AF098562; AF098561; AY651401; | |
| | | isdn140089; isdn133365; isdn140088; | |
| | | isdn140092; dq643814; cy014506; | |
| | | cy014445; cy014369; isdn137455; | |
| | | cy014485; cy014478; isdn209856; | |
| | | cy013798; cy008621; ab036778; | |
| | | ay835670; ay835671; ay836589; | |
| | | cy002057; cy002441; cy002185; | |
| | | cy002217; cy002970; cy002505; | |
| | | cy002713; cy002209; cy002233; | |
| | | cy002433; cy002449; cy002481; | |
| | | cy002729; cy002177; cy003073; | |
| | | cy002249; cy002281; cy002955; | |
| | | cy003049; cy002193; cy002225; | |
| | | cy002241; cy002265; cy002417; | |
| | | cy002489; cy002609; cy002761; | |
| | | cy002769; cy003033; cy002201; | |
| | | cy002425; cy002041; | |
| | | cy002721; cy002465; cy002737; | |
| | | cy002025; cy002257; cy002033; | |
| | | cy002049; cy002065; cy002457; | |
| | | cy002081; cy002289; cy002585; | |
| | | cy002923; cy002931; cy002939; | |
| | | cy002963; cy002977; cy003041; | |
| | | cy002409; cy002947; cy002601; | |
| | | cy002907; cy003057; cy002593; | |
| | | cy002073; cy002017; cy002915 | |
| human respiratory | nucleocapsid | CCRI-15958 | 16 |
| syncytial virus | | CCRI-15552 | 17 |
| | | CCRI-15959 | 18 |
| | | CCRI-15957 | 19 |
| | | CCRI-15595 | 20 |
| | | ay151194; ay151195; ay151196; | |
| | | ay151197; ay151198; ay151199; | |
| | | ay151200; ay151201; ay151202; | |
| | | ay151203; ay151204; ay151205; | |
| | | ay151206; ay151207; ay151207; | |
| | | ay151203; ay151204; ay151201; | |
| | | ay151202; ay151200; ay151206; | |
| | | d00736; ay151205; m74568; | |
| | | m11486; ay151199; ay151194; | |
| | | ay151196; ay151198; ay151197; | |
| | | ay151195; x00001; af013254; | |
| | | u39662 | |
| human | | CCRI-15597 | 22 |
| metapneumovirus | nucleocapsid | ay575719; ay145278; ay145276; | |
| | | ay145272; ay321502; ay321504; | |
| | | ay321505; ay145272; ay145276; | |
| | | n_ay145278; n_ay575719; ay550156; | |
| | | ay550155; ay550154; ay550153; | |
| | | ay550152; ay550151; ay550150; | |
| | | ay550149; ay550148; ay340102; | |
| | | ay340101; ay340100; ay340099; | |
| | | ay340098; ay340097; ay340096; | |
| | | ay340095; ay340094; ay340093; | |
| | | ay158465; ay158464; ay158463; | |
| | | af371337; ay158463; ay550151; | |
| | | ay550156; ay158464; ay158465; | |
| | | ay550150; ay550155; ay550148; | |
| | | ay550149; ay550152; ay550153; | |
| | | ay550154; ay340093; ay340094; | |
| | | ay340095; ay340096; ay340097; | |
| | | ay340098; ay340099; ay340100; | |
| | | ay340101; ay340102; af443831; | |
| | | af443830; ay321501; ay321503; | |
| | | af443830; af443831; ay575718; | |
| | | ay145273; ay145274; ay145275; | |
| | | ay145279; ay145280; ay145281; | |
| | | ay145282; ay145283; ay145284; | |
| | | ay145285; ay145286; ay575718; | |
| | | ay145286; ay145285; ay145284; | |
| | | ay145282; ay145281; ay145280; | |
| | | ay145279; ay145277; ay145277; | |
| | | ay145275; ay145274; ay145273; | |
| | | ay145283 | |
| Enterovirus | 5′non-coding | CCRI-15531 | 23 |
| | | CCRI-15527 | 24 |
| | | CCRI-15515 | 25 |
| | | CCRI-15502 | 26 |
| | | CCRI-15883 | 27 |
| | | CCRI-15869 | 28 |
| | | CCRI-15872 | 29 |
| | | CCRI-15840 | 30 |
| | | CCRI-15882 | 31 |
| | | CCRI-15850 | 32 |
| | | CCRI-15561 | 33 |
| | | CCRI-15734 | 34 |
| | | CCRI-15732 | 35 |
| | | CCRI-15733 | 36 |
| | | coxa2_af303036; coxa2_af412341; | |
| | | coxa3_af303037; coxa3_af412342; | |
| | | coxa5_af303044; coxa5_af412343; | |
| | | coxa6_af412344; coxa7_af412345; | |
| | | coxa7_af068879; coxa10_af412346; | |
| | | coxa12_af412348; coxa12_af303042; | |
| | | coxa12_af303041; coxa13_af303040; | |
| | | coxa14_af412349; coxa15_af412350; | |
| | | coxa17_af412351; coxa17_af412347; | |
| | | coxa18_af412354; coxa18_af412352; | |
| | | coxa18_af303038; coxa19_af412353; | |
| | | coxa20_af303039; coxa21_af076999; | |
| | | coxa21_af068880; coxa21_af412355; | |
| | | coxa21_aj001339; coxa22_af412356; | |
| | | coxa24_af068881; coxb1_ay373044; | |
| | | coxb1_ay373038; coxb1_ay373041; | |
| | | coxb1_ay373039; coxb1_ay373040; | |
| | | coxb1_ay373037; coxb1_ay373042; | |
| | | coxb1_ay373043; coxb1_ay373045; | |
| | | coxb1_ay373036; coxb1_s76767; | |
| | | coxb2_ay373052; coxb2_ay373046; | |
| | | coxb2_ay373057; coxb2_ay373055; | |
| | | coxb2_ay373054; coxb2_ay373058; | |
| | | coxb2_ay373051; coxb2_ay373050; | |
| | | coxb2_ay373053; coxb2_ay373049; | |
| | | coxb2_ay373056; coxb2_ay373048; | |
| | | coxb2_ay373047; coxb2_af225474; | |
| | | coxb2_af225473; coxb3_ay373063; | |
| | | coxb3_ay373062; coxb3_ay373061; | |
| | | coxb3_ay373064; coxb3_ay373059; | |
| | | coxb3_ay373066; coxb3_ay373067; | |
| | | coxb3_ay373060; coxb4_ay373076; | |
| | | coxb4_ay373073; coxb4_ay373075; | |
| | | coxb4_ay373074; coxb4_ay373072; | |
| | | coxb4_ay373069; coxb4_ay373068; | |
| | | coxb4_ay373070; coxb4_ay373071; | |
| | | coxb4_ay373084; coxb5_ay373079; | |
| | | coxb5_ay373089; coxb5_ay373088; | |
| | | coxb5_ay373087; coxb5_ay373082; | |
| | | coxb5_ay373065; coxb5_ay373086; | |
| | | coxb5_ay373085; coxb5_ay373083; | |
| | | coxb5_ay373081; coxb5_ay373080; | |
| | | coxb5_ay373078; coxb6_af225476; | |
| | | coxb6_af225475; coxb4_ay373077; | |
| | | coxb6_ay373090; coxb6_af225478; | |
| | | coxb6_af225477; echo4_af447482; | |
| | | echo6_af447481; echo11_af447484; | |
| | | echo11_af447485; echo11_af447486; | |
| | | eho11_af447487; echo11_af447472; | |
| | | echo11_af447474; echo11_af447475; | |
| | | echo11_af447476; echo11_af447477; | |
| | | echo19_af447479; echo19_af447480; | |
| | | echo19_af447483; echo30_s76768; | |
| | | echo30_s76769; entero75_ay556070; | |
| | | entero74_ay556057; entero90_ay773285; | |
| | | enteroca55_af241359; entero11_af068885; | |
| | | entero69_ay302560; entero69_af412376; | |
| | | entero70_nc_001430; enteroe71_u00872; | |
| | | entero71_ab183003; entero71_ay465356; | |
| | | rhino1a_af108179; rhino6_af542425; | |
| | | rhino7_af108185; rhino13_af542452; | |
| | | rhino17_af542419; rhino21_af108180; | |
| | | rhino27_af542449; rhino29_af108181; | |
| | | rhino37_af108182; rhino43_af542450; | |
| | | rhino45_af542451; rhino51_af542422; | |
| | | rhino52_af542423; rhino59_af542424; | |
| | | rhino58_af108183; rhino62_af108184; | |
| | | rhino69_af542426; rhino70_af542427; | |
| | | rhino72_af108186; rhino84_af542429; | |
| | | rhino86_af542431; rhino87_ay062273; | |
| | | rhino87_af108187; rhino91_af542432; | |
| | | rhino92_af108149; rhino93_af108154; | |
| | | rhino93_af108155; rhino93_af108151; | |
| | | rhino93_af108152; rhino93_af108150; | |
| | | rhino94_af108161; rhino94_af108159; | |
| | | rhino94_af108157; rhino94_af108158; | |
| | | rhino94_af108156; rhino94_af108160; | |
| | | rhino95_af108176; rhino95_af108175; | |
| | | rhino95_af108174; rhino95_af108167; | |
| | | rhino95_af108163; rhino95_af108164; | |
| | | rhino95_af108170; rhino95_af108173; | |
| | | rhino95_af108162; rhino95_af108169; | |
| | | rhino93_af108153; rhino95_af108172; | |
| | | rhino95_af108171; rhino95_af108165; | |
| | | rhino95_af108178; rhino95_af108166; | |
| | | rhino95_af108168; rhino95_af108177 | |
| PIV-1 | fusion | CCRI-15598 | 38 |
| | | CCRI-15560 | 39 |
| | | af016279; af016279i; af457102; af016279; | |
| | | af457102i; m22347 | |
| | | CCRI-15838 | 40 |
| PIV-2 | Fusion | CCRI-15641 | 42 |
| | | CCRI-15839 | 43 |
| | | x57559; m55698; m60182; af213351 | |
| PIV-3 | Fusion | CCRI-15672 | 45 |
| | | CCRI-15867 | 46 |
| | | D00016; M14892; M21649; S82195; | |
| | | S82195i; X05303; z11575 | |
| | | CCRI-15706 | 47 |
| Coronavirus OC43 | matrix | ay391777; ay585228; ay585229; m93390; | |
| | | ay391777; ay585228; ay585229; | |
| | | ay391777; ay903459; ay382775; ay903460 | |
| Coronavirus 229E | Polymerase | x69721; af304460; nc_002645 | |
| Coronavirus SARS- | Polymerase | zj01_ay297028;; -hku-39849_ay278491; | |
| CoV | | sin2748_ay283797; urbani_ay278741; | |
| | | tor2_ay274119_v3; tw1_ay291451; | |
| | | sin2774_ay283798; sin2748_ay283797; | |
| | | sin2679_ay283796; sin2677_ay283795; | |
| | | sin2500_ay283794;; gz01_ay278489; | |
| | | cuhk-w1_ay278554; cuhk-su10_ay282752; | |
| | | bj02_ay278487; bj01_ay278488; - | |
| | | taiwantc3_ay348314; taiwantc2_ay338175; | |
| | | twc3_ay362699; sod_ay461660; | |
| | | pumc03_ay357076; pumc02_ay357075; | |
| | | gz50_ay304495; cuhk-ag03_ay345988; - | |
| | | cuhk-ag02_ay345987; -cuhk- | |
| | | ag01_ay345986; ay427439; - | |
| | | twc2_ay362698; -tw9_ay502932; - | |
| | | tw8_ay502931; -tw7_ay502930; - | |
| | | tw6_ay502929; -tw5_ay502928; - | |
| | | tw10_ay502923; -sino3-11_ay485278; - | |
| | | fra_ay310120; tw11_ay502924; | |
| | | shanghaiqxc1_ay463059; -sino1- | |
| | | 11_ay485277; bj03_ay278490; - | |
| | | gd69_ay313906; -whu_ay394850; - | |
| | | twy_ap006561; -tws_ap006560; - | |
| | | twk_ap006559; -twj_ap006558; - | |
| | | twh_ap006557; -twc_ay321118; - | |
| | | hsr1_ay323977; -frankfurt_ay291315;; - | |
| | | zmy1_ay351680; -taiwantc1_ay338174; | |
| | | bj04_ay279354; -shanghaiqxc2_ay463060; | |
| | | bat_dq084200i; bat_dq084199i; | |
| | | bat_dq022305i; ay394981; ay394982; | |
| | | ay394983; ay394986; ay394987; | |
| | | ay394988; ay545914; | |
| | | ay545915; ay545916; ay545917; | |
| | | ay545918; ay545919; ay559090; | |
| | | ay559095; ay559096; ay568539; | |
| | | ay595412; ay613949; ay613950; | |
| | | ay654624; ay686863; ay686864; | |
| | | ay714217; ay772062; nc_004718; | |
| | | ay304486; ay304488; ay350750; | |
| | | ay394989; ay394990; ay394991; | |
| | | ay394992; ay394993; ay559086; | |
| | | ay559087; ay559088; ay559089; | |
| | | ay559091; ay559092; ay559093; | |
| | | ay559094; ay559097; ay313906 | |
| Coronavirus NL | Polymerase | CCRI-16014 | 51 |
| | | dq445911; ay518894; nc_005831; | |
| | | dq445912 | |
| Adenovirus | hexon | CCRI-15636 | 53 |
| | | CCRI-15625 | 54 |
| | | CCRI-15492 | 55 |
| | | CCRI-15517 | 56 |
| | | CCRI-15673 | 58 |
| | | type14_ay803294; type14_dq149612; | |
| | | type_21-ay008279; type_21-ay380333; | |
| | | type_7-af515814; type_7-ay337253; | |
| | | type_7-ay337254; type_7-ay337255; | |
| | | type_7-ay337256; type_7-ay337257; | |
| | | type_7-ay337258; type_7-ay380327; | |
| | | type_7-ay628149; type_7af065067; | |
| | | type_7a-55142-af065067; type_7a- | |
| | | af065067; type_1-af161559; type_2- | |
| | | af542118; type_2-af542120; type_2- | |
| | | aj278924; type_2-aj293901; type_2- | |
| | | aj293902; type_2-aj293903; type_2- | |
| | | aj293904; type_2-aj293905; type_2- | |
| | | ay224391; type_2-ay224392; type_2- | |
| | | ay375454; type_2-ay628143; type_2- | |
| | | ay628144; type_2-ay628145; type_5- | |
| | | af542109; type_5-af542116; type_5- | |
| | | af542119; type_5-af542121; type_5- | |
| | | af542124; type_5-af542128; type_5- | |
| | | af542130; type_5-ay628141; type_5- | |
| | | ay628141i; type_5-ay628142; type4- | |
| | | af161569; type_4-af065063; type_4- | |
| | | af065064; type_4-af542122; type_4- | |
| | | ay337237; type_4-ay337238; type_4- | |
| | | ay337239; type_4-ay337240; type_4- | |
| | | ay337241; type_4-ay337242; type_4- | |
| | | ay337243; type_4-ay337244; type_4- | |
| | | ay337245; type_4-ay337246; type_4- | |
| | | ay337247; type_4-ay337248; type_4- | |
| | | ay337249; type_4-ay337250; type_4- | |
| | | ay337251; type_4-ay337252; type_4- | |
| | | ay380321; type_4-ri-67-af065062; type_4- | |
| | | x84646 | |
| Influenza B | Matrix | CCRI-15829 | 59 |
| | | CCRI-15750 | 60 |
| | | CCRI-15803 | 61 |
| | | CCRI-15751 | 62 |
| | | CCRI-15804 | 63 |
| | | CCRI-15593 | 64 |
| | | CCRI-15837 | 65 |
| | | CCRI-15831 | 66 |
| | | ab059256; ab059248; m20176; | |
| | | m20175; m14909; j02094; | |
| | | ay260955; ay260941; ay044171; | |
| | | af100392; af100391; af100390; | |
| | | af100389; | |
| | | af100388; af100387; af100386; | |
| | | af100385; af100384; af100383; | |
| | | af100382; af100381; af100380; | |
| | | af100379; af100378; af100377; | |
| | | af100376; af100375; af100374; | |
| | | af077348; ab120275; ab120274; | |
| | | ab120273; ab120272; ab120271; | |
| | | ab120270; ab120269; ab120268; | |
| | | ab120267; ab120266; ab120265; | |
| | | ab120264; ab120263; ab120262; | |
| | | ab120261; ab120260; ab120259; | |
| | | ab120258; ab036879; ab036878; | |
| | | ab036877; ay504621; ay504613; | |
| | | aj783379; aj783378; | |
| | | aj783386; aj783389; aj783388; | |
| | | aj783393; aj783391; ay581983; | |
| | | ay581982; ay581981; ay581980; | |
| | | ay504605; ay581979; aj783383; | |
| | | aj783380; aj783392; aj783382; | |
| | | aj783376; aj783381; aj783385; | |
| | | aj783384; aj783395; aj783394; | |
| | | aj783390; aj783387 | |
| PIV-4 | Fusion | CCRI-15568 | 68 |
| | | CCRI-15512 | 69 |
| | | CCRI-15622 | 70 |
| | | CCRI-15530 | 71 |
| | | CCRI-15836 | 72 |
| | | CCRI-15774 | 73 |
| | | CCRI-15546 | 74 |
| | | CCRI-15697 | 75 |
| | | CCRI-15701 | 76 |
| | | d49822; ay286479; d49821 | |

*The gene sequences determined through this study are identified with a CCRI (Collection of “Centre de Recherche en Infectiologie”) number corresponding to the viral isolate and a SEQ ID NO was assigned for each of them. Gene sequences available in public databases are identified with their Genbank accession number and no SEQ ID NO was assigned to any of these sequences.

TABLE 2
Multiplex RT-PCR assays for detection of respiratory viruses

| | | | Primers | | | Sensitivity |
|---|---|---|---|---|---|---|
| | | | SEQ ID | Probes | Amplicon | (genome |
| Multiplex | Virus | Gene | NO. | SEQ ID NO. | length | copies) |
| 1 | Rhinovirus/Enterovirus | 5′ non-coding | 78, 79 | 106, 107, 108, | 119 bp | 100 copies |
| | | | | 109, 110, 111, | | |
| | | | | 112, 113, 114, | | |
| | | | | 115, 116, 117, | | |
| | | | | 118, 119, 120, | | |
| | | | | 194, 195, 196 | | |
| | Human respiratory | Nucleocapsid | 80, 81 | 121, 122, 123, | 109 bp | 50 copies |
| | syncytial Virus | | | 124, 125, 126, | | |
| | | | | 127, 197, 198, | | |
| | | | | 199, 200 | | |
| | Human | Nucleocapsid | 82, 83 | 128, 129, 130, | 105 bp | 25 copies |
| | metapneumovirus | | | 131, 132, 133 | | |
| 2 | Influenza A | matrix | 84, 85 | 134, 135, 136, | 120 bp | 100 copies |
| | | | | 137, 138, 139, | | |
| | | | | 140, 141, 142, | | |
| | | | | 143, 144, 145, | | |
| | | | | 146, 147, 148, | | |
| | | | | 149, 150, 151, | | |
| | | | | 152, 201, 202, | | |
| | | | | 203, 204, 205, | | |
| | | | | 206, 207, 208 | | |
| | Parainfluenza type 1 | Fusion | 86, 87 | 153, 154 | 153 bp | 50 copies |
| | Parainfluenza type 2 | Fusion | 88, 89 | 155, 156, 157 | 123 bp | 50 copies |
| | Parainfluenza type 3 | Fusion | 90, 91 | 158, 209, 225, | 162 bp | 10 copies |
| | | | | 226, 227, 228, | | |
| | | | | 229, | | |
| 3 | Coronavirus SARS-CoV | Polymerase | 92, 93 | 159, 160 | 201 bp | 10 copies |
| | Coronavirus | Polymerase | 94, 95 | 161 | 205 bp | 100 copies |
| | 229E | | | | | |
| | Coronavirus NL | Polymerase | 94, 95 | 162 | 205 bp | 100 copies |
| | Coronavirus | Matrix | 96, 97 | 163, 164 | 179 bp | 25 copies |
| | OC43 | | | | | |
| 4 | Adenovirus | Hexon | 98, 99 | 165, 166, 167, | 243 bp | 10 copies |
| | | | | 168, 169, 170, | | |
| | | | | 171, 172, 173, | | |
| | | | | 174, 175, 176, | | |
| | | | | 177, 178 | | |
| | Influenza B | Matrix | 100, | 179, 180, 181, | 141 bp | 25 copies |
| | | | 101 | 182, 183, 210, | | |
| | | | | 211, 212, 213, | | |
| | | | | 214 | | |
| | Parainfluenza type 4 | Fusion | 102, | 184, 185, 186, | 118 bp | 10 copies |
| | | | 103 | 187, 188 | | |

TABLE 3
Percentage of primers and probes which were
retained for the present invention.

| | | | | % primers | % probes |
|---|---|---|---|---|---|
| Multiplex | Virus | Primers | Probes | selected | selected |
| 1 | Enterovirus | 2/6 | 15/15 | 33.3 | 100 |
| | RSV | 2/5 | 7/11 | 40 | 64 |
| | hMPV | 2/6 | 6/6 | 33.3 | 100 |
| 2 | Influenza A | 2/12 | 19/19 | 16.7 | 100 |
| | PIV-1 | 2/13 | 2/5 | 15.4 | 40 |
| | PIV-2 | 2/7 | 3/6 | 28.6 | 50 |
| | PIV-3 | 2/7 | 1/5 | 28.6 | 20 |
| 3 | SARS-CoV | 2/10 | 1/4 | 20 | 25 |
| | 229E | 2/19 | 1/4 | 10.5 | 25 |
| | NL | 2/19 | 1/4 | 10.5 | 25 |
| | OC43 | 2/9 | 1/4 | 22.2 | 25 |
| 4 | Adenovirus | 2/23 | 7/31 | 8.7 | 21.8 |
| | Influenza B | 2/11 | 5/17 | 18.1 | 29.4 |
| | PIV-4 | 2/11 | 5/13 | 18.1 | 38.4 |

TABLE 4
Primers and probes used to amplify the internal control
template for each optimized multiplex RT-PCR assay.

| Internal Control* | Primers (SEQ ID NO) | Probes (SEQ ID NO) |
|---|---|---|
| Internal Control | 80, 83 | 192, 223, 224 |
| multiplex 1 | | |
| Internal Control | 87, 88 | 189, 216, 217 |
| multiplex 2 | | |
| Internal Control | 92, 95 | 191, 215, 221, 222 |
| multiplex 3 | | |
| Internal Control | 98, 103 | 190, 218, 219, 220 |
| multiplex 4 | | |
| Cell Lysis Control | 104, 105 | 193 |

*The internal control template, which is present in each PCR reaction, allows to verify the efficiency of each PCR amplification and/or microarray hybridization as well as to ensure that there is no significant inhibition of the nucleic acid amplification and/or detection processes. The cell lysis control consists of cells added to each PCR reaction to also verify the efficiency of the cell lysis and nucleic acid extraction processes.

TABLE 5
Cloned RT-PCR products serving as internal control template and
used for determination of the analytical sensitivity of the assays.

| | | | SEQ |
|---|---|---|---|
| Respiratory Virus | Target Gene | Clone | ID NO |
| Influenza A | Matrix | clone Influenza A 15590 | 15 |
| RSV | Nucleocapsid | clone RSV 15552 | 21 |
| hMPV | Nucleocapsid | clone hMPV 15597 | 22 |
| Enterovirus | 5′non-coding | clone entero 15531 | 37 |
| PIV-1 | Fusion | clone PIV-1 15598 | 41 |
| PIV-2 | Fusion | clone PIV-2 15641 | 44 |
| PIV-3 | Fusion | clone PIV-3 15706 | 48 |
| Coronavirus OC43 | Matrix | clone OC43 matrix | 49 |
| Coronavirus 229E | Polymerase | clone nsp9-229E | 50 |
| Coronavirus SARS- | Polymerase | clone SARS-CoV nsp9 | 52 |
| CoV | | | |
| Coronavirus NL | Polymerase | clone NL polymerase | 51 |
| | | 16014 | |
| Adenovirus | Hexon | clone Adenovirus 15517 | 57 |
| Influenza B | Matrix | clone Influenza B 15593 | 67 |
| PIV-4 | Fusion | clone PIV-4 15836 | 77 |

1.-72. (canceled)

73. A method of detecting a respiratory virus, the method comprising exposing a sample containing or suspected of containing a pathogen with oligonucleotide mixtures comprising multiple oligonucleotide species capable of specific binding with a genetic material of a respiratory virus species, said multiple oligonucleotide species comprising multiple sets of primer pairs selected from the group consisting of:
a) a nucleic acid comprising from 0 to 5 nucleotide addition or deletion at a 5′ end of SEQ ID NO.:78 and a nucleic acid comprising from 0 to 5 nucleotide addition or deletion at a 5′ end of SEQ ID NO.:79;
b) a nucleic acid comprising from 0 to 5 nucleotide addition or deletion at a 5′ end of SEQ ID NO.:80 and a nucleic acid comprising from 0 to 5 nucleotide addition or deletion at a 5′ end of SEQ ID NO.:81;
c) a nucleic acid comprising from 0 to 5 nucleotides addition or deletion at a 5′ end of SEQ ID NO.:82 and a nucleic acid comprising from 0 to 5 nucleotide addition or deletion at a 5′ end of SEQ ID NO.:83;
d) a nucleic acid comprising from 0 to 5 nucleotide addition or deletion at a 5′ end of SEQ ID NO.: 84 and a nucleic acid comprising from 0 to 5 nucleotide addition or deletion at a 5′ end of SEQ ID NO.:85;
e) a nucleic acid comprising from 0 to 5 nucleotide addition or deletion at a 5′ end of SEQ ID NO.:86 and a nucleic acid comprising from 0 to 5 nucleotide addition or deletion at a 5′ end of SEQ ID NO.:87;
f) a nucleic acid comprising from 0 to 5 nucleotide addition or deletion at a 5′ end of SEQ ID NO.:88 and a nucleic acid comprising from 0 to 5 nucleotide addition or deletion at a 5′ end of SEQ ID NO.:89;
g) a nucleic acid comprising from 0 to 5 nucleotide addition or deletion at a 5′ end of SEQ ID NO.:90 and a nucleic acid comprising from 0 to 5 nucleotide addition or deletion at a 5′ end of SEQ ID NO.:91;
h) a nucleic acid comprising from 0 to 5 nucleotide addition or deletion at a 5′ end of SEQ ID NO.:92 and a nucleic acid comprising from 0 to 5 nucleotide addition or deletion at a 5′ end of SEQ ID NO.:93;
i) a nucleic acid comprising from 0 to 5 nucleotide addition or deletion at a 5′ end of SEQ ID NO.:94 and a nucleic acid comprising from 0 to 5 nucleotide addition or deletion at a 5′ end of SEQ ID NO.:95;
j) a nucleic acid comprising from 0 to 5 nucleotide addition or deletion at a 5′ end of SEQ ID NO.:96 and a nucleic acid comprising from 0 to 5 nucleotide addition or deletion at a 5′ end of SEQ ID NO.:97;
k) a nucleic acid comprising from 0 to 5 nucleotide addition or deletion at a 5′ end of SEQ ID NO.:98 and a nucleic acid comprising from 0 to 5 nucleotide addition or deletion at a 5′ end of SEQ ID NO.:99;
l) a nucleic acid comprising from 0 to 5 nucleotide addition or deletion at a 5′ end of SEQ ID NO.:100 and a nucleic acid comprising from 0 to 5 nucleotide addition or deletion at a 5′ end of SEQ ID NO.:101;
m) a nucleic acid comprising from 0 to 5 nucleotide addition or deletion at a 5′ end of SEQ ID NO.:102 and a nucleic acid comprising from 0 to 5 nucleotide addition or deletion at a 5′ end of SEQ ID NO.:103; and
n) combination of any one of a) to m).

74. The method of claim 73, wherein:
A) human enteroviruses, rhinoviruses, human respiratory syncitial viruses and human metapneumoviruses are amplified by the set of primer pair defined in a), b) and c);
B) influenza A viruses, parainfluenza type 1 viruses, parainfluenza type 2 viruses and parainfluenza type 3 viruses are amplified by the set of primer pair defined in d), e), f) and g);
C) coronaviruses SARS-CoV, 229E/NL and OC-43 are amplified by the set of primer pair defined in h), i) and j);
D) adenoviruses, influenza B viruses and parainfluenza type 4 viruses are amplified by the set of primer pair defined in k), l) and m).

75. The method of claim 73, wherein the multiple oligonucleotides species comprise probes selected from the group consisting of a nucleic acid comprising from 0 to 5 nucleotide addition, deletion or combination of addition and deletion at a 5′ end and/or 3′ end thereof any one of SEQ ID NO.:106, SEQ ID NO.:107, SEQ ID NO.:108, SEQ ID NO.:109, SEQ ID NO.:110, SEQ ID NO.:111, SEQ ID NO.:112, SEQ ID NO.:113, SEQ ID NO.:114, SEQ ID NO.:115, SEQ ID NO.:116, SEQ ID NO.:117, SEQ ID NO.:118, SEQ ID NO.:119, SEQ ID NO.:120, SEQ ID NO.:121, SEQ ID NO.:122, SEQ ID NO.:123, SEQ ID NO.:124, SEQ ID NO.:125, SEQ ID NO.:126, SEQ ID NO.:127, SEQ ID NO.:128, SEQ ID NO.:129, SEQ ID NO.:130, SEQ ID NO.:131, SEQ ID NO.:132, SEQ ID NO.:133, SEQ ID NO.:134, SEQ ID NO.:135, SEQ ID NO.:136, SEQ ID NO.:137, SEQ ID NO.:138, SEQ ID NO.:139, SEQ ID NO.:140, SEQ ID NO.:141, SEQ ID NO.:142, SEQ ID NO.:143, SEQ ID NO.144, SEQ ID NO.:145, SEQ ID NO.:146, SEQ ID NO.:147, SEQ ID NO.:148, SEQ ID NO.:149, SEQ ID NO.:150, SEQ ID NO.:151, SEQ ID NO.:152, SEQ ID NO.:153, SEQ ID NO.:154, SEQ ID NO.:155, SEQ ID NO.:156, SEQ ID NO.:157, SEQ ID NO.:158, SEQ ID NO.:159, SEQ ID NO.:160, SEQ ID NO.:161, SEQ ID NO.:162, SEQ ID NO.:163, SEQ ID NO.:164, SEQ ID NO.:165, SEQ ID NO.:166, SEQ ID NO.:167, SEQ ID NO.:168, SEQ ID NO.:169, SEQ ID NO.:170, SEQ ID NO.:171, SEQ ID NO.:172, SEQ ID NO.:173, SEQ ID NO.:174, SEQ ID NO.:175, SEQ ID NO.:176, SEQ ID NO.:177, SEQ ID NO.:178, SEQ ID NO.:179, SEQ ID NO.:180, SEQ ID NO.:181, SEQ ID NO.:182, SEQ ID NO.:183, SEQ ID NO.:184, SEQ ID NO.:185, SEQ ID NO.:186, SEQ ID NO.:187, SEQ ID NO.:188, SEQ ID NO.:194, SEQ ID NO.:195, SEQ ID NO.:196, SEQ ID NO.:197, SEQ ID NO.:198, SEQ ID NO.:199, SEQ ID NO.:200, SEQ ID NO.:201, SEQ ID NO.:202, SEQ ID NO.:203, SEQ ID NO.:204, SEQ ID NO.:205, SEQ ID NO.:206, SEQ ID NO.:207, SEQ ID NO.:208, SEQ ID NO.:209, SEQ ID NO.:210, SEQ ID NO.:211, SEQ ID NO.:212, SEQ ID NO.:213, SEQ ID NO.:214, SEQ ID NO.:225, SEQ ID NO.:226, SEQ ID NO.:227, SEQ ID NO.:228, SEQ ID NO.:229, and the complement and combinations thereof.

76. A mixture of oligonucleotides comprising at least two oligonucleotides selected from the group consisting of:
a) an oligonucleotide comprising or consisting of the sequence selected from the group consisting of SEQ ID NO.:78, SEQ ID NO.:79, SEQ ID NO.:80, SEQ ID NO.:81, SEQ ID NO.:82, SEQ ID NO.:83, SEQ ID NO.:84, SEQ ID NO.:85, SEQ ID NO.:86, SEQ ID NO.:87, SEQ ID NO.:88, SEQ ID NO.:89, SEQ ID NO.:90, SEQ ID NO.:91, SEQ ID NO.:92, SEQ ID NO.:93, SEQ ID NO.:94, SEQ ID NO.:95, SEQ ID NO.:96, SEQ ID NO.:97, SEQ ID NO.:98, SEQ ID NO.:99, SEQ ID NO.:100, SEQ ID NO.:101, SEQ ID NO.:102 and SEQ ID NO.:103;
b) the oligonucleotide of a) comprising from 0 to 5 additional nucleotides at a 5′ end thereof;
c) the oligonucleotide of a) comprising from 0 to 5 nucleotides deletion at a 5′ end thereof; and
d) a complement of any one of a) to c).

77. The mixture of claim 76, comprising an oligonucleotide pair selected from the group consisting of:
a) a nucleic acid comprising from 0 to 5 nucleotide addition or deletion at a 5′ end of SEQ ID NO.:78 and a nucleic acid comprising from 0 to 5 nucleotide addition or deletion at a 5′ end of SEQ ID NO.:79;
b) a nucleic acid comprising from 0 to 5 nucleotide addition or deletion at a 5′ end of SEQ ID NO.:80 and a nucleic acid comprising from 0 to 5 nucleotide addition or deletion at a 5′ end of SEQ ID NO.:81;
c) a nucleic acid comprising from 0 to 5 nucleotides addition or deletion at a 5′ end of SEQ ID NO.:82 and a nucleic acid comprising from 0 to 5 nucleotide addition or deletion at a 5′ end of SEQ ID NO.:83;
d) a nucleic acid comprising from 0 to 5 nucleotide addition or deletion at a 5′ end of SEQ ID NO.:84 and a nucleic acid comprising from 0 to 5 nucleotide addition or deletion at a 5′ end of SEQ ID NO.:85;
e) a nucleic acid comprising from 0 to 5 nucleotide addition or deletion at a 5′ end of SEQ ID NO.:86 and a nucleic acid comprising from 0 to 5 nucleotide addition or deletion at a 5′ end of SEQ ID NO.:87;
f) a nucleic acid comprising from 0 to 5 nucleotide addition or deletion at a 5′ end of SEQ ID NO.:88 and a nucleic acid comprising from 0 to 5 nucleotide addition or deletion at a 5′ end of SEQ ID NO.:89;
g) a nucleic acid comprising from 0 to 5 nucleotide addition or deletion at a 5′ end of SEQ ID NO.:90 and a nucleic acid comprising from 0 to 5 nucleotide addition or deletion at a 5′ end of SEQ ID NO.:91;
h) a nucleic acid comprising from 0 to 5 nucleotide addition or deletion at a 5′ end of SEQ ID NO.:92 and a nucleic acid comprising from 0 to 5 nucleotide addition or deletion at a 5′ end of SEQ ID NO.:93;
i) a nucleic acid comprising from 0 to 5 nucleotide addition or deletion at a 5′ end of SEQ ID NO.:94 and a nucleic acid comprising from 0 to 5 nucleotide addition or deletion at a 5′ end of SEQ ID NO.:95;
j) a nucleic acid comprising from 0 to 5 nucleotide addition or deletion at a 5′ end of SEQ ID NO.:96 and a nucleic acid comprising from 0 to 5 nucleotide addition or deletion at a 5′ end of SEQ ID NO.:97;
k) a nucleic acid comprising from 0 to 5 nucleotide addition or deletion at a 5′ end of SEQ ID NO.:98 and a nucleic acid comprising from 0 to 5 nucleotide addition or deletion at a 5′ end of SEQ ID NO.:99;
l) a nucleic acid comprising from 0 to 5 nucleotide addition or deletion at a 5′ end of SEQ ID NO.:100 and a nucleic acid comprising from 0 to 5 nucleotide addition or deletion at a 5′ end of SEQ ID NO.:101;
m) a nucleic acid comprising from 0 to 5 nucleotide addition or deletion at a 5′ end of SEQ ID NO.:102 and a nucleic acid comprising from 0 to 5 nucleotide addition or deletion at a 5′ end of SEQ ID NO.:103 and
n) combination of any one of a) to m) thereof.

78. The mixture of claim 76, wherein said mixture is for the specific detection or amplification of one or more viruses, wherein:
A) for the specific detection or amplification of a human enterovirus, a rhinovirus, a human respiratory syncytial virus and a human metapneumovirus, the mixture comprises at least two oligonucleotides selected from the group consisting of:
a) an oligonucleotide comprising or consisting of the sequence selected from the group consisting of SEQ ID NO.:78, SEQ ID NO.:79, SEQ ID NO.:80, SEQ ID NO.:81, SEQ ID NO.:82, SEQ ID NO.:83;
b) the oligonucleotide of a) comprising from 0 to 5 additional nucleotides at a 5′ end thereof;
c) the oligonucleotide of a) comprising from 0 to 5 nucleotides deletion at a 5′ end thereof; and
d) a complement of any one of a), b) or c);
B) for the specific detection or amplification of an influenza A virus, a parainfluenza type 1 virus, a parainfluenza type 2 virus and a parainfluenza type 3 virus, the mixture comprises at least two oligonucleotides selected from the group consisting of:
e) an oligonucleotide comprising or consisting of the sequence selected from the group consisting of SEQ ID NO.:84, SEQ ID NO.:85, SEQ ID NO.:86, SEQ ID NO.:87, SEQ ID NO.:88, SEQ ID NO.:89, SEQ ID NO.:90, SEQ ID NO.:91;
f) the oligonucleotide of a) comprising from 0 to 5 additional nucleotides at a 5′ end thereof,
g) the oligonucleotide of a) comprising from 0 to 5 nucleotides deletion at a 5′ end thereof; and
h) a complement of any one of e), f) or g;
C) for the specific detection or amplification of a coronavirus SARS-CoV, a coronavirus 229E, a coronavirus NL and a coronavirus OC43, the mixture comprises at least two oligonucleotides selected from the group consisting of:
i) an oligonucleotide comprising or consisting of the sequence selected from the group consisting of SEQ ID NO.:92, SEQ ID NO.:93, SEQ ID NO.:94, SEQ ID NO.:95, SEQ ID NO.:96, SEQ ID NO.:97;
j) the oligonucleotide of a) comprising from 0 to 5 additional nucleotides at a 5′ end thereof;
k) the oligonucleotide of a) comprising from 0 to 5 nucleotides deletion at a 5′ end thereof; and
l) a complement of any one of i), j) or k);
D) for the specific detection or amplification of an adenovirus, an influenza B virus and a parainfluenza type 4 virus, the mixture comprises at least two oligonucleotides selected from the group consisting of:
m) an oligonucleotide comprising or consisting of the sequence selected from the group consisting of SEQ ID NO.:98, SEQ ID NO.:99, SEQ ID NO.:100, SEQ ID NO.:101, SEQ ID NO.:102 and SEQ ID NO.:103;
n) the oligonucleotide of a) comprising from 0 to 5 additional nucleotides at a 5′ end thereof;
o) the oligonucleotide of a) comprising from 0 to 5 nucleotides deletion at a 5′ end thereof; and
p) a complement of any one of m), n) or o).

79. An oligonucleotide selected from the group consisting of:
a) an oligonucleotide having or consisting of the sequence selected from the group consisting of SEQ ID NO.:106 to SEQ ID NO.:188, SEQ ID NO.:194 to SEQ ID NO.:213, SEQ ID NO.:214 and SEQ ID NO.:225 to SEQ ID NO.:229;
b) the nucleic acid of a) comprising from 0 to 5 additional nucleotides at a 5′ end and/or 3′ end thereof;
c) the nucleic acid of a) comprising from 0 to 5 nucleotides deletion at a 5′ end and/or 3′ end thereof;
d) a nucleic acid of a) comprising from 0 to 5 additional nucleotides at one of a 5′ end or 3′ end and a deletion of from 0 to 5 nucleotides at the other of a 5′ end or 3′ end thereof; and
e) a complement of any one a) to d).

80. The oligonucleotide of claim 79, wherein said oligonucleotide is capable of binding to a human respiratory syncytial virus and wherein said oligonucleotide is selected from the group consisting of:
a) an oligonucleotide having or consisting of the sequence selected from the group consisting of SEQ ID NO.:121, SEQ ID NO.:122, SEQ ID NO.:123, SEQ ID NO.:124, SEQ ID NO.:125, SEQ ID NO.:126, SEQ ID NO.:127, SEQ ID NO.:197, SEQ ID NO.:198, SEQ ID NO.:199 and SEQ ID NO.:200;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

81. The oligonucleotide of claim 79, wherein said oligonucleotide is capable of binding to a human metapneumovirus and wherein said oligonucleotide is selected from the group consisting of:
a) an oligonucleotide having or consisting of the sequence selected from the group consisting of SEQ ID NO.:128, SEQ ID NO.:129, SEQ ID NO.:130, SEQ ID NO.:131, SEQ ID NO.:132 and SEQ ID NO.:133;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

82. The oligonucleotide of claim 79, wherein said oligonucleotide is capable of binding to an influenza A virus and wherein said oligonucleotide is selected from the group consisting of:
a) an oligonucleotide having or consisting of the sequence selected from the group consisting of SEQ ID NO.:134, SEQ ID NO.:135, SEQ ID NO.:136, SEQ ID NO.:137, SEQ ID NO.:138, SEQ ID NO.:139, SEQ ID NO.:140, SEQ ID NO.:141, SEQ ID NO.:142, SEQ ID NO.:143, SEQ ID NO.144, SEQ ID NO.:145, SEQ ID NO.:146, SEQ ID NO.:147, SEQ ID NO.:148, SEQ ID NO.:149, SEQ ID NO.:150, SEQ ID NO.:151, SEQ ID NO.:152, SEQ ID NO.:201, SEQ ID NO.:202, SEQ ID NO.:203, SEQ ID NO.:204, SEQ ID NO.:205, SEQ ID NO.:206, SEQ ID NO.:207 and SEQ ID NO.:208;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

83. The oligonucleotide of claim 79, wherein said oligonucleotide is capable of binding to a parainfluenza type 1 virus and wherein said oligonucleotide is selected from the group consisting of:
a) an oligonucleotide having or consisting of the sequence selected from the group consisting of SEQ ID NO.:153 and SEQ ID NO.:154;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

84. The oligonucleotide of claim 79, wherein said oligonucleotide is capable of binding to a parainfluenza type 2 virus and wherein said oligonucleotide is selected from the group consisting of:
a) an oligonucleotide having or consisting of the sequence selected from the group consisting of SEQ ID NO.:155, SEQ ID NO.:156 and SEQ ID NO.:157;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

85. The oligonucleotide of claim 79, wherein said oligonucleotide is capable of binding to a parainfluenza type 3 virus and wherein said oligonucleotide is selected from the group consisting of:
a) an oligonucleotide having or consisting of the sequence selected from the group consisting of SEQ ID NO.:158, SEQ ID NO.:209 and SEQ ID NOs.:225 to:229,
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

86. The oligonucleotide of claim 79, wherein said oligonucleotide is capable of binding to a coronavirus 229E wherein said oligonucleotide is selected from the group consisting of:
a) an oligonucleotide having or consisting of the sequence selected from the group consisting of SEQ ID NO.:161;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

87. The oligonucleotide of claim 79, wherein said oligonucleotide is capable of binding to a coronavirus NL wherein said oligonucleotide is selected from the group consisting of:
a) an oligonucleotide having or consisting of the sequence selected from the group consisting of SEQ ID NO.:162;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

88. The oligonucleotide of claim 79, wherein said oligonucleotide is capable of binding to a coronavirus 0C43 wherein said oligonucleotide is selected from the group consisting of:
a) an oligonucleotide having or consisting of the sequence selected from the group consisting of SEQ ID NO.:163 and SEQ ID NO.:164;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

89. The oligonucleotide of claim 79, wherein said oligonucleotide is capable of binding to an adenovirus wherein said oligonucleotide is selected from the group consisting of:
a) an oligonucleotide having or consisting of the sequence selected from the group consisting of SEQ ID NO.:165, SEQ ID NO.:166, SEQ ID NO.:167, SEQ ID NO.:168, SEQ ID NO.:169, SEQ ID NO.:170, SEQ ID NO.:171, SEQ ID NO.:172, SEQ ID NO.:173, SEQ ID NO.:174, SEQ ID NO.:175, SEQ ID NO.:176, SEQ ID NO.:177 and SEQ ID NO.:178;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

90. The oligonucleotide of claim 79, wherein said oligonucleotide is capable of binding to an influenza B virus wherein said oligonucleotide is selected from the group consisting of:
a) an oligonucleotide having or consisting of the sequence selected from the group consisting of SEQ ID NO.:179, SEQ ID NO.:180, SEQ ID NO.:181, SEQ ID NO.:182, SEQ ID NO.:183, SEQ ID NO.:210, SEQ ID NO.:211, SEQ ID NO.:212, SEQ ID NO.:213 and SEQ ID NO.:214;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

91. The oligonucleotide of claim 79, wherein said oligonucleotide is capable of binding to a parainfluenza 4 virus wherein said oligonucleotide is selected from the group consisting of:
a) an oligonucleotide having or consisting of the sequence selected from the group consisting of SEQ ID NO.:184, SEQ ID NO.:185, SEQ ID NO.:186, SEQ ID NO.:187 and SEQ ID NO.:188;
b) the oligonucleotide of a) wherein the oligonucleotide comprises from 0 to 5 additional nucleotides at a 5′ end and/or 3′ end thereof;
c) the oligonucleotide of a) wherein the oligonucleotide comprises a deletion of from 0 to 5 nucleotides at a 5′ end and/or 3′ end thereof;
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof; and
e) a complement of any one of the above.

92. A kit comprising a plurality of oligonucleotides of from 10 to 50 nucleotides long capable of specific binding to a respiratory virus gene selected from the group consisting of (i) a matrix gene of the influenza A virus (ii) a matrix gene of the influenza B virus, (iii) a nucleocapsid gene of the human respiratory syncytial virus, (iv) a nucleocapsid gene of the human metapneumovirus, (v) a 5′-non-coding region of the human enterovirus, (vi) a 5′-non-coding region of the rhinovirus, (vii) a fusion gene of the parainfluenza virus type 1, (viii) a fusion gene of the parainfluenza virus type 2, (ix) a fusion gene of the parainfluenza virus type 3, (x) a fusion gene of the parainfluenza virus type 4, (xi) a matrix gene of the coronavirus OC43, (xii) a polymerase gene of the coronavirus NL, (xiii) a polymerase gene of the coronavirus 229E, (xiv) a polymerase gene of the coronavirus SARS-CoV and (xv) a hexon region of the adenovirus, wherein the plurality of oligonucleotides are capable of amplifying the gene under similar amplification conditions or are capable of hybridizing to the gene under similar hybridization conditions.

93. The kit of claim 92, wherein said plurality of oligonucleotides is selected from the group consisting of:
a) an oligonucleotide having or consisting of the sequence selected from the group consisting of SEQ ID NO.:106 to SEQ ID NO.:188, SEQ ID NO.:194 to SEQ ID NO.:213, SEQ ID NO.:214 and SEQ ID NO.:225 to SEQ ID NO.:229;
b) the nucleic acid of a) comprising from 0 to 5 additional nucleotides at a 5′ end and/or 3′ end thereof;
c) the nucleic acid of a) comprising from 0 to 5 nucleotides deletion at a 5′ end and/or 3′ end thereof;
d) a nucleic acid of a) comprising from 0 to 5 additional nucleotides at one of a 5′ end or 3′ end and a deletion of from 0 to 5 nucleotides at the other of a 5′ end or 3′ end thereof; and
e) a complement of any one a) to d).

94. An array comprising:
a) at least one member selected from the group consisting of an oligonucleotide comprising from 0 to 5 nucleotide addition and/or deletion to SEQ ID NO.:106, SEQ ID NO.:107, SEQ ID NO.:108, SEQ ID NO.:109, SEQ ID NO.:110, SEQ ID NO.:111, SEQ ID NO.:112, SEQ ID NO.:113, SEQ ID NO.:114, SEQ ID NO.:115, SEQ ID NO.:116, SEQ ID NO.:117, SEQ ID NO.:118, SEQ ID NO.:119, SEQ ID NO.:120, SEQ ID NO.:194, SEQ ID NO.:195 and SEQ ID NO.:196 or to a complement thereof and wherein the addition and/or deletion is located at a 5′ end and/or 3′ end of the nucleic acid sequence;
b) at least one member selected from the group consisting of an oligonucleotide comprising from 0 to 5 nucleotide addition and/or deletion to SEQ ID NO.:121, SEQ ID NO.:122, SEQ ID NO.:123, SEQ ID NO.:124, SEQ ID NO.:125, SEQ ID NO.:126, SEQ ID NO.:127, SEQ ID NO.:197, SEQ ID NO.:198, SEQ ID NO.:199 and SEQ ID NO.:200 or to a complement thereof and wherein the addition and/or deletion is located at a 5′ end and/or 3′ end of the nucleic acid sequence;
c) at least one member selected from the group consisting of an oligonucleotide comprising from 0 to 5 nucleotide addition and/or deletion to SEQ ID NO.:128, SEQ ID NO.:129, SEQ ID NO.:130, SEQ ID NO.:131, SEQ ID NO.:132 and SEQ ID NO.:133 or to a complement thereof and wherein the addition and/or deletion is located at a 5′ end and/or 3′ end of the nucleic acid sequence;
d) at least one member selected from the group consisting of an oligonucleotide comprising from 0 to 5 nucleotide addition and/or deletion to SEQ ID NO.:134, SEQ ID NO.:135, SEQ ID NO.:136, SEQ ID NO.:137, SEQ ID NO.:138, SEQ ID NO.:139, SEQ ID NO.:140, SEQ ID NO.:141, SEQ ID NO.:142, SEQ ID NO.:143, SEQ ID NO.144, SEQ ID NO.:145, SEQ ID NO.:146, SEQ ID NO.:147, SEQ ID NO.:148, SEQ ID NO.:149, SEQ ID NO.:150, SEQ ID NO.:151, SEQ ID NO.:152, SEQ ID NO.:201, SEQ ID NO.:202, SEQ ID NO.:203, SEQ ID NO.:204, SEQ ID NO.:205, SEQ ID NO.:206, SEQ ID NO.:207 and SEQ ID NO.:208 or to a complement thereof and wherein the addition and/or deletion is located at a 5′ end and/or 3′ end of the nucleic acid sequence;
e) at least one member selected from the group consisting of an oligonucleotide comprising from 0 to 5 nucleotide addition and/or deletion to SEQ ID NO.:153 and SEQ ID NO.:154 or to a complement thereof and wherein the addition and/or deletion is located at a 5′ end and/or 3′ end of the nucleic acid sequence;
f) at least one member selected from the group consisting of an oligonucleotide comprising from 0 to 5 nucleotide addition and/or deletion to SEQ ID NO.:155, SEQ ID NO.:156 and SEQ ID NO.:157 or to a complement thereof and wherein the addition and/or deletion is located at a 5′ end and/or 3′ end of the nucleic acid sequence;
g) at least one member selected from the group consisting of an oligonucleotide comprising from 0 to 5 nucleotide addition and/or deletion to SEQ ID NO.:158, SEQ ID NO.:209 and SEQ ID NO.:225, SEQ ID NO.:226, SEQ ID NO.:227, SEQ ID NO.:228 and SEQ ID NO.:229 or to a complement thereof and wherein the addition and/or deletion is located at a 5′ end and/or 3′ end of the nucleic acid sequence;
h) at least one member selected from the group consisting of an oligonucleotide comprising from 0 to 5 nucleotide addition and/or deletion to SEQ ID NO.:159 and SEQ ID NO.:160 or to a complement thereof and wherein the addition and/or deletion is located at a 5′ end and/or 3′ end of the nucleic acid sequence;
i) at least one member selected from the group consisting of an oligonucleotide comprising from 0 to 5 nucleotide addition and/or deletion to SEQ ID NO.:161 or to a complement thereof and wherein the addition and/or deletion is located at a 5′ end and/or 3′ end of the nucleic acid sequence;
j) at least one member selected from the group consisting of an oligonucleotide comprising from 0 to 5 nucleotide addition and/or deletion to SEQ ID NO.:162 or to a complement thereof and wherein the addition and/or deletion is located at a 5′ end and/or 3′ end of the nucleic acid sequence;
k) at least one member selected from the group consisting of an oligonucleotide comprising from 0 to 5 nucleotide addition and/or deletion to SEQ ID NO.:163 and SEQ ID NO.:164 or to a complement thereof and wherein the addition and/or deletion is located at a 5′ end and/or 3′ end of the nucleic acid sequence;
l) at least one member selected from the group consisting of an oligonucleotide comprising from 0 to 5 nucleotide addition and/or deletion to SEQ ID NO.:165, SEQ ID NO.:166, SEQ ID NO.:167, SEQ ID NO.:168, SEQ ID NO.:169, SEQ ID NO.:170, SEQ ID NO.:171, SEQ ID NO.:172, SEQ ID NO.:173, SEQ ID NO.:174, SEQ ID NO.:175, SEQ ID NO.:176, SEQ ID NO.:177 and SEQ ID NO.:178 or to a complement thereof and wherein the addition and/or deletion is located at a 5′ end and/or 3′ end of the nucleic acid sequence;
m) at least one member selected from the group consisting of an oligonucleotide comprising from 0 to 5 nucleotide addition and/or deletion to SEQ ID NO.:179, SEQ ID NO.:180, SEQ ID NO.:181, SEQ ID NO.:182, SEQ ID NO.:183, SEQ ID NO.:210, SEQ ID NO.:211, SEQ ID NO.:212, SEQ ID NO.:213 and SEQ ID NO.:214 or to a complement thereof and wherein the addition and/or deletion is located at a 5′ end and/or 3′ end of the nucleic acid sequence;
n) at least one member selected from the group consisting of an oligonucleotide comprising from 0 to 5 nucleotide addition and/or deletion to SEQ ID NO.:184, SEQ ID NO.:185, SEQ ID NO.:186, SEQ ID NO.:187 and SEQ ID NO.:188 or to a complement thereof and wherein the addition and/or deletion is located at a 5′ end and/or 3′ end of the nucleic acid sequence;
wherein each oligonucleotide is attached to a solid support and wherein each oligonucleotide is located at an addressable position.

95. A method for the diagnosis of a respiratory infection in an individual in need, the method comprising detecting the presence or absence of a pathogen from a sample obtained from the individual with oligonucleotides capable of specific binding with a genetic material of a respiratory virus species, wherein said oligonucleotides are selected from the group consisting of:
a) an oligonucleotide comprising or consisting of the sequence selected from the group consisting of SEQ ID NO.:78, SEQ ID NO.:79, SEQ ID NO.:80, SEQ ID NO.:81, SEQ ID NO.:82, SEQ ID NO.:83, SEQ ID NO.:84, SEQ ID NO.:85, SEQ ID NO.:86, SEQ ID NO.:87, SEQ ID NO.:88, SEQ ID NO.:89, SEQ ID NO.:90, SEQ ID NO.:91, SEQ ID NO.:92, SEQ ID NO.:93, SEQ ID NO.:94, SEQ ID NO.:95, SEQ ID NO.:96, SEQ ID NO.:97, SEQ ID NO.:98, SEQ ID NO.:99, SEQ ID NO.:100, SEQ ID NO.:101, SEQ ID NO.:102 and SEQ ID NO.:103;
b) an oligonucleotide comprising or consisting of the sequence selected from the group consisting of SEQ ID NO.:106, SEQ ID NO.:107, SEQ ID NO.:108, SEQ ID NO.:109, SEQ ID NO.:110, SEQ ID NO.:111, SEQ ID NO.:112, SEQ ID NO.:113, SEQ ID NO.:114, SEQ ID NO.:115, SEQ ID NO.:116, SEQ ID NO.:117, SEQ ID NO.:118, SEQ ID NO.:119, SEQ ID NO.:120, SEQ ID NO.:121, SEQ ID NO.:122, SEQ ID NO.:123, SEQ ID NO.:124, SEQ ID NO.:125, SEQ ID NO.:126, SEQ ID NO.:127, SEQ ID NO.:128, SEQ ID NO.:129, SEQ ID NO.:130, SEQ ID NO.:131, SEQ ID NO.:132, SEQ ID NO.:133, SEQ ID NO.:134, SEQ ID NO.:135, SEQ ID NO.:136, SEQ ID NO.:137, SEQ ID NO.:138, SEQ ID NO.:139, SEQ ID NO.:140, SEQ ID NO.:141, SEQ ID NO.:142, SEQ ID NO.:143, SEQ ID NO.144, SEQ ID NO.:145, SEQ ID NO.:146, SEQ ID NO.:147, SEQ ID NO.:148, SEQ ID NO.:149, SEQ ID NO.:150, SEQ ID NO.:151, SEQ ID NO.:152, SEQ ID NO.:153, SEQ ID NO.:154, SEQ ID NO.:155, SEQ ID NO.:156, SEQ ID NO.:157, SEQ ID NO.:158, SEQ ID NO.:159, SEQ ID NO.:160, SEQ ID NO.:161, SEQ ID NO.:162, SEQ ID NO.:163, SEQ ID NO.:164, SEQ ID NO.:165, SEQ ID NO.:166, SEQ ID NO.:167, SEQ ID NO.:168, SEQ ID NO.:169, SEQ ID NO.:170, SEQ ID NO.:171, SEQ ID NO.:172, SEQ ID NO.:173, SEQ ID NO.:174, SEQ ID NO.:175, SEQ ID NO.:176, SEQ ID NO.:177, SEQ ID NO.:178, SEQ ID NO.:179, SEQ ID NO.:180, SEQ ID NO.:181, SEQ ID NO.:182, SEQ ID NO.:183, SEQ ID NO.:184, SEQ ID NO.:185, SEQ ID NO.:186, SEQ ID NO.:187, SEQ ID NO.:188, SEQ ID NO.:194, SEQ ID NO.:195, SEQ ID NO.:196, SEQ ID NO.:197, SEQ ID NO.:198, SEQ ID NO.:199, SEQ ID NO.:200, SEQ ID NO.:201, SEQ ID NO.:202, SEQ ID NO.:203, SEQ ID NO.:204, SEQ ID NO.:205, SEQ ID NO.:206, SEQ ID NO.:207, SEQ ID NO.:208, SEQ ID NO.:209, SEQ ID NO.:210, SEQ ID NO.:211, SEQ ID NO.:212, SEQ ID NO.:213, SEQ ID NO.:214, SEQ ID NO.:225, SEQ ID NO.:226, SEQ ID NO.:227, SEQ ID NO.:228, SEQ ID NO.:229;
c) the oligonucleotide of a) comprising from 0 to 5 nucleotide addition or deletion at a 5′ end thereof;
d) the oligonucleotide of b) comprising from 0 to 5 nucleotide addition, deletion or combination of addition and deletion at a 5′ end and/or 3′ end thereof; and
e) a complement of any one of the above.


SEQUENCE LISTING (229 sequences)

SEQ ID NO: 1 - DNA, 912 bp, InfluenzaA A/Beijing/262/95
catcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacgggratccaaataacatggacaaagcagttaaactgtataggaagctcaagagggagataacattccatggggccaaagaaatctcactcagttattctgctggtgcacttgccagttgtatgggcctcatatacaacaggatgggggatgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcaagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatg

SEQ ID NO: 2 - DNA, 895 bp, InfluenzaA/new caledonia/20/99
tcaaagccgagatcgcacagagacttgaagatgtctttgctggaaagaataccgatcttgaggctctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaatggggatccaaataatatggacagagcagttaaactgtatcgaaagcttaagagggagataacattccatggggccaaagaaatagcactcagttattctgctggtgcacttgccagttgtatgggactcatatacaacaggatgggggctgtgaccaccgaatcagcatttggccttatatgcgcaacctgtgaacagattgccgactcccagcataagtctcataggcaaatggtaacaacaaccaacccattaataagacatgagaacagaatggttctggccagcactacagctaaggctatggagcaaatggctggatcgagtgaacaagcagctgaggccatggaggttgctagtcaggccaggcagatggtgcaggcaatgagagccattgggactcatcctagctctagcactggtctgaaaaatgatctccttgaaaatttgcaggcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatg

SEQ ID NO: 3 - DNA, 887 bp, InfluenzaA/fujian/7837/03
gagatcgcgcagagacttgaagatgtctttgctgggaaaaacacagatcttgaggctctcatggaatggctaaagacaagaccaattctgtcacctctgactaaggggattttagggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaatagaatgggggctgtaaccactgaagtagcatttggcctggtatgtgcaacatgtgaacagattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggaaattgctagtcaggccaggcaaatggtgcaggcaatgagagccgttgggactcatcctagctccagtactggtctaagagatgatcttcttgaaaatttgcagacctatcagaaacgaatgggggtgcagatgcaacgattcaagtgacccgcttgttgttgccgcgagtatcattggaatcttgcacttgatattgtggattcttgatcgtctttttttcaaatgcgtctatcgactcttcaaacacggccttaaaagaggcccttctacggaaggagtacctgagtctatgagggaagaatatcgaaaggaacagcagaatgctgtggatgctgacgaca

SEQ ID NO: 4 - DNA, 894 bp, InfluenzaA/panama/5502/98
caaagccgaaatcgcgcagagacttgaagatgtctttgctgggaaaaacacagatcttgaggctctcatggaatggctaaagacaagaccaatcctgtcacctctgactaaggggattttggggtttgtgttcacgctcaccgtgcccagtgagcggggactgcagcgtagacgctttgtccaaaatgccctcaatgggaatggggatccaaataacatggacaaagcagttaaactgtatagaaaacttaagagggagataacattccatggggccaaagaaatagcactcagttattctgctggtgcacttgccagttgcatgggcctcatatacaataggatgggggctgtaaccaccgaagtggcatttggcctggtatgtgcaacatgtgaacagattgctgactcccagcacaggtcccataggcaaatggtggcaacaaccaatccattaataaaacatgagaacagaatggttttggccagcactacagctaaggctatggagcaaatggctggatcaagtgagcaggcagcggaggccatggagattgctagtcaggccaggcaaatggtgcaggcaatgaggaccgttgggactcatcctagctccagtactggtctaagagatgatcttcttgaaaatttgcagacctatcagaaacgaatgggggtgcagatgcaacgattcaaatgacccgcttgttgttgctgcgagtatcattgggatcttgcacttgatactgtggattcttgatcgtctttttttcaaatgcatctatcgactcttcagacacggcctgaaaagagggccttctacggaaggagtacctgagtctatgagggaagaatatcgaaaggaacagcagaatgctgtggatgctgacgaca

SEQ ID NO: 5 - DNA, 895 bp, InfluenzaA/sydney/5/97
tcaaagccgaaatcgcgcagagacttgaagatgtctttgctgggaaaaacacagatcttgaggctctcatggaatggctaaagacaagaccaatcctgtcacctctgactaaggggattttggggtttgtgttcacgctcaccgtgcccagtgagcgaggactgcagcgtagacgctttgtccaaaatgccctcaatgggaatggggatccaaataacatggacaaagcagttaaactgtatagaaaacttaagagggagataacattccatggggccaaagaaatagcactcagttattctgctggtgcacttgccagttgcatgggcctcatatacaataggatgggggctgtaaccaccgaagtggcatttggcctggtatgtgcaacatgtgaacagattgctgactcccagcacaggtctcataggcaaatggtggcaacaaccaatccattaataagacatgagaacagaatggttttggccagcactacagctaaggctatggagcaaatggctggatcaagtgagcaggcagcggaggccatggagattgctagtcaggccaggcaaatggtgcaggcaatgagagccgttgggactcatcctagctccagtactggtctaagagatgatcttcttgaaaatttgcagacctatcagaaacgaatgggggtgcagatgcaacgattcaagtgacccgcttgttgttgctgcgagtatcattgggatcttgcacttgatattgtggattcttgatcgtctttttttcaaatgcatctatcgactcttcaaacacggcctgaaaagagggccttctacggaaggagtacctgagtctatgagggaagaatatcgaaaggaacagcagaatgctgtggatgctgacgaca

SEQ ID NO: 6 - DNA, 895 bp, InfluenzaA/wsn/33
tcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacagcaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccattgggactcatcctagctccagtgctggtctaaaagatgatcttcttgaaaatttgcaggcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatg

SEQ ID NO: 7 - DNA, 912 bp, InfluenzaA CCRI-15615
cgttccatcaggccccctcaaagccgagatcgcgcagagacttgaagatgtctttgctgggaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaatagaatgggggctgtaaccactgaagtggcatttggcctggtatgtgcaacatgtgaacagattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggaaattgctagtcaggccaggcaaatggtgcaggcaatgagaaccgttgggactcatcctagctccagtactggtctaagagatgatcttcttgaaaatttgcagacctatcaaaaacgaatgggggtgcagatgcaacgattcaagtgacccgcttgttgttgccgcgagtatcattgggatcttgcacttgatattgtggattcttgatcgtctttttttcaaatgcgtctatcgactcttcaaacacggccttagaagaggcccttctacggaaggagtacctgagtctatgagggaagaatatcgaaaggaacagcagaatgctgtggatgctgacgaca

SEQ ID NO: 8 - DNA, 912 bp, InfluenzaA CCRI-15634; identical to SEQ ID NO: 7

SEQ ID NO: 9 - DNA, 912 bp, InfluenzaA CCRI-15707
cgttccatcaggccccctcaaagccgagatcgcgcagagacttgaagatgtctttgctgggaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaatagaatgggggctgtaaccactgaagtggcatttggcctggtatgtgcaacatgtgaacagattgctgactcccagcacaggtctcataggcaaatggtggcaacaaccaatccattaataaaacatgagaacagaatggttttggccagcactacagctaaggctatggagcaaatggctggatcaagtgagcaggcagcggaggccatggaaattgctagtcaggccaggcaaatggtgcaggcaatgagaaccgttgggactcatcctagctccagtactggtctaagagatgatcttcttgaaaatttgcagacctatcaaaaacgaatgggggtgcagatgcaacgattcaagtgacccgcttgttgttgccgcgagtatcattgggatcttgcacttgatattgtggattcttgatcgtctttttttcaaatgcgtctatcgactcttcaaacacggccttagaagaggcccttctacggaaggagtgcctgagtctatgagggaagaatatcgaaaggaacagcagaatgctgtggatgctgacgaca

SEQ ID NO: 10 - DNA, 895 bp, InfluenzaA/ws/33
tcaaagccgagatcgcacagagacttgaagatgtctttgcagggaagaacaccgatcttgaggctctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccattgggactcatcctagctccagtgctggtctaaaagatgatcttcttgaaaatttgcaggcctatcagaaacgaatgggggtgcagatgcaacgattcaagtgatcctctcgttattgcagcaaatatcattgggatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatg

SEQ ID NO: 11 - DNA, 912 bp, InfluenzaA/wuham/7244/98
cgttccatcaggccccctcaaagccgaaatcgcgcagagacttgaagatgtctttgctgggaaaaacacagatcttgaggctctcatggaatggctaaagacaagaccaatcctgtcacctctgactaaggggattttggggkttgtgttcacgctcaccgtgcccagtgagcgaggactgcagcgtagacgctttgtccaaaatgccctcaatgggaatggggatccaaataacatggacagagcagttaaactgtatagaaaacttaagagggagataacattccatggggccaaagaaatcgcactcagttattctgctggtgcacttgccagttgcatgggcctcatatacaacaggatgggggctgtaaccactgaagtggcctttggcctggtatgtgcaacatgtgaacagattgctgactcccagcacaggtctcataggcaaatggtggcaacaaccaatccattaataaaacatgagaacagaatggttttggccagcactacggctaaggctatggagcaaatggctggatcaagtgagcaggcagcggaggccatggagattgctagtagggccaggcaaatggtgcaggcaatgagagccgttgggactcatcctagctccagtactggtctaagagatgatcttcttgaaaatttgcagacctatcagaaacgaatgggagtgcagatgcaacgattcaagtgacccgcttgttgttgctgcgagtatcattgggatcttgcacttgatattgtggattcttgatcgtctttttttcaaatgcatctatcgactcttcaaacacggcctgaaaagagggccttctacggaaggagtacctgagtctatgagggaagaatatcgaaaggaacagcagaatgctgtggatgctgacgaca

SEQ ID NO: 12 - DNA, 887 bp, InfluenzaA CCRI-16130
gagatcgcacagagacttgaagatgtctttgctgggaaaaacaccgatcttgaggctctcatggaatggctaaagacaagaccaatcctgtcacctctgactaaggggattttgggatttgtattcacgctcaccgtgcccagtgagcgaggactgcagcgtagacgctttgtccaaaatgccctcaatgggaatggggatccaaataacatggacagagcagttaaactgtatagaaagcttaagagggagataacattccatggggccaaagaaatagcgctcagttattctgctggtgcacttgccagttgtatgggcctcatatacaacaggatgggggctgtgaccactgaagtggtcttaggcctggtatgtgcaacctgtgaacagattgctgactcccagcataggtctcataggcaaatggtgacaacaaccaatccactaataagacatgagaacagaatggttctggccagcactacagctaaggctatggagcaaatggctggatcgagtgagcaagcagcagaggccatggaggttgctagtcaggccaggcaaatggtgcaggcaatgagagttattgggactcatcctagctccagtgctggtctaaaaaatgatcttcttgaaaatttgcaggcctatcagaaacgaatgggggtgcagatgcaacgattcaagtgaccctcttgttgttgccgcgagtatcattgggatcttgcacttgatattgtggattcttgatcatctttttttcaaatgcatttatcgcttctttaaacacggtctgaaaagagggccttctacggaaggagtaccagagtctatgagggaagaatatcgaaaggaacagcagagtgctgtggattctgacgata

SEQ ID NO: 13 - DNA, 887 bp, InfluenzaA CCRI-16240
gagatcgcgcagagacttgaagatgtctttgctggaaaaaacacagatcttgaggctctcatggaatggctaaagacaagaccaattctgtcacctctgactaaggggattttggggtttgtgttcacgctcaccgtgcccagtgagcgaggactgcagcgtagacgctttgtccaaaatgccctcaatgggaatggagatccaaataacatggacaaagcagttaaactgtataggaaacttaagagggagataacgttccatggggccaaagaaatagctctcagttattctgctggtgcacttgccagttgcatgggcctcatatacaataggatgggggctgtaaccactgaagtggcatttggcctggtatgtgcaacatgtgagcagattgctgactcccagcacaggtctcataggcaaatggtggcaacaaccaatccattaataagacatgagaacagaatggttttggccagcactacagctaaggctatggagcaaatggctggatcaagtgagcaggcagcggaggccatggagattgctagtcaggccaggcagatggtgcaggcaatgagagccattgggactcaccctagttccagtactggtctaagagatgatcttcttgaaaatttgcagacctatcagaaacgaatgggggtgcagatgcaacgattcaagtgacccgcttgttgttgccgcgaatatcattgggatcttgcacttgatattgtggattcttgatcgtctttttttcaaatgcgtttatcgactcttcaaacacggccttaaaagaggcccttctacggaaggagtacctgagtctatgagggaagaatatcgaaaggaacagcagaatgctgtggatgctgacgaca

SEQ ID NO: 14 - DNA, 890 bp, InfluenzaA CCRI-16241
tcagcatccacagcattctgctgttcctttcgatattcttccctcatagactcaggtactccttccgtagaagggcctcttttaaggccgtatttgaggagtcggtagacgcatttgaaaaaaagacgatcaagaatccacaatatcaagtgcaagatcccaatgatattcgcggcaacaacaagcgggtcacttgaatcgttgcatctgcacccccattcgtttctgataggtctgcaaattttcaagaagatcatctcttagaccagtactggaactaggatgagtcccaatggctctcattgcctgcaccatctgtctggcctgactagcaatctccatggcctccgctgcctgctcacttgatccagccatttgctccatagccttagctgtagtgctggccaaaaccattctgttctcatgtcttattaatggattggttgttgccaccatttgcctatgagacctgtgctgggagtcagcaatctgctcacatgttgcacataccaggccaaatgccacttcagtggttacagcccccatcctattgtatatgaggcccatgcaactggcaagtgcaccagcagaataactgagagctatttctttggccccatggaacgttatctccctcttaagtttcctatacagtttaactgctttgtccatgttatttggatctccattcccattgagggcattttggacaaagcgtctacgctgcagtcctcgctcactgggcacggtgagcgtgaacacaaaccccaaaatccccttagtcagaggtgacagaattggtcttgtctttagccattccatgagagcctcaagatctgtgtttttcccagcaaagacatcttcaagtctctgcgcgatctcggctttga

SEQ ID NO: 15 - DNA, 955 bp, InfluenzaA/panama/5502/98cloned
tcgaaacgtacgttctctctatcgttccatcaggccccctcaaagccgaaatcgcgcagagacttgaagatgtctttgctgggaaaaacacagatcttgaggctctcatggaatggctaaagacaagaccaatcctgtcacctctgactaaggggattttggggtttgtgttcacgctcaccgtacccagtgagcggggactgcagcgtagacgctttgtccaaaatgccctcaatgggaatggggatccaaataacatggacaaagcagttaaactgtatagaaaacttaagagggagataacattccatggggccaaagaaatagcactcagttattctgctggtgcacttgccagttgcatgggcctcatatacaataggatgggggctgtaaccaccgaagtggcatttggcctggtatgtgcaacatgtgaacagattgctgactcccagcacaggtcccataggcaaatggtggcaacaaccaatccattaataaaacatgagaacagaatggttttggccagcactacagctaaggctatggagcaaatggctggatcaagtgagcaggcagcggaggccatggagattgctagtcaggccaggcaaatggtgcaggcaatgaggaccgttgggactcatcctagctccagtactggtctaagagatgatcttcttgaaaatttgcagacctatcagaaacgaatgggggtgcagatgcaacgattcaaatgacccgcttgttgttgctgcgagtatcattgggatcttgcacttgatactgtggattcttgatcgtctttttttcaaatgcatctatcgactcttcagacacggcctgaaaagagggccttctacggaaggagtacctgagtctatgagggaagaatatcgaaaggaacagcagaatgctgtggatgctgacgacagtcattttgtcagcatagagc

SEQ ID NO: 16 - DNA, 733 bp, human Respiratory Syncytial virus Long98-02
agaagatgctaatcataaattcactgggttaataggtatgttatatgctatgtctaggttaggaagagaagacaccataaaaatactcagagatgcgggatatcatgtaaaagcaaatggagtagatgtaacaacacatcgtcaagacatcaatgggaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgattctcctgattgtgggatgataatattatgtatagcagcattagtaataaccaaattggcagcaggggatagatctggtcttacagccgtgattaggagagctaataatgtcctaaaaaatgaaatgaaacgttacaaaggcttactacccaaggatatagccaacagcttctatgaagtgtttgaaaaacatccccactttatagatgtttttgttcattttggtatagcacaatcttccaccagaggtggcagtagagttgaagggatttttgcaggattgtttatgaatgcctatggtgcagggcaagtaatgctacggtggggagtcttagcaaaatcagttaaaaatattatgttaggacatgctagtgtgcaagcagaaatggaacaagttgttgaggtttatgaatatgcccaaaaattgggtggagaagca

SEQ ID NO: 17 - DNA, 733 bp, human Respiratory Syncytial virus CCRI-15552
agaagatgctaatcataaattcactggggtaataggtatgttatatgctatgtccagattaggaagagaagacaccataaaaatactcagagatgcgggatatcatgtaaaagcaaatggagtggatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtggctccagaatacaggcatgactctcctgattgtggaatgataatattatgtatagcagcattagtaataaccaaattagcagcaggggatagatctggtcttacagctgtgattaggagagctaataatgttctaaaaaatgaaatgaaacgttataaaggcttactacccaaggatatagccaacagcttctatgaagtgtttgaaaaatatcctcactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgctaggacacgctagtgtgcaagcagaaatggaacaagttgtggaagtttatgaatatgcccaaaaattgggtggagaagca

SEQ ID NO: 18 - DNA, 692 bp, human Respiratory Syncytial virus B wv/14617/85
tgaagatgcaaatcataaattcacaggattaataggtatgttatatgctatgtccaggttaggaagggaagacactataaagatacttaaagatgctggatatcatgttaaagctaatggagtagatataacaacatatcgtcaagatataaatggaaaggaaatgaaattcgaagtattaacattatcaagcttgacatcagaaatacaagtcaatattgagatagaatctagaaaatcctacaaaaaaatgctaaaagagatgggagaagtggctccagaatataggcatgattctccagactgtgggatgataatactgtgtatagcagcacttgtaataaccaaattagcagcaggagacagatcaggtcttacagcagtaattaggagggcaaacaatgtcttaaaaaatgaaataaaacgctacaagggtctcataccaaaggatatagctaacagtttttatgaagtgtttgaaaaacaccctcatcttatagatgtttttgtgcactttggcattgcacaatcatcaacaagagggggtagtagagttgaaggaatctttgcaggattgtttatgaatgcctatggttcagggcaagtaatgctaagatggggagttttagccaaatctgtaaaaaatatcatgctaggtcatgctagtgtccaggcagaaatggagcaagttg

SEQ ID NO: 19 - DNA, 733 bp, human Respiratory Syncytial virus RSV/B 9320
agaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagca

SEQ ID NO: 20 - DNA, 736 bp, humanRespiratorySyncytialvirus CCRI-15595
ccaacttctgtgcatactcatagacttccacaacttgctccatttctgcctggacactagcatgtcctagcatgatattttttacagatttggctaaaactccccatcttagcattacttgccctgaaccataggcattcataaataatcctgcaaagattccttcaactctactaccccctcttgtggatgattgtgcaatgccaaagtgcacaaaaacatctataagatgagggtgtttttcaaacacttcataaaaactgttagctatatcctttggtatgaggcccttgtagcgttttatttcgttttttaagacattgtttgccctcctaattactgctgtaagacctgatctatctcctgctgctaatttggttattacaagtgcagctatacacagtattatcatcccacagtctggagaatcatgcctatattctggagccacttctcccatctcttttagcatttttttgtaggactttctagattctatctcaatattgacttgtatttctgatgtcaagcttgataatgttaatacttcgaatttcatttcctttccatttatatcttgacgatatgttgttatatctactccattagctttaacatgatatccagcatctttaagtatctttatagtgtcttcccttcctaacctggacatagcatataacatacctattaatcctgtgaatttatgatttgcatcttcagtgattaataactg

SEQ ID NO: 21 - DNA, 775 bp, humanRespiratorySyncytialvirusCCRI-15552cloned
caatatatggtagaatcctgcttctccacccaatttttgggcatattcataaacttccacaacttgttccatttctgcttgcacactagcgtgtcctagcataatatttttaactgattttgctaagactccccaccgtaacatcacttgccctgcaccataggcattcataaacaatcctgcaaaaatcccttcaactctactgccacctctggtagaagattgtgctataccaaaatgaacaaaaacatctataaagtgaggatatttttcaaacacttcatagaagctgttggctatatccttgggtagtaagcctttataacgtttcatttcattttttagaacattattagctctcctaatcacagctgtaagaccagatctatcccctgctgctaatttggttattactaatgctgctatacataatattatcattccacaatcaggagagtcatgcctgtattctggagccacctctcccatttcttttagcatttttttgtaggattttctagattctatctcaatgttgatttgaatttcagttgttaagcttgccaatgttaacacttcaaatttcatttcttttccattaatgtcttgacgatgtgttgttacatctactccatttgcttttacatgatatcccgcatctctgagtatttttatggtgtcttctcttcctaatctggacatagcatataacatacctattaccccagtgaatttatgattagcatcttctgtgattaataacatgccacataaa

SEQ ID NO: 22 - DNA, 990 bp, humanmetapneumovirusCan97-83(a2)cloned
gagtctcagtacacaataaagagagatgtaggcacaacaacagcagtgacaccctcatcattgcaacaagaaataacactattgtgtggagaaattctatatgctaagcatgctgattacaaatatgctgcagaaataggaatacaatatattagcacagctctaggatcagagagagtacagcagattctaagaaactcaggcagtgaagtccaagtggttttaaccagaacgtactccttggggaaagttaaaaacaacaaaggagaagatttacagatgttagacatacacggagtagagaaaagctgggtggaagagatagacaaagaagcaagaaaaacaatggcaactttgcttaaagaatcatcaggcaatattccacaaaatcagaggccttcagcaccagacacacctataatcttattatgtgtaggtgccttaatatttaccaaactagcatcaactatagaagtgggattagagaccacagtcagaagagctaaccgtgtactaagtgatgcactcaaaagataccctaggatggacataccaaaaatcgctagatctttctatgatttatttgaacaaaaagtgtattacagaagtttgttcattgagtatggcaaagcattaggctcatcctctacaggcagcaaagcagaaagtttattcgttaatatattcatgcaagcttacggtgctggtcaaacaatgctgaggtggggagtcattgccaggtcatctaacaatataatgttaggacatgtatctgtccaagctgagttaaaacaagtcacagaagtctatgacctggtgcgagaaatgggccctgaatctgggctcctacatttaaggcaaagcccaaaagctggactgttatcactagccaattgtcccaactttgcaagtgttgttctcggcaatgcctcaggcttaggcataataggtatgtatcgcgggagagtgccaaacacagaattattttca

SEQ ID NO: 23 - DNA, 402 bp, humanEnterovirus CCRI-15531
taccccggactgagtatcaataagctgcttacgcggctgaaggagaaaacgtccgttacccggccaactacttcgagaagcccagtactaccatgaaggatgcgcagtgtttcgttcagcacaaccccagtgtagatcaggccgatgagtcaccgcaatccccacgggtgaccgtggcggtggctgcgctggcggcctgcccatggggcaacccatgggacgcttcaattccgacatggtgcgaagagtctattgagctagttggtagtcctccggcccctgaatgcggctaatcccaactgcggagcagacacccacaaaccagtgggcagtttgtcgtaacgggcaactctgcagcggaaccgactactttgggtgtccgtgtttcctattattcctaca

SEQ ID NO: 24 - DNA, 402 bp, humanEnterovirus CCRI-15527
taccccggactgagtatcaataggctgcttacgcggctgaaggagaaaacgttcgttacccggccaactacttcgagaagcccagtactaccatgaaggatgcgcagtgtttcgttcagcacaaccccagtgtagatcaggccgatgagtcaccgcaatccccacgggtgaccgtggcggtggctgcgctggcggcctgcccatggggcaacccatgggacgcttcaattccgacatggtgcgaagagtctattgagctagttggtagtcctccggcccctgaatgcggctaatcccaactgcggagcagacacccacaaaccagkgggcagtttgtcgtaacgggcaactctgcagcggaaccgactactttgggtgtccgtgtttcctattattcctaca

SEQ ID NO: 25 - DNA, 402 bp, humanEnterovirus CCRI-15515
ttccccggactgagtatcaataggctgctagcgcggccgaaggagaaaacgttcgttacccggccaattacttcgagaaacctagtaccaccatgaacgttgcgcagtgtttcgctccacacaaccccagtgtagatcaggccgatgagtcaccgcaatccccacgggtgaccgtggcggtggctgcgccggcggcctgcccatggggcaacccatgggacgcttcaatactgacatggtgtgaagagtctattgagctaattggtagtcctccggcccctgaatgcggctaatcctaactgcggagcagatacccacacaccagtgggcagtctgtcgtaacgggcaactctgcagcggaaccgactactttgggtgtccgtgtttccttttattcttata

SEQ ID NO: 26 - DNA, 402 bp, humanEnterovirus CCRI-15502
taccccggaccgagtatcaataggctgctcacgcggctgaaggagaaagtgttcgttacccgaccaattacttcgagaaacttagtaccgccatgaaggttgcgcagcgtttcgttccgcacaaccccagtgtagatcaggtcgatgagccaccgcataccccacgggtgaccgtggcggtggctgcgctggcggcctgcccatggggtaacccatgggacgcttcaatactgacatggtgtgaagagtctattgagctaattggtagtcctccggcccctgaatgcggctaatcctaactgcggagcagatacccacaaaccagtgggcagtctgtcgtaacgggcaactctgcagcggaaccgactactttgggtgtccgtgtttccttttattcttgta

SEQ ID NO: 27 - DNA, 402 bp, humanEchovirus16Harrington
taccccggactaagtatcaataagcggctaacgcggctgaaggagaaagcgtccgttacccggccaactacttcgggaaatctagtaacaccatgaaggttgcgcagtgtttcgctccgcacaaacccagtgtagatcaggtcgatgagtcaccgcattccccacaggcgactgtggcggtggctgcgttggcggcctgcccatggggcaacccatgggacgcttcaatactgacatggtgcgaagaatctactgagctagttggtagtcctccggcccctgaatgcggctaatcctaactgcggagcacatacccctaacccaaggggcagtgtgtcgtaacgggcaactctgcagcggaaccgactactttgggtgtccgtgtttccttttattcttata

SEQ ID NO: 28 - DNA, 402 bp, humanCoxsackievirusb1Conn-5
taccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctata

SEQ ID NO: 29 - DNA, 402 bp, humanEchovirus25JV-4
ttccccggaccgagtatcaatagactgctcacgcggttgaaggagaaaacgttcgttatccggctaactacttcgaaaaacctagtaacaccatgaaagttgcggagtgtttcactcagcacttccccagtgtagatcaggtcgatgagtcaccgcattcctcacgggcgaccgtggcggtggctgcgctggcggcctgcctatggggtgacccataggacgctctaatacagacatggtgcgaagagtctattgagctagttagtagtcctccggcccctgaatgcggataatcctaactgtggagcagatacccacgaaccagtgggcagtctgtcgtaacgggcaactccgcagcggaaccgactactttgggtgtccgtgtttccttttattccaaat

SEQ ID NO: 30 - DNA, 402 bp, humanEchovirus20JV-1
taccccggactgagtatcaatagactgctcacgcggttgaaggagaaagcgttcgttatccggccaactacttcgagaaacctagtaacaccgtgaaagttgcagagtgtttcgctcagcactaccccagtgtagatcaggtcgatgagtcaccgcattccccacgggcgaccgtggcggtggctgcgctggcggcctgcctacggggaaacccgtaggacgctctaatacagacatggtgcgaagagtctattgagctagttggtagtcctccggcccctgaatgcggctaatcctaactgcggagcacacacccccaagccagggggcagtgtgtcgtaacgggtaactctgcagcggaaccgactactttgggtgtccgtgtttcattttattcctatg

SEQ ID NO: 31 - DNA, 402 bp, humanEchovirus4Pesascek
ctccccggaccgagtatcaatagactgcttgcgcggttgaaggagaaaacgttcgttacccggccaactacttcgagaaacctagtaccaccatgaaagttgcggagtgtttcgctcagcactaccccagtgtagatcaggccgatgagtcaccgcgttccccacgggtgaccgtggcggtggctgcgctggcggcctgcctatggggcaacccataggacgctctaatacagacatggtgtgaagagtctattgagctagttggtgatcctccggcccctgaatgcggctaatcctaactgcggagcacacgctcacaagccagtgagtggtgtgtcgtaatgggtaactccgcagcggaaccgactactttgggtgtccgtgtttccttttaacttcatt

SEQ ID NO: 32 - DNA, 402 bp, humanEchovirus19Burke
ttccccggaccaagtatcaatagactgctcacgcggttgaaggagaaagcgttcgttaaccggctaactacttcgagaaacccagtaacaccatgaaagttgcagagtgtttcgctcagcactcccccagtgtggatcaggtcgatgagtcactgcgttccccacgggtgaccgtggcagtggctgcgttggcggcctgcctgtggggcaacccacaggacgctctaatacggacatggtgtgaagagtctattgagctagttagtagtcctccggcccctgaatgcggctaatcctaactgtggagcacatgcccctaacccaaggggtggtgtgtcgtaatgggtaactccgcagcggaaccgactactttgggtgaccgtgtttccttttatttttaca

SEQ ID NO: 33 - DNA, 402 bp, humanEnterovirus CCRI-15561
ttccccggactgagtatcaataagctgctcgcgtggctgaaggagaaaacgttcgttatccggctaactacttcgggaaacctagtagcgccatgaaagttgcggagtgtttcgctcagcacttcccccgtgtagatcaggtcgatgagtcactgtaacccccacgggcgaccgtggcagtggctgcgttggcggcctgcctatggggtaacccataggacgctctaatacggacatggtgtgaagagtctattgagctagttagtagtcctccggcccctgaatgcggctaatcctaactgcggagcacataccctcaatccagggggtgatgtgtcgtaacgggtaactctgcagcggaaccgactactttgggtgtccgtgtttccttttattcttgta

SEQ ID NO: 34 - DNA, 358 bp, humanRhinovirus CCRI-15734
ggttgatgttgatatgctccaacagggcaaaaacaactgatatcgttatccgcaaagtgcctacacagagcctagtaggattctgaaagatctttggttggtcgttcagctgcatacccagcagtagaccttgcagatgaggctggacattccccactggtaacagtggtccagcctgcgtggctgcctgcacacctcttacgaggtgtgaagccaaggattggacagggtgtgaagagccgcgtgtgctcactttgagtcctccggcccctgaatgcggctaaccttaaacctgcagccatagctcataagccaatgagtttgtggtcgtaatgagtaattgcgggatgggaccgac

SEQ ID NO: 35 - DNA, 358 bp, humanRhinovirus CCRI-15732
ggttgacgttgatatgctccaacagggcaaaaacaactgatatcgttatccgcaaagtgcctacacagagcttagtaggattctgaaagatctttggttggtcgctcaactgcatacccagcagtagaccttgcagatgaggctggacattccccactggtaacagtggtccagcctgcgtggctgcctgcacacctcttatgaggtgtgaagccaaggattggacagggtgtgaagagccgcgtgtgctcactttgagtcctccggcccctgaatgcggctaaccttaaacctgcagccatggctcataaaccaatgagtttatggtcgtaatgagtaattgcgggatgggaccgac

SEQ ID NO: 36 - DNA, 357 bp, humanRhinovirus CCRI-15733
ggtcagcgttgatatgctctaacagggcaaaaacaacaatgatcgttatccgcaagatgcctacacagagcctagtagcactctagaagaattctggttggtcgctcagttgcaaacccagcaatagacctggcagatgaggctggacgttccccactggcgacagtggtccagcctgcgtggctgcctgcacactcctttggagtgtgaagccttaatttggacagggtgcgaagagccgcgtgtgctcatcttgaatcctccggcccctgaatgcggctaaccttaaacctgcagccattgtttgcaacccagcaaatatgtggtcgtaatgagtaattgcgggatgggaccgac

SEQ ID NO: 37 - DNA, 444 bp, humanEnterovirusCCRI-15531cloned
aagggtcaagcacttctgttaccccggactgagtatcaataagctgcttacgcggctgaaggagaaaacgtccgttacccggccaactacttcgagaagcccagtactaccatgaaggatgcgcagtgtttcgttcagcacaaccccagtgtagatcaggccgatgagtcaccgcaatccccacgggtgaccgtggcggtggctgcgctggcggcctgcccatggggcaacccatgggacgcttcaattccgacatggtgcgaagagtctattgagctagttggtagtcctccggcccctgaatgcggctaatcccaactgcggagcagacacccacaaaccagtgggcagtttgtcgtaacgggcaactctgcagcggaaccgactactttgggtgtccgtgtttcctattattcctacactggctgcttatggtgacaatta

SEQ ID NO: 38 - DNA, 718 bp, humanParainfluenzatype1 CCRI-15598
agtagacaaactttcaaatgtaggggttataatcaatgagggcaaattacttaagatagcaggatcttatgaatctagatacatagtgttaagcctggtaccttccattgacttacaagatggatgtgggacaactcaaattattcaatacaagaatttattaaatagacttctaattcctctgaaggatgccttggatcttcaggaatccctgataacaataaccaatgacaccactgtgacaaatgacaatccacaaactagattctttggtgctgtcattggtaccatagcactaggagtagccacagctgctcagataactgcgggaattgcactagccgaagcacgagaagccaggaaggacatagcactaataaaagattccatagtcaagacacacaattctgtagaattcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacggcattcagctccaatcttgggactataggagaaaaaagtcttaccttgcaggcattatcatctctctactctgctaatataacagaaattctaagtacaattaaaaaagataaatcagatatatatgacatcatttacactgaa

SEQ ID NO: 39 - DNA, 717 bp, humanParainfluenzatype1 CCRI-15560
gtggacaaactttcaaatgtaggggttataatcaatgagggtaaattacttaagatagcaggatcttatgaatctagatacatagtgttaagcctggtaccttccattgacttacaagatggatgtggaacaacccaaattattcaatacaagaatttattaaatagacttctaattcctctgaaggatgccttggatcttcaggaatccctgataacaataaccaatgacaccactgtgacaaatgacaatccacaaactagattctttggtgctgtcattggtaccatagcactaggagtagccacagctgctcagataactgcgggaattgcactagccgaagcacgagaagccaggaaggacatagcactaataaaagattccatagtcaagacacacaattctgtagaattcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacagcattcagctccaatcttgggactataggagaaaaaagtcttaccttgcaggcattatcatctctctactctgctaatataacagaaattctaagtacaattaaaaaagataaatcagatatatatgacatcatttacactgaa

SEQ ID NO: 40 - DNA, 698 bp, humanParainfluenzatype1(HA-2)C-35
  misc_feature (240)..(240): n can be either a/g/c or t
tgggggttataatcaatgagggcaaattactcaaaatagcaggatcttatgaatctagatacatagtgttaagcctggtgccttcaattgacctagaagatggatgtggaacaactcaaattattcaatacaagaatttattaaatagacttctaattcctctgaaggatgccttagatcttcaggaatccctgataacaataactaatgacaccactgtgacaaatgacaatccacaanctagattcttcggtgctgttattggtaccatagcactaggagtagccacagctgcccagataactgcaggcattgcattagctgaagcacgagaagccaggaaggacatagcactaatcaaagattctataatcaagacacacaattctgtagaactcattcaaagaggtataggagaacagataattgcattaaagacattacaagattttgtaaataatgagataagacctgcaataggagaactaaggtgtgaaaccacggcattgaaactagggatcaagctcacccaacactactctgaattagcaacagcattcagctccaatcttgggactataggagaaaaaagtcttaccttgcaggcattatcatctctctactctgctaatataacagaaatactaagtacaattaaaaaggataaatcagatatatatgacattatttatactgaa

SEQ ID NO: 41 - DNA, 718 bp, humanParainfluenzatype1CCRI-15598cloned
agtagacaaactttcaaatgtaggggttataatcaatgagggcaaattacttaagatagcaggatcttatgaatctagatacatagtgttaagcctggtaccttccattgacttacaagatggatgtgggacaactcaaattattcaatacaagaatttattaaatagacttctaattcctctgaaggatgccttggatcttcaggaatccctgataacaataaccaatgacaccactgtgacaaatgacaatccacaaactagattctttggtgctgtcattggtaccatagcactaggagtagccacagctgctcagataactgcgggaattgcactagccgaagcacgagaagccaggaaggacatagcactaataaaagattccatagtcaagacacacaattctgtagaattcattcaaagaggtataggagaacagataattgcattaaagacattacaagattttgtaaatgacgagataagacctgcaataggagaactaaggtgtgagactacggcattgaaactagggatcaagctcacccaacactactctgaattagcaacggcattcagctccaatcttgggactgtaggagaaaaaagtcttaccttgcaggcattatcatctctctactctgctaatataacagaaattctaagtacaattaaaaaagataaatcagatatatatgacatcatttacactgaa

SEQ ID NO: 42 - DNA, 806 bp, humanParainfluenzatype2 CCRI-15641
gagaccaactactgaacataggggtcattcaatcaaagataagatcactcatgtactatactgatggtggtgctagctttattgttgttaaattgctacctaatcttcccccaagcaatggaacatgcaacattaccagtctagatgcatacaatgttaccctatttaaattactaacacccttgattgagaacctgagcaagatctccgctgttacagataccaaaacccgccaagaacgatttgcaggagtcgttgttggacttgctgcattaggagtagccacagccgcacaaataactgcagctgtagcaatagttaaagctaatgcaaatgctgccgcgataaacagtcttgcatcttcaattcaatcaaccaacaaggcagtatccgatgtgatagatgcatcaaaaacaattgcaactgcagttcaggcaatccaggatcacatcaatggagctattgttaatgggataacatctgcatcatgccgtgctcatgatgcactcattgggtcaatattaaatctttatctcactgagcttaccacaatatttcacaatcaaataacaaaccctgcgctgacaccgctctccatccaagctttaagaattctcctcggtagcaccttgccaattgtcattgagtccaaactcaacacaaacctcaacacagcagagctgctcagctccggactgttaactggtcaaataatttcaatttccccaatgtacatgcaaatgctaattcaaatcaatgttccgacatttataatgcaacccggtgcgaaggtaattgatctaatt

SEQ ID NO: 43 - DNA, 806 bp, humanParainfluenzatype2Greer
gagatcaactacttaatataggggtcattcaatcaaagataagatcactcatgtactatactgatggtggtgctagctttattgttgtaaaattgctacctaatcttcccccaagcaatggaacatgcaacatcaccagyctagatgcatataatgttaccctatttaagttactaacacccctgattgagaacctgagtaaaatttccactgttacagataccaaaacccgccaagaacgatttgcaggagtagttgttggacttgctgcattaggagtagccacagccgcacaaataactgcagctgtagcaatagtgaaagctaatgcaaatgctgctgcgataaacaatcttgcatcttcaattcaatccaccaacaaggcagtatccgatgtgatagatgcatcaagaacaattgcaaccgcagttcaagcaattcaggatcacatcaatggagctattgttaatgggataacatctgcatcatgccgtgcccatgatgcactcattgggtcaatattaaatctttatctcactgagcttaccacaatatttcataatcaaataacaaaccctgcgctgacaccactctccatccaagctttaagaatcctcctcggtagcaccttgccaattgtcattgagtccaaactcaacacaaacttcaacacagcagagctgctcagttccggactgttaactggtcaaataatttccatttccccaatgtacatgcaaatgctaattcaaatcaatgttccgacatttataatgcaacccggtgcgaaggtaattgatctaatt

SEQ ID NO: 44 - DNA, 806 bp, humanParainfluenzatype2CCRI-15641cloned
gagaccaactactgaacataggggtcattcaatcaaagataagatcactcatgtactatactgatggtggtgctagctttattgttgttaaattgctacctaatcttcccccaagcaatggagcatgcaacattaccagtctagatgcatacaatgttaccctatttaaattactaacacccttgattgagaacctgagtaagatctccgctgttacagataccaaaacccgccaagaacgatttgcaggagtcgttgttggacttgctgcattaggagtagccacagccgcacaaataactgcagctgtagcaatagttaaagctaatgcaaatgctgccgcgataaacagtcttgcatcttcaattcaatcaaccaacaaggcagtatccgatgtgatagatgcatcaaaaacaattgcaactgcagttcaggcaatccaggatcacatcaatggagctattgttaatgggataacatctgcatcatgccgtgctcatgatgcactcattgggtcaatattaaatctttatctcactgagcttaccacaatatttcacaatcaaataacaaaccctgcgctgacaccgctctccatccaagctttaagaattctcctcggtagcaccttgccaattgtcattgagtccaaactcaacacaaacctcaacacagcagagctgctcagctccggactgttaactggtcaaataatttcaatttccccaatgtacatgcaaatgctaattcaaatcaatgttccgacatttataatgcaacccggtgcgaaggtaattgatctaatt

SEQ ID NO: 45 - DNA, 1060 bp, humanParainfluenzatype3 CCRI-15672
caatactgctaattattacaaccatgattatggcatctttctgccaaatagatatcacaaaactacagcatgtaggtgtattggttaacagtcccaaagggatgaagatatcacaaaactttgaaacaagatatctaattttgagcctcataccaaaaatagaagattctaactcttgtggtgaccaacagatcaagcaatacaagaggttattggatagactgatcattcctttatatgatggattaagattacagaaggatgtgatagtgtccaatcaagaatccaatgaaaacactgaccccagaacaaaacgattctttggaggggtaattggaactattgctctgggagtggcaacctcagcacaaattacagcggcagttgctctggttgaagccaagcaggcaagatcagacattgaaaaactcaaggaagcaatcagggacacaaacaaagcagtgcagtcagtccagagctccataggaaatttgatagtagcaattaaatcggtccaggattatgtcaacaaagaaatcgtgccatcaattgcgagattaggttgtgaagcagcaggacttcagttaggaattgcattaacacagcattactcagaattaacaaacatattcggtgataacataggatcgttacaagaaaaagggataaaattacaaggtatagcatcattataccgcacaaatatcacagagatattcacaacatcaacagttgataaatatgatatttatgatctattatttacagaatcaataaaggtgagagttatagatgttgacttgaatgattactcaatcaccctccaagtcagactccctttattaactagactgctgaacacccagatttacaaagtagatgccatatcatacaacatccaaaacagagaatggtatatccctcttcccagccacatcatgacaaaaggggcatttctaggtggagcagatgtcaaagaatgtatagaagcattcagcagttatatatgcccttctgatccaggatttgtactaaaccatgaaatggagagctgtttatc

SEQ ID NO: 46 - DNA, 1047 bp, humanParainfluenzatype3C243
ttattacaaccatgattatggcatctttctgccaaatagatatcacaaaactacagcatgtaggtgtattggttaacagtcccaaagggatgaagatatcacaaaactttgaaacaagatatctaattttgagcctcataccaaaaatagaagattctaactcttgtggtgaccaacagatcaagcaatacaagaggttattggatagactgatcattcctttatatgatggattaagattacagaaggatgtgatagtgtccaatcaagaatccaatgaaaacactgaccccagaacaaaacgattctttggaggggtaattggaactattgctctgggagtggcaacctcagcacaaattacagcggcagttgctctggttgaagccaagcaggcaagatcagacattgaaaaactcaaggaagcaatcagggacacaaacaaagcagtgcagtcagtccagagctccataggaaatttgatagtagcaattaaatcggtccaggattatgtcaacaaagaaatcgtgccatcaattgcgagattaggttgtgaagcagcaggacttcagttaggaattgcattaacacagcattactcagaattaacaaacatattcggtgataacataggatcgttacaagaaaaagggataaaattacaaggtatagcatcattataccgcacaaatatcacagagatattcacaacatcaacagttgataaatatgatatttatgatctattatttacagaatcaataaaggtgagagttatagatgttgacttgaatgattactcaatcaccctccaagtcagactccctttattaactagactgctgaacacccagatttacaaagtagattccatatcatacaacatccaaaacagagaatggtatatccctcttcccagccacatcatgacaaaaggggcatttctaggtggagcagatgtcaaagaatgtatagaagcattcagcagttatatatgcccttctgatccaggatttgtactaaaccatgaaatggagagctgttatc

SEQ ID NO: 47 - DNA, 1048 bp, humanParainfluenzatype3 CCRI-15706
ttattacaaccatgattatggcatctttctgccaaatagatatcacaaaactacagcatgtaggtgtattggttaacagtcccaaagggatgaagatatcacaaaactttgaaacaagatatctaattttgagcctcataccaaaaatagaagattctaactcttgtggtgaccaacagatcaagcaatacaagaggttattggatagactgatcattcctttatatgatggattaagattacagaaggatgtgatagtgtccaatcaagaatccaatgaaaacactgaccccagaacaaaacgattctttggaggggtaattggaactattgctctgggagtggcaacctcagcacaaattacagcggcagttgctctggttgaagccaagcaggcaagatcagacattgaaaaactcaaggaagcaatcagggacacaaacaaagcagtgcagtcagtccagagctccataggaaatttgatagtagcaattaaatcggtccaggattatgtcaacaaagaaatcgtgccatcaattgcgagattaggttgtgaagcagcaggacttcagttaggaattgcattaacacagcattactcagaattaacaaacatattcggtgataacataggatcgttacaagaaaaagggataaaattacaaggtatagcatcattataccgcacaaatatcacagagatattcacaacatcaacagttgataaatatgatatttatgatctattatttacagaatcaataaaggtgagagttatagatgttgacttgaatgattactcaatcaccctccaagtcagactccctttattaactagactgctgaacacccagatttacaaagtagatgccatatcatacaacatccaaaacagagaatggtatatccctcttcccagccacatcatgacaaaaggggcatttctaggtggagcagatgtcaaagaatgtatagaagcattcagcagttatatatgcccttctgatccaggatttgtactaaaccatgaaatggagagctgtttatc

SEQ ID NO: 48 - DNA, 1054 bp, humanParainfluenzatype3CCRI-15706cloned
ttattacaaccatgattatggcatctttctgccaaatagatatcacaaaactacagcatgtaggtgtattggttaacagtcccaaagggatgaagatatcacaaaactttgaaacaagatatctaattttgagcctcataccaaaaatagaagattctaactcttgtggtgaccaacagatcaagcaatacaagaggttattggatagactgatcattcctttatatgatggattaagattacagaaggatgtgatagtgtccaatcaagaatccaatgaaaacactgaccccagaacaaaacgattctttggaggggtaattggaactattgctctgggagtggcaacctcagcacaaattacagcggcagttgctctggttgaagccaagcaggcaagatcagacattgaaaaactcaaggaagcaatcagggacacaaacaaagcagtgcagtcagtccagagctccataggaaatttgatagtagcaattaaatcggtccaggattatgtcaacaaagaaatcgtgccatcaattgcgagattaggttgtgaagcagcaggacttcagttaggaattgcattaacacagcattactcagaattaacaaacatattcggtaataacataggatcgttacaagaaaaagggataaaattacaaggtatagcatcattataccgcacaaatatcacagagatattcacaacatcaacagttgataaatatgatatttatgatctattatttacagaatcaataaaggtgagagttatagatgttgacttgaatgattactcaatcaccctccaagtcagactccctttattaactagactgctgaacacccagatttacaaagtagatgccatatcatacaacatccaaaacagagaatggtatatccctcttcccagccacatcatgacaaaaggggcatttctaggtggagcagatgtcaaagaatgtatagaagcattcagcagttatatatgcccttctgatccaggatttgtactaaaccatgaaatggagagctgtttatcaggaaa

SEQ ID NO: 49 - DNA, 693 bp, humanCoronavirusOC-43cloned
ggccgcgggaattcgattccagcaccagtttatatctggactgctgatgaagctattaaattcctaaaggaatggaatttttctttgggtattatactactttttattacaatcatattgcaatttggatatacaagtcgcagtatgtttgtttatgttattaagatgattattttgtggcttatgtggccccttactataatcttaactattttcaattgcgtatacgcattgaataatgtgtatcttggcctttctatagtttttaccatagtggccattattatgtggattgtgtattttgtgaatagtatcaggttgtttattagaactggaagtttttggagtttcaacccagaaacaaacaacttgatgtgtatagatatgaaaggaacaatgtatgttaggccgataattgaggactatcatactctgacggtcacaataatacgcggccatctttacattcaaggtataaaactaggtactggctattctttggcagatttgccagcttatatgactgttgctaaggttacacacctgtgcacatataagcgtggttttcttgacaggataagcgatactagtggttttgctgtttatgttaagtccaaagtcggtaattaccgactgccatcaacccaaaagggttctggcatggacaccgcattgttgaatcactagtgaatt

SEQ ID NO: 50 - DNA, 2965 bp, humanCoronavirus229Ecloned
gctcgagcggccgccagtgtgatggatatctgcagaattcgcccttaggcaagtgggtacaagtgcctataggtacaaatgaccctataagattttgtcttgaaaatactgtttgtaaagtttgtggttgttggcttaatcatggctgtacatgtgaccggactgctatccaaagttttgataacagttatttaaacgagtccggggctctagtgccgctcgactagagccctgtaatggtacagacatagattactgtgtccgtgcatttgacgtttacaataaagatgcgtcttttatcggaaaaaatctgaagtccaattgtgtgcgcttcaagaatgtagataaggatgacgcgttctatattgttaaacgttgcattaagtcagttatggaccacgagcagtccatgtataacttacttaaaggctgtaatgctgttgctaagcatgatttctttacttggcatgagggcagaaccatttatggtaatgttagtagacaggatcttactaaatacaccatgatggatttgtgcttcgctctgcgtaactttgatgaaaaagactgtgaagtttttaaggagatattggttcttactggttgttgtagtactgattactttgaaatgaagaattggtttgaccccatagaaaatgaggacatacaccgtgtgtatgctgctttaggtaaggtagttgcaaatgcaatgcttaagtgtgttgctttttgcgacgaaatggtgctcaaaggagttgttggtgttttgaccttagacaaccaagatcttaatgggaatttctatgacttcggtgactttgtattgtgtcctcctggaatgggaataccctactgcacgtcatactattcttatatgatgcctgttatgggtatgactaattgtttagctagtgagtgctttatgaaaagtgacatctttggtcaagacttcaaaacttttgatttgttgaaatatgatttcacagaacataaggaggttttgtttaacaagtactttaagtattggggacaggattatcatcctgattgtgttgattgccatgacgagatgtgtattttgcattgttcaaattttaacacactcttcgcaaccacaattccaaacacggcttttggacctctatgcagaaaagtgtttattgatggtgtacccgtagttgctactgctggttaccactttaaacaattaggacttgtgtggaacaaagatgttaacactcattctaccagacttactattactgaactcttacagtttgtgacagatccaacgcttatagttgcgtcatcgcctgccttggtggataaacgcactgtttgtttttctgtcgctgctttgagtacaggattaacatcccaaacagtaaaacctggccattttaataaggagttttatgacttcttacgttctcaggggtttttcgatgagggttcagaattaacattgaagcatttcttttttacacaaaagggtgatgctgcaattaaagattttgattattatcgttacaacagacctactatgctggatattggacaagctcgcgtagcatatcaagtggcagctcgctattttgactgttacgagggtggctgtattacatctagagaggttgttgttacaaaccttaataaaagcgctggttggccccttaataagtttggtaaagctggtttatattatgagtctattagttatgaggaacaagatgctattttttcattaacaaagcgtaatattctccctactatgactcagttaaatcttaaatacgccatatctggtaaggaacgcgcacgtacagtgggtggcgtctctttattagctactatgactacaagacagtttcatcagaaatgtctgaaatccatagtagctaccagaaatgccaccgttgttatcggcactaccaagttttatggcgggtgggataatatgttaaagaacctgatggccgatgttgatgatcctaaattgatgggatgggactatcctaagtgtgatagagctatgccctcaatgattcgtatgttgtcggctatgatcttaggttctaagcatgtcacatgttgtacggctagtgataaattttatagacttagtaatgagcttgctcaagttttgaccgaggttgtttattcaaatggtgggttttattttaaacctggtggtacaacttctggtgatgcaactacagcctatgccaattctgtctttaatatatttcaggctgtaagttctaacattaattgcgttttgagcgttaactcgtcaaattgcaataattttaatgttaagaagttacagagacaactttatgataattgctatagaaatagtaatgttgatgaatcttttgtggatgacttttatggttatttgcaaaagcatttttctatgatgattctttctgatgatagtgttgtgtgctataataaaacttatgctggacttggttacattgctgatattagtgcttttaaagccactttgtattatcagaatggtgtgtttatgagtacagctaagtgttggactgaggaagatctttctataggacctcatgaattttgctcacagcacactatgcagattgtagatgaaaatggtaagtattatctaccatatccagatcctagccgtattatttctgctggtgtttttgtggatgacatcactaagactgatgctgtcattcttttggaacgctatgtttctctggctatagatgcctacccattgtctaagcatcctaaacctgagtacaggaaggtgttttacgcattgttagactgggtcaaacatctcaacaagactcttaacgaaggtgttttggagtctttttctgttacacttttagatgaacatgagtctaagttttgggatgaaagcttttatgctagtatgtatgagaagtctacagtattacaagctgctggtctt

SEQ ID NO: 51 - DNA, 1752 bp, humanCoronavirusNL CCRI-16014cloned
tgcagcattgagtactggtttgacaaatcaagttgttaagccaggtcattttaatgaagagttttataactttcttcgtttaagaggtttctttgatgaaggttctgaacttacattaaaacatttcttcttcgcacagaatggtgatgctgctgttaaagattttgacttttaccgttataataagcctaccattttagatatttgtcaagctagagttacatataagatagtctctcgttattttgacatttatgaaggtggctgtattaaggcatgtgaagttgttgtaacaaatcttaataagagtgctggttggccattaaataagtttggtaaagctagtttgtattatgaatctatatcttatgaagaacaggatgctttgtttgctttgacaaagcgtaatgtcctccctactatgacacagctgaatcttaagtatgctattagtggtaaagaacgtgctagaactgttggtggtgtttctctgttgtctacaatgaccacaagacaataccatcaaaaacatcttaaatccattgttaatacacgcaatgccactgttgttattggtactaccaaattttatggtggttggaataatatgttgcgtactttaattgatggtgttgaaaaccctatgctcatgggttgggattatcccaaatgtgatagagctttgcctaacatgatacgtatgatttcagccatggtgttgggctctaagcatgttaattgttgtactgcaacagataggttttataggcttggtaatgagttggcacaagttttaacagaagttgtttattctaatggtggtttttattttaagccaggtggtacgacttctggtgacgctagtacagcttatgctaattctatttttaacatttttcaagccgtgagttctaacattaacaggttgcttagtgtcccatcagattcatgtaataatgttaatgttagggatttacaacgacgtctgtatgataattgctataggttaactagtgtcgaagagtcattcattgatgattattatggttatcttaggaaacatttttcaatgatgattctctctgatgacggtgttgtctgttataacaaggattatgctgagttaggttatatagcagacattagtgcttttaaagccactttgtattaccagaataatgtttttatgagtacttctaaatgttgggttgaagaagatttaactaagggaccacatgagttttgttcccagcatactatgcaaatagttgacaaagatggtacctattatttgccttacccagatcctagtaggatcttgtcagctggtgtttttgttgatgatgttgttaagacagatgctgttgttttgttagaacgttatgtgtctttagctattgatgcataccctctttcaaaacaccctaattctgaatatcgtaaggttttttacgtattacttgattgggttaagcatcttaacaaaaatttgaatgagggtgttcttgaatctttttctgttacacttcttgataatcaagaagataagttttggcgtgaagatttttatgctagtatgtatgaaaattctacaatattgcaagctgctggtttatgtgttgtttgtggttcacaaactgtacttcgttgtggtgattgtctgcgtaagcctatgttgtgcactaaatgcgcatatgatcatgtatttggtaccgaccacaagtttattttggctataacaccgtatgtatg

SEQ ID NO: 52 - DNA, 1703 bp, humanCoronavirusSARScloned
atttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagtt

SEQ ID NO: 53 - DNA, 2104 bp, humanAdenovirus CCRI-15636
tttagaaaccccacagtggcgcccacccacgatgtgaccaccgatcgtagccagcgcctgatgctgcgcttcgtgcccgttgaccgggaagacaatacctactcttacaaagttcgctacacgctggctgtaggcgacaacagagtgcttgacatggccagcacattctttgacattcggggggtgcttgatagaggtcctagcttcaagccatattccggcacagcttacaattcactcgctcctaagggcgcgcccaatacatctcagtggatagttacaacgaatcgagacaatgcagtaactaccaccacaaacacatttggcattgcttccatgaagggagacaatattactaaagaaggtttgcaaattgggaaagacattaccactactgaaggagaagaaaagcccatttatgccgataaaacatatcagccagagcctcaagttggagaagaatcatggactgatactgatggaacaaatgaaaagtttggtggaagagcccttaaaccagctaccaacatgaagccatgctacgggtcttttgcaagacctacaaacataaaagggggccaagctaaaaacagaaaagtaaaaccaacaaccgaaggaggggttgaaactgaggaaccagatattgatatggaatttttcgatggtagagatgctgttgcaggagctttagcgcctgaaattgtgctttatacggaaaatgtaaatttggaaactccagacagtcatgtggtatataaaccaggaacgtctgataactctcatgcaaatttgggtcaacaagccatgcctaacagacccaattacattggattcagggataactttgtaggcctaatgtactacaacagtactggaaatatgggagttttggctggccaagcatcacaactgaatgcagtggttgacttgcaggacagaaatactgaactgtcatatcagcttttgcttgattctctgggagacagaaccagatacttcagcatgtggaatcaggctgtggacagttacgatcccgatgttcgcattattgaaaatcatggcatcgaggatgaactgcctaattactgttttcctctggatggcataggaccagggcacaggtatcaaggcattaaagttaaaaccgatgacgctaatggatgggaaaaagatgctaatgttgatacagctaatgaaatagccataggaaacaacctggctatggaaattaatatccaagctaacctttggagaagttttctgtactccaatgtggctttgtaccttccagatgtttacaagtacacgccacctaacattactttgcccactaacaccaacacctatgagtacatgaacgggcgagtggtatccccatctctggttgattcatacatcaacatcggcgccaggtggtctcttgacccaatggacaatgtgaatccattcaaccaccaccgcaatgctggtctgcgctacaggtccatgcttctgggaaatggtcgttatgtgcctttccacatacaagtgcctcaaaaattctttgctgtcaaaaacctacttcttctacctggctcctacacctacgagtggaacttcagaaaggatgtgaacatggtcctgcaaagttcccttggaaatgacctcagaacagatggtgctaccataagtttcaccagcatcaatctctatgccaccttcttccccatggctcacaacacagcttccacccttgaagccatgctgcgcaacgataccaatgatcagtcatttaacgactacctctctgcagctaacatgctttaccccattcctgccaatgcaaccaacattccaatttccatcccatctcgcaactgggcagccttcaggggctggtccttcaccaggctcaaaaccaaggagactccatctcttggatcagggttcgatccctacttcgtatattctggatctattccctacctggatggcaccttctaccttaaccacactttcaagaaggtctccatcatgtttgactcctcagtcagctggcctggcaatgacaggctgttgagcccaaatgagtttgaaatcaagcgcactgtggacggggaaggat

SEQ ID NO: 54 - DNA, 2116 bp, humanAdenovirus CCRI-15625
atggggacaaatttagaaaccccacagtggcgcccacccacgatgtgaccaccgatcgtagccagcgcctgatgctgcgcttcgtgcccgttgaccgggaagacaatacctactcttacaaagttcgctacacgctggctgtaggcgacaacagagtgcttgacatggccagcacattctttgacattcggggggtgcttgatagaggtcctagcttcaagccatattccggcacagcttacaattcactcgctcctaagggcgcgcccaatacatctcagtggatagttacaacgaatcgagacaatgcagtaactaccaccacaaacacatttggcattgcttccatgaagggagacaatattactaaagaaggtttgcaaattgggaaagacattaccactactgaaggagaagaaaagcccatttatgccgataaaacatatcagccagagcctcaagttggagaagaatcatggactgatactgatggaacaaatgaaaagtttggtggaagagcccttaaaccagctaccaacatgaagccatgctacgggtcttttgcaagacctacaaacataaaagggggccaagctaaaaacagaaaagtaaaaccaacaaccgaaggaggggttgaaactgaggaaccagatattgatatggaatttttcgatggtagagatgctgttgcaggagctttagcgcctgaaattgtgctttatacggaaaatgtaaatttggaaactccagacagtcatgtggtatataaaccaggaacgtctgataactctcatgcaaatttgggtcaacaagccatgcctaacagacccaattacattggattcagggataactttgtaggcctaatgtactacaacagtactggaaatatgggagttttggctggccaagcatcacaactgaatgcagtggttgacttgcaggacagaaatactgaactgtcatatcagcttttgcttgattctctgggagacagaaccagatacttcagcatgtggaatcaggctgtggacagttacgatcccgatgttcgcattattgaaaatcatggcatcgaggatgaactgcctaattactgttttcctctggatggcataggaccagggcacaggtatcaaggcattaaagttaaaaccgatgacgctaatggatgggaaaaagatgctaatgttgatacagctaatgaaatagccataggaaacaacctggctatggaaattaatatccaagctaacctttggagaagttttctgtactccaatgtggctttgtaccttccagatgtttacaagtacacgccacctaacattactttgcccactaacaccaacacctatgagtacatgaacgggcgagtggtatccccatctctggttgattcatacatcaacatcggcgccaggtggtctcttgacccaatggacaatgtgaatccattcaaccaccaccgcaatgctggtctgcgctacaggtccatgcttctgggaaatggtcgttatgtgcctttccacatacaagtgcctcaaaaattctttgctgtcaaaaacctacttcttctacctggctcctacacctacgagtggaacttcagaaaggatgtgaacatggtcctgcaaagttcccttggaaatgacctcagaacagatggtgctaccataagtttcaccagcatcaatctctatgccaccttcttccccatggctcacaacacagcttccacccttgaagccatgctgcgcaacgataccaatgatcagtcatttaacgactacctctctgcagctaacatgctttaccccattcctgccaatgcaaccaacattccaatttccatcccatctcgcaactgggcagccttcaggggctggtccttcaccaggctcaaaaccaaggagactccatctcttggatcagggttcgatccctacttcgtatattctggatctattccctacctggatggcaccttctaccttaaccacactttcaagaaggtctccatcatgtttgactcctcagtcagctggcctggcaatgacaggctgttgagcccaaatgagtttgaaatcaagcgcactgtggacggggaaggata

SEQ ID NO: 55 - DNA, 2102 bp, humanAdenovirus CCRI-15942
tttagaaaccccacagtggcgcccacccacgatgtgaccaccgatcgtagccagcgcctgatgctgcgcttcgtgcccgttgaccgggaagacaatacctactcttacaaagttcgctacacgctggctgtaggcgacaacagagtgcttgacatggccagcacattctttgacattcggggggtgcttgatagaggtcctagcttcaagccatattccggcacagcttacaattcactcgctcctaagggcgcgcccaatacatctcagtggatagttacaacgaatcgagacaatgcagtaactaccaccacaaacacatttggcattgcttccatgaagggagacaatattactaaagaaggtttgcaaattgggaaagacattaccactactgaaggagaagaaaagcccatttatgccgataaaacatatcagccagagcctcaagttggagaagaatcatggactgatactgatggaacaaatgaaaagtttggtggaagagcccttaaaccagctaccaacatgaagccatgctacgggtcttttgcaagacctacaaacataaaagggggccaagctaaaaacagaaaagtaaaaccaacaaccgaaggaggggttgaaactgaggaaccagatattgatatggaatttttcgatggtagagatgctgttgcaggagctttagcgcctgaaattgtgctttatacggaaaatgtaaatttggaaactccagacagtcatgtggtatataaaccaggaacgtctgataactctcatgcaaatttgggtcaacaagccatgcctaacagacccaattacattggattcagggataactttgtaggcctaatgtactacaacagtactggaaatatgggagttttggctggccaagcatcacaactgaatgcagtggttgacttgcaggacagaaatactgaactgtcatatcagcttttgcttgattctctgggagacagaaccagatacttcagcatgtggaatcaggctgtggacagttacgatcccgatgttcgcattattgaaaatcatggcatcgaggatgaactgcctaattactgttttcctctggatggcataggaccagggcacaggtatcaaggcattaaagttaaaaccgatgacgctaatggatgggaaaaagatgctaatgttgatacagctaatgaaatagccataggaaacaacctggctatggaaattaatatccaagctaacctttggagaagttttctgtactccaatgtggctttgtaccttccagatgtttacaagtacacgccacctaacattactttgcccactaacaccaacacctatgagtacatgaacgggcgagtggtatccccatctctggttgattcatacatcaacatcggcgccaggtggtctcttgacccaatggacaatgtgaatccattcaaccaccaccgcaatgctggtctgcgctacaggtccatgcttctgggaaatggtcgttatgtgcctttccacatacaagtgcctcaaaaattctttgctgtcaaaaacctacttcttctacctggctcctacacctacgagtggaacttcagaaaggatgtgaacatggtcctgcaaagttcccttggaaatgacctcagaacagatggtgctaccataagtttcaccagcatcaatctctatgccaccttcttccccatggctcacaacacagcttccacccttgaagccatgctgcgcaacgataccaatgatcagtcatttaacgactacctctctgcagctaacatgctttaccccattcctgccaatgcaaccaacattccaatttccatcccatctcgcaactgggcagccttcaggggctggtccttcaccaggctcaaaaccaaggagactccatctcttggatcagggttcgatccctacttcgtatattctggatctattccctacctggatggcaccttctaccttaaccacactttcaagaaggtctccatcatgtttgactcctcagtcagctggcctggcaatgacaggctgttgagcccaaatgagtttgaaatcaagcgcactgtggacggggaagg

SEQ ID NO: 56 - DNA, 2115 bp, humanAdenovirus CCRI-15517
gtatggggacaatttagaaaccccacagtggcgcccacccacgatgtgaccaccgatcgtagccagcgcctgatgctgcgcttcgtgcccgttgaccgggaagacaatacctactcttacaaagttcgctacacgctggctgtaggcgacaacagagtgcttgacatggccagcacattctttgacattcggggggtgcttgatagaggtcctagcttcaagccatattccggcacagcttacaattcactcgctcctaagggcgcgcccaatacatctcagtggatagttacaacgaatcgagacaatgcagtaactaccaccacaaacacatttggcattgcttccatgaagggagacaatattactaaagaaggtttgcaaattgggaaagacattaccactactgaaggagaagaaaagcccatttatgccgataaaacatatcagccagagcctcaagttggagaagaatcatggactgatactgatggaacaaatgaaaagtttggtggaagagcccttaaaccagctaccaacatgaagccatgctacgggtcttttgcaagacctacaaacataaaagggggccaagctaaaaacagaaaagtaaaaccaacaaccgaaggaggggttgaaactgaggaaccagatattgatatggaatttttcgatggtagagatgctgttgcaggagctttagcgcctgaaattgtgctttatacggaaaatgtaaatttggaaactccagacagtcatgtggtatataaaccaggaacgtctgataactctcatgcaaatttgggtcaacaagccatgcctaacagacccaattacattggattcagggataactttgtaggcctaatgtactacaacagtactggaaatatgggagttttggctggccaagcatcacaactgaatgcagtggttgacttgcaggacagaaatactgaactgtcatatcagcttttgcttgattctctgggagacagaaccagatacttcagcatgtggaatcaggctgtggacagttacgatcccgatgttcgcattattgaaaatcatggcatcgaggatgaactgcctaattactgttttcctctggatggcataggaccagggcacaggtatcaaggcattaaagttaaaaccgatgacgctaatggatgggaaaaagatgctaatgttgatacagctaatgaaatagccataggaaacaacctggctatggaaattaatatccaagctaacctttggagaagttttctgtactccaatgtggctttgtaccttccagatgtttacaagtacacgccacctaacattactttgcccactaacaccaacacctatgagtacatgaacgggcgagtggtatccccatctctggttgattcatacatcaacatcggcgccaggtggtctcttgacccaatggacaatgtgaatccattcaaccaccaccgcaatgctggtctgcgctacaggtccatgcttctgggaaatggtcgttatgtgcctttccacatacaagtgcctcaaaaattctttgctgtcaaaaacctacttcttctacctggctcctacacctacgagtggaacttcagaaaggatgtgaacatggtcctgcaaagttcccttggaaatgacctcagaacagatggtgctaccataagtttcacctgcatcaatctctatgccaccttcttccccatggctcacaacacagcttccacccttgaagccatgctgcgcaacgataccaatgatcagtcatttaacgactacctctctgcagctaacatgctttaccccattcctgccaatgcaaccaacattccaatttccatcccatctcgcaactgggcagccttcaggggctggtccttcaccaggctcaaaaccaaggagactccatctcttggatcagggttcgatccctacttcgtatattctggatctattccctacctggatggcaccttctaccttaaccacactttcaagaaggtctccatcatgtttgactcctcagtcagctggcctggcaatgacaggctgttgagcccaaatgagtttgaaatcaagcgcactgtggacggggaagga

SEQ ID NO: 57 - DNA, 2173 bp, humanAdenovirusCCRI-15517cloned
cgcccgtgcaacagacacctacttcagtatggggaacaaatttagaaaccccacagtggcgcccacccacgatgtgaccaccgatcgtagccagcgcctgatgctgcgcttcgtgcccgttgaccgggaagacaatacctactcttacaaagttcgctacacgctggctgtaggcgacaacagagtgcttgacatggccagcacattctttgacattcggggggtgcttgatagaggtcctagcttcaagccatattccggcacagcttacaattcactcgctcctaagggcgcgcccaatacatctcagtggatagttacaacgaatcgagacaatgcagtaactaccaccacaaacacatttggcattgcttccatgaagggagacaatattactaaagaaggtttgcaaattgggaaagacattaccactactgaaggagaagaaaagcccatttatgccgataaaacatatcagccagagcctcaagttggagaagaatcatggactgatactgatggaacaaatgaaaagtttggtggaagagcccttaaaccagctaccaacatgaagccatgctacgggtcttttgcaagacctacaaacataaaagggggccaagctaaaaacagaaaagtaaaaccaacaaccgaaggaggggttgaaactgaggaaccagatattgatatggaatttttcgatggtagagatgctgttgcaggagctttagcgcctgaaattgtgctttatacggaaaatgtaaatttggaaactccagacagtcatgtggtatataaaccaggaacgtctgataactctcatgcaaatttgggtcaacaagccatgcctaacagacccaattacattggattcagggataactttgtaggcctaatgtactacaacagtactggaaatatgggagttttggctggccaagcatcacaactgaatgcagtggttgacttgcaggacagaaatactgaactgtcatatcagcttttgcttgattctctgggagacagaaccagatacttcagcatgtggaatcaggctgtggacagttacgatcccgatgttcgcattatggaaaatcatggcatcgaggatgaactgcctaattactgttttcctctggatggcataggaccagggcacaggtatcaaggcattaaagttaaaaccgatgacgctaatggatgggaaaaagatgctaatgttgatacagctaatgaaatagccataggaaacaacctggctatggaaattaatatccaagctaacctttggagaagttttctgtactccaatgtggctttgtaccttccagatgtttacaagtacacgccacctaacattactttgcccactaacaccaacacctatgagtacatgaacgggcgagtggtatccccatctctggttgattcatacatcaacatcggcgccaggtggtctcttgacccaatggacaatgtgaatccattcaaccaccaccgcaatgctggtctgcgctacaggtccatgcttctgggaaatggtcgttatgtgcctttccacatacaagtgcctcaaaaattctttgctgtcaaaaacctacttcttctacctggctcctacacctacgagtggaacttcagaaaggatgtgaacatggtcctgcaaagttcccttggaaatgacctcagaacagatggtgctaccataagtttcaccagcatcaatctctatgccaccttcttccccatggctcacaacacagcttccacccttgaagccatgctgcgcaacgataccaatgatcagtcatttaacgactacctctctgcagctaacatgctttaccccattcctgccaatgcaaccaacattccaatttccatcccatctcgcaactgggcagccttcaggggctggtccttcaccaggctcaaaaccaaggagactccatctcttggatcagggttcgatccctacttcgtatattctggatctattccctacctggatggcaccttctaccttaaccacactttcaagaaggtctccatcatgtttgactcctcagtcagctggcctggcaatgacaggctgttgagcccaaatgagtttgaaatcaagcgcactgtggacggggaaggatacaacgtggcacaatgcaacatgaccaaa

SEQ ID NO: 58 - DNA, 2166 bp, humanAdenovirusCCRI-15673
gtttagaaaccccacggtggcgcctacgcacgacgtgaccacagaccggtcccagcgtttgacgctgcggttcatccctgtggatcgcgaggataccgcgtactcgtacaaggcgcggttcaccctggctgtgggtgacaaccgcgtgcttgatatggcttccacgtactttgacatccgcggcgtgctggacaggggccctactttcaagccctactctggcactgcctacaacgctctagctcccaagggcgctcccaactcctgtgagtgggaacaaaccgaagatagcggccgggcggttgccgaggatgaagaagaagaggaagatgaagatgaagaagaggaagaggaagagcaaaacgctcgagatcaggctactaagaaaacacatgtttatgcccaggctcctttgtctggagaaacaattacaaaaagcgggctacaaataggatcagacaatgcagaaacacaagctaaacctgtatatgcagatccttcctatcaaccagaacctcaaattggcgaatctcagtggaacgaagctgatgctaatgcagcaggagggagagtgcttaaaaaaacaactcccatgaaaccatgctatggatcttatgccaggcctacaaatccttttggtggccaatccgtcctggttccggatgaaaaaggggtgcctcttccaaaggttgacttgcaattcttctcaaatactacctctttgaacgaccggcaaggcaatgccactaaaccaaaagtggtgttgtacagtgaagatgtaaatttggaaaccccagacacacatctgtcttacaaacctggaaaaggtgatgaaaattctaaagctatgttgggtcaacaatctatgccaaacagacccaattacatcgctttcagggacaattttattggcctaatgtattataacagcactggcaacatgggtgttcttgctggtcaggcatcgcagctaaatgccgtggtagatttgcaagacagaaacacagagctgtcctatcaacttttgcttgattccataggtgatagaaccagatatttttctatgtggaatcaggctgtagacagctatgacccagatgttagaatcattgaaaaccatggaactgaggatgaattgccaaattattgtttccctcttgggggtattggggtaactgacacctatcaagctattaaggctaatggcaatggcgcaggtgataatggaaatactacatggacaaaagatgaaacttttgcaacacgtaatgaaataggagtgggtaacaactttgccatggaaattaacctaaatgccaacctatggagaaatttcctttactccaatattgcactatacctgccagacaagctaaaatacaaccccaccaacgtggaaatatctgacaaccccaacacctacgactacatgaacaagcgagtggtggctcctgggcttgtagactgctacattaaccttggggcgcgctggtctctggactacatggacaacgttaatccctttaaccaccaccgcaatgcgggcctgcgttaccgctccatgttgttgggaaacggccgctacgtgccttttcacattcaggtgccccaaaagttttttgccattaaaaacctcctcctcctgccaggctcatacacatatgaatggaacttcaggaaggatgttaacatggttctgcagagctctctgggaaacgaccttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctatatcccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacttttgactcttctgttagctggccgggcaacgaccgcctgcttactcccaatgagtttgagattaagcgctcagt

SEQ ID NO: 59 - DNA, 693 bp, humanInfluenzaB CCRI-15829
aattgcctacctgctttcattgacagaagatggagaaggcaaagcagaactagcagaaaaattacactgttggttcggtgggaaagaatttgacctagactctgccttggaatggataaaaaacaaaagatgcttaactgatatacagaaagcactaattggtgcctctatctgctttttaaaacccaaagaccaggaaagaaaaagaagattcatcacagagcccctatcaggaatgggaacaacagcaacaaaaaagaagggcctgattctagctgagagaaaaatgagaaaatgtgtgagcttccatgaagcatttgaaatagcagaaggccatgaaagctcagcgttactatattgtctcatggtcatgtacctgaatcctggaaattattcaatgcaagtaaaactaggaacgctctgtgctttgtgcgaaaaacaagcatcacattcacacagggctcatagcagagcagcgagatcttcagtgcccggagtgagacgagaaatgcagatggtctcagctatgaacacagcaaaaacaatgaatggaatgggaaaaggagaagacgtccaaaaactggcagaagaactgcaaagcaacattggagtattgagatctcttggggcaagtcaaaagaatggggaaggaattgcaaaggatgtaatggaagtgctaaagcagagctctat

SEQ ID NO: 60 - DNA, 667 bp, humanInfluenzaB CCRI-15750
aagatggagaaggcaaagcagaactagcagaaaaattacactgttggttcggtgggaaagaatttgacctagactctgccttggaatggataaaaaacaaaagatgcttaactgatatacagaaagcactaattggtgcctctatctgctttttaaaacccaaagaccaggaaagaaaaagaagattcatcacagagcccctatcaggaatgggaacaacagcaacaaaaaagaagggcctgattctagctgagagaaaaatgagaaaatgtgtgagcttccatgaagcatttgaaatagcagaaggccatgaaagctcagcgttactatattgtctcatggtcatgtacctgaatcctggaaattattcaatgcaagtaaaactaggaacgctctgtgctttgtgcgaaaaacaagcatcacattcacacagggctcatagcagagcagcgagatcttcagtgcccggagtgagacgagaaatgcagatggtctcagctatgaacacagcaaaaacaatgaatggaatgggaaaaggagaagacgtccaaaaactggcagaagaactgcaaagcaacattggagtattgagatctcttggggcaagtcaaaagaatggggaaggaattgcaaaggatgtaatggaagtgctaaagcagagctctat

SEQ ID NO: 61 - DNA, 694 bp, humanInfluenzaB CCRI-15803
caattgcctacctgctttcattgacagaagatggagaaggcaaagcagaactagcagaaaaattacactgttggttcggtgggaaagaatttgacctagactctgccttggaatggataaagaacaaaagatgcttaactgatatacagaaagcactaattggtgcctctatctgctttttaaaacccaaagaccaggaaagaaaaagaagattcatcacagagcccctatcaggaatgggaacaacagcaacaaaaaagaagggcctgattctagctgagagaaaaatgagaaaatgtgtgagcttccatgaagcatttgaaatagcagaaggccatgaaagctcagcgttactatattgtctcatggtcatgtacctgaatcctggaaattattcaatgcaagtaaaactaggaacgctctgtgctttgtgcgaaaaacaagcatcacattcacacagggctcatagcagagcagcgagatcttcagtgcccggagtgagacgagaaatgcagatggtctcagctatgaacacagcaaaaacaatgaatggaatgggaaaaggagaagacgtccaaaaactggcagaagaactgcaaagcaacattggagtattgagatctcttggggcaagtcaaaagaatggggaaggaattgcaaaggatgtaatggaagtgctaaagcagagctctat

SEQ ID NO: 62 - DNA, 693 bp, humanInfluenzaB CCRI-15751
aattgcctacctgctttcattgacagaagatggagaaggcaaagcagaactagcagaaaaattacactgttggtttggtgggaaagaatttgacctagactctgccttggaatggataaaaaacaaaagatgcttaactgatatacaaaaagcactaattggtgcctctatctgctttttaaaacccaaagaccaggaaagaaaaagaagattcatcacagagcccttatcaggaatgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttgtgcgagaaacaagcatcacattcacacagggctcatagcagagcagcgagatcttcagtgcctggagtgagacgagaaatgcagatggtctcagctatgaacacagcaaaaacaatgaatggaatgggaaaaggagaagacgtccaaaagctggcagaagagctgcaaagcaacattggagtgctgagatctcttggggcaagtcaaaagaatggggaaggaattgcaaaggatgtaatggaagtgctaaagcagagctctat

SEQ ID NO: 63 - DNA, 693 bp, humanInfluenzaB CCRI-15804
aattgcctacctgctttcattgacagaagatggagaaggcaaagcagaactagcagaaaaattacactgttggtttggtgggaaagaatttgacctagactctgccttggaatggataaaaaacaaaagatgcttaactgatatacaaaaagcactaattggtgcctctatctgctttttaaaacccaaagaccaggaaagaaaaagaagattcatcacagagcccttatcaggaatgggaacaacagcaacaaaaaagaaaggcctgattctggctgagagaaaaatgagaagatgtgtgagttttcatgaagcatttgaaatagcagaaggccatgaaagctcagcgctactatactgtctcatggtcatgtacctgaatcctggaaattattcaatgcaagtaaaactaggaacgctctgtgctttgtgcgagaaacaagcatcacattcacacagggctcatagcagagcagcgagatcttcagtgcctggagtgagacgagaaatgcagatggtctcagctatgaacacagcaaaaacaatgaatggaatgggaaaaggagaagacgtccaaaagctggcagaagagctgcaaagcaacattggagtgctgagatctcttggggcaagtcaaaagaatggggaaggaattgcaaaggatgtaatggaagtgctaaagcagagctctat

SEQ ID NO: 64 - DNA, 693 bp, humanInfluenzaBvaccinestrainB/Harbin/07/94
aattgcctacctgctttcattgacagaagatggagaaggcaaagcagaactagcagaaaaattacactgttggttcggtgggaaagaatttgacctagactctgccttggaatggataaaaaacaaaagatgcttaactgatatacaaaaagcactaattggtgcatctatctgctttttaaaacccaaagaccaggaaagaaaaagaagattcatcacagagcccctatcaggaatgggaacaacagcaacaaaaaagaaaggcctgattctagctgagagaaaaatgagaagatgtgtgagctttcatgaagcatttgaaatagcagaaggccatgaaagctcagcgctactatattgtctcatggtcatgtacctgaatcctggaaattattcaatgcaagtaaaactaggaacgctctgtgctttgtgcgagaaacaagcatcacattcacacagggctcatagcagagcagcgagatcttcagtgcccggagtgagacgagaaatgcagatggtctcagctatgaacacagcaaaaacaatgaatggaatgggaaaaggagaagacgtccaaaaactggcagaagagctgcaaagcaacattggagtattgagatctcttggggcaagtcaaaagaatggggaaggaattgcaaaggatgtaatggaagtgctaaagcagagctctat

SEQ ID NO: 65 - DNA, 693 bp, humanInfluenzaBvaccinestrainB/Taiwan/2/62
aattgcctacctgctttcactaacagaagatggagaaggcaaagcagaactagcggaaaaattacactgttggttcggtgggaaagaatttgacctggactctgctttggaatggataaaaaacaaaagatgcctaactgatatacaaaaagcactaattggtgcctctatatgctttttaaaacccaaagaccaagaaagaaaaagaagattcattacagagccactgtcaggaatgggaacaacagcaacaaagaagaaaggcctaattctagctgagagaaaaatgagaagatgtgtaagctttcatgaagcatttgaaatagcagaaggccacgaaagctcagcattactatattgtcttatggtcatgtatctgaatcctggaaactattcaatgcaagtaaaactaggaacgctctgtgctttgtgcgagaaacaagcatcacattcacatagagcccatagcagagcggcaagatcttcagtgcctggagtgaggcgagaaatgcaaatggtttcagctatgaacacagcaaaaacaatgaatggaatggggaagggagaagacgtccaaaaattggcagaagagttgcaaagcaacattggagtgttgagatctctgggagcaagtcaaaagaatggagaaggaattgccaaagatgtaatggaagtgctaaaacagagctctat

SEQ ID NO: 66 - DNA, 694 bp, humanInfluenzaB/Lee/40
caattgcctacctgctttcactaatagaagatggagaaggcaaagcagaactagctgaaaaattacactgttggttcggtgggaaagaatttgacctagattctgctttggaatggataaaaaacaaaaggtgcctaactgatatacaaaaagcactaattggtgcctctatatgctttttaaaacccaaagaccaagaaagaaaaaggagattcatcacagagcccctgtcaggaatgggaacaacagcaacaaagaagaaaggcctaattctagctgagagaaaaatgagaagatgtgtaagctttcatgaagcatttgaaatagcagaaggccacgaaagctcagcattactatattgtcttatggtcatgtacctaaaccctgaaaactattcaatgcaagtaaaactaggaacgctctgtgctttatgcgagaaacaagcatcgcactcgcatagagcccatagcagagcagcaaggtcttcggtacctggagtaagacgagaaatgcagatggtttcagctatgaacacagcaaagacaatgaatggaatgggaaagggagaagacgtccaaagactagcagaagagctgcaaaacaacattggagtgttgagatctctaggagcaagtcaaaagaatggagaaggaattgccaaagatgtaatggaagtgctaaaacagagctccat

SEQ ID NO: 67 - DNA, 735 bp, humanInfluenzaBB/Harbin/07/94cloned
atgtcgctgtttggagacacaattgcctacctgctttcattgacagaagatggagaaggcaaagcagaactagcagaaaaattacactgttggttcggtgggaaagaatttgacctagactctgccttggaatggataaaaaacaaaagatgcttaactgatatacaaaaagcactaattggtgcatctatctgctttttaaaacccaaagaccaggaaagaaaaagaagattcatcacagagcccctatcaggaatgggaacaacagcaacaaaaaagaaaggcctgattctagctgagagaaaaatgagaagatgtgtgagctttcatgaagcatttgaaatagcagaaggccatgaaagctcagcgctactatattgtctcatggtcatgtacctgaatcctggaaattattcaatgcaagtaaaactaggaacgctctgtgctctgtgcgagaaacaagcatcacattcacacagggctcatagcagagcagcgagatcttcagtgcccggagtgagacgagaaatgcagatggtctcagctatgaacacagcaaaaacaatgaatggaatgggaaaaggagaagacgtccaaaaactggcagaagagctgcaaagcaacattggagtattgagatctcttggggcaagtcaaaagaatggggaaggaattgcaaaggatgtaatggaagtgctaaagcagagctctatgggaaattcagctctcgtgaag

SEQ ID NO: 68 - DNA, 1323 bp, humanParainfluenzatype4 CCRI-15568
atctaatgaatctaggaacggtaccaaccgcgattagatctctagtttattacacctacagtaaaccatcttatctcaccgtagatttaattcccaatttgaagaatctagacccaaaatgcaattactcaagtttaaattactataataaaacggcgttaagcttgattcaaccaattgcggacaatatcaatcgccttacaaagccaatcacaagctcagaaattcaaagtcgcttctttggggcagttataggtacagttgctctcggtgtagctactgccgcacaagttacagcagcaatcggtcttgcaaaagctcaggaaaatgcacggcttatactaactctcaaaaaagctgcaaaggaaacaaataatgcagttcgggatcttataaaatccaacaaaattgtggcaaggatgatatcagcaattcagaatcagataaacaccgtcattcaacctgccatagaccggatcaattgtcaaatcaaagacctgcaagttggtaacattttaaatttgtacctgacagagatcacaactgttttccacaatcaattgaccaatcctgcattagaatcaattagtattcaggctcttaaaagtcttctaggatctaccttaccagaagtgctgtctaagttagatctaaacaatatctcggcagcttcagtaatggcatctggcttaattaaaggacagataattgcagttgatataccgacaatgacattagtattgatggtccaaataccaagtatctctcctctaagacaagcaaaaataatagatctaacctctataacaattcacacaaataatcaggaagtacaagctgtggtaccagatagagttcttgagattggttcagagatattaggatttgacggttcagtgtgccaaatcacgaaagatacagtcttttgtccctacaatgatgcatatatattacccatccagcagaaaagatgcctacaaggtcaaacaagagattgcgtgttcaccccagttgctggcactttccctaggagatttctcactacatatggtactatagtagccaattgtagaaatttagtatgttcttgtctacgacctccccaaataatctaccaacctgatgagaatccagttacaatcatagataaagacttatgtacaacattgactctagactccataactatagagatccagaaatccataaatagtacttttcgacgtgaagtagttctagaatctactcaggtcagatctttgactcctcttgatttatcgaccgacttaaatcagtataatcaattactcaagagtgctgaag

SEQ ID NO: 69 - DNA, 1372 bp, humanParainfluenzatype4 CCRI-15512
ttaccaatctggatataactcatttaatgaatctaggaacggtaccaactgcaattagatctctagtttactacacttatactaaaccatcttatctcactgtagacttgattcccaacttgaagaatctggaccaaaattgcaattactcaagcttaaactactacaacaaaactgcactaagcctaattcaaccaattgcagataacatcaatcgccttacaaagcccatcacaagctcagaagttcaaagtcgtttctttggggcagtcataggcacaattgctcttggtgtagccaccgctgcacaagttacagcagcaatcggtctagcaaaagctcaagaaaatgcaaaacttatactaactctcaagaaagctgcaacagaaacaaatgaggcagttcgagatcttgcaaactctaataaaattgttgtgaaaatgatatcagcaattcaaaatcaaataaacactattattcaacctgctatagaccagattaattgtcaaattaaagacctacaggttgccaatatcttaaatttgtacctaacagagataacgactgttttccacaaccaattaaccaatcccgcattagagtcaattagcattcaagctctcaaaagcctcttagggtctaccctaccagaagtgctgtctaaattagatttaaacaacatctcggcagcttcagtgatggcatccggcttaattaaaggacagataattgcagttgatataccgactatgacactagtattgatggtccaaataccaagtatatcccctttaagacaagcaaagataatagatctaacttctataacaattcacacaaatagtcaagaagtacaagctgtagtaccggctagggttcttgagattggttcagaaatattaggattcgacggatcagtgtgccaaatcacgaaagatacaatcttttgtccttataatgatgcttatgtattacccatccagcagaagagatgcctacaaggccaaacaagagattgcgtgttcaccccagttgctggcaccttcccccggagatttcttactacatatggtactatagtagctaattgtagagatttagtatgttcttgtctacgacctcctcaaataatttatcaacctgatgaaagtccagttacaatcatagacaaagacctgtgcacaacattgactttagactccatcactatagagatccagaagtccattaatagtacttttcgacgtgaagtagtattagaatctactcaagttagatctttgactcctcttgatttatcaactgatttaaatcaatacaatcaattgctcaagagtgctgaagatcacatccaacgatcaactgattactt

SEQ ID NO: 70 - DNA, 1411 bp, humanParainfluenzatype4 CCRI-15622
ttaccaatctagatataactcatttaatgaatctaggaacggtaccaactgcaattagatctctagtttactacacttatactaaaccatcttatctcactgtagacttgattcccaacttgaagaatctggaccaaaattgcaattactcaagcttaaactactacaataaaactgcactaagcttaattcaaccaattgcagataacatcaatcgccttacaaagcccatcacaagctcagaggttcaaagtcgtttctttggggcagtcataggtacaattgctcttggtgtagccaccgctgcacaggttacagcagcaatcggtctagcaaaggctcaagaaaatgcaaaacttatactaactctcaaaaaagctgcgacagaaacaaatgaggcagttcgagatcttgcaaactccaataaaattgttgtaaagatgatatcagcaattcaaaatcaaataaacactatcattcaacctgctatagaccagattaattgtcaaattaaagacctacaggttgccaacatcttaaatttgtacctaacagagataacgactgttttccacaatcaattgaccaatcccgcattagagtcaattagcatccaagctctcaaaagcctcttaggatctaccctaccagaagtgctgtctaaattagatttgaacaacatctcagcagcttcagtgatggcatccggcttaattaaaggacagataattgcagttgatataccgactatgacactagttttgatggtccaaataccgagtatatcccccttaagacaagcaaagataatagatctaacttctataacaattcacacaaatagtcaagaagtacaagctgtagtaccagctagggttcttgagattggttcagaaatattaggattcgatggatcagtgtgccaaatcacgaaagatacagtcttttgtccttataatgatgcttatgtattacccatccagcagaagagatgcctacaaggccaaacaagagattgcgtgttcaccccagttgctggcactttccctcggagatttcttactacatatggtactatagtagctaattgtagagatttagtatgttcttgtctacgacctcctcaaataatctatcaacctgatgagaatccagttacaatcatagacaaagacctgtgcacaacattgactctagactccatcactatagagatccagaagtccattaatagtacttttcgacgtgaagtagtattagaatccactcaagtcagatctttgactcctcttgacttatcaactgatttaaatcaatacaatcaattactcaagagtgctgaagatcacatccaacgatcaactgattacttaaactcaattaatcctagtatagtcaataataacgcaat

SEQ ID NO: 71 - DNA, 1417 bp, humanParainfluenzatype4 CCRI-15530
ccaattaccaatctggatataactcatttaatgaatctaggaacggtaccaactgcaattagatctctagtttactacacttatactaaaccatcttatctcactgtagacttgattcccaacttgaagaatctggaccaaaattgtaattactcaagcttaaactactacaacaaaactgcactaagcttaattcaaccaattgcagataacatcaatcgccttacaaagcccatcgcaagctcagaggttcaaagtcgtttctttggggcagtcataggtacaattgctcttggtgtagccaccgctgcacaagttacagcagcaatcggtttagcaaaggctcaagaaaatgcaaaacttatactaactctcaaaaaagctgcgacagaaacaaatgaggcagttcgagatcttgcaaactccaataaaattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagcctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccctttaaggcaagcaaagataatagatctaacttctataacaattcacacaaatagtcaagaagtacaagctgtagtaccagctagggttcttgagattggttcagaaatattaggattcgatggatcagtgtgccaaatcacgaaagatacaatcttttgtccttataatgatgcttatgtattacccatccagcagaaaagatgcctacaaggtcaaacaagagattgcgtgttcaccccagttgctggcactttccctcggagatttcttactacatacggtactatagtagctaattgtagagatttagtatgttcttgtctacgacctcctcaaataatctatcaacctgatgaaaatccagttacaatcatagacaaagacctgtgcacaacattgactctagactccatcactatagagatccagaagtccattaatagtacttttcgacgtgaagtagtattagaatctactcaggtcagatctttgactcctcttgatttatcaactgatttaaatcaatacaatcaattactcaagagtgctgaagatcacatccaacgatcaactgattacttaaactcaattaatcctagtatagtcaataataacgcaataa

SEQ ID NO: 72 - DNA, 1395 bp, humanParainfluenzatype4 CCRI-15836
tttaatgaatctaggaacggtaccaactgcaattagatctctagtttactacacttatactaaaccatcttatctcactgtagacttgattcccaacttgaagaatctggaccaaaattgtaattactcaagcttaaattactacaacaaaactgcactaagcttaattcaaccaattgcagataacatcaatcgccttacaaagcccatcgcaagctcagaggttcaaagtcgtttctttggggcagtcataggcacaattgctcttggtgtagccaccgctgcacaagttacagcagcaatcggtctagcaaaggctcaagaaaatgcaaaacttatactaactctcaaaaaagctgcgacagaaacaaatgaggcagttcgagatcttgcaaactccaataaaattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagcctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccctttaagacaagcaaagataatagatctaacttctataacaattcacacaaatagtcaagaagtacaagctgtagtaccagctagggttcttgagattggttcagaaatattaggattcgatggatcagtgtgccaaatcacgaaagatacaatcttttgtccttataatgatgcttatgtattacccatccagcagaagagatgcctacaaggtcaaacaagagattgcgtgttcaccccagttgctggcactttccctcggagatttcttactacatatggtactatagtagctaattgtagagatttagtatgttcttgtctacgacctcctcaaataatctatcaacctgatgaaaatccagttacaatcatagacaaagacctgtgcacaacattgactctagactccattactatagagatccagaagtccattaatagtacttttcgacgtgaagtagtattagaatctactcaggtcagatctttgactcctcttgatttatcaactgatttaaatcaatacaatcaattactcaagagtgctgaagatcacatccaacgatcaactgattacttaaactcaattaatcctagtatagtcaataataacgcaataataat

SEQ ID NO: 73 - DNA, 1414 bp, humanParainfluenzatype4 CCRI-15774
ttaccaatctggatataactcatttaatgaatctaggaacggtaccaactgcaattagatctctagtttactacacttatactaaaccatcttatctcactgtagacttgattcccaacttgaagaatctggaccaaaattgtaattactcaagcttaaattactacaacaaaactgcactaagcttaattcaaccaattgcagataacatcaatcgccttacaaagcccatcgcaagctcagaggttcaaagtcgtttctttggggcagtcataggcacaattgctcttggtgtagccaccgctgcacaagttacagcagcaatcggtctagcaaaggctcaagaaaatgcaaaacttatactaactctcaaaaaagctgcgacagaaacaaatgaggcagttcgagatcttgcaaactccaataagattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagcctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccccttaagacaagcaaagataatagatctaacttctataacaattcacacaaatagtcaagaagtacaagctgtagtaccagccagggttcttgagattggctcagaaacactaggattcgatggatcagtgtgccaaatcacgaaagatacaatcttttgtccttataatgatgcttatgtattacccatccagcagaagagatgcctacaaggtcaaacaagagattgcgtgttcaccccagttgctggcactttccctcggagatttcttactacatatggcactatagtagctaattgtagagatttagtatgttcttgtctacgacctcctcaaataatctatcaacctgatgaaaatccagttacaatcatagacaaagacttgtgcacaacattgactctagactccatcactatagagatccagaagtccattaatagtacttttcgacgtgaagtagtattagaatctactcaggtcagatctttgactcctcttgatttatcaaccgatttaaatcaatacaatcagttactcaagagtgctgaagatcacatccaacgatcaactgattacttaaactcaattaatcctagtatagtcaataataacgcaataat

SEQ ID NO: 74 - DNA, 1421 bp, Human Parainfluenza type 4 CCRI-15546
ccaattaccaatctggatataactcatttaatgaatctaggaacggtaccaaccgcaattagatctctagtttactacacttatactaaaccatcttatctcactgtagacttgattcccaacttgaagaatctggaccaaaattgtaattactcaagcttaaattactacaacaaaactgcactaagcttaattcaaccaattgcagataacatcaatcgccttacaaagcccatcgcaagctcagaggttcaaagtcgtttctttggggcagtcataggcacaattgctcttggtgtagccaccgctgcacaagttacagcagcaatcggtctagcaaaggctcaagaaaatgcaaaacttatactaactctcaaaaaagctgcgacagaaacaaatgaggcagttcgagatcttgcaaactccaataagattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagtctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccccttaagacaagcaaagataatagatctaacttctataacaattcacacaaatagtcaagaagtacaagctgtagtaccagctagggttcttgagattggttcagaaatattaggattcgatggatcagtgtgccaaatcacgaaagatacaatcttttgtccttataatgatgcttatgtattacccatccagcagaagagatgcctacaaggtcaaacaagagattgcgtgttcaccccagttgctggcactttccctcggagatttcttactacatatggtactatagtagctaattgtagagatttagtatgttcttgtctacgacctcctcaaataatctatcaacctgatgaaaatccagttacaatcatagacaaagacctgtgcacaacattgactctagactccatcactatagagatccagaagtccattaatagtacttttcgacgtgaagtagtattagaatctactcaggtcagatctttgactcctcttgatttatcaaccgatttaaatcaatacaatcagttactcaagagtgctgaagatcacatccaacgatcaactgattacttaaactcaattaatcctagtatagtcaataataacgcaataataat

SEQ ID NO: 75 - DNA, 1360 bp, humanParainfluenzatype4 CCRI-15697
ttaccaatctggatataactcatttaatgaatctaggaacggtaccaaccgcaattagatctctagtttactacacttatactaaaccatcttatctcactgtagacttgattcccaacttgaagaatctggaccaaaattgtaattactcaagcttaaattactacaacaaaactgcactaagcttaattcaaccaattgcagataacatcaatcgccttacaaagcccatcgcaagctcagaggttcaaagtcgtttctttggggcagtcataggcacaattgctcttggtgtagccaccgctgcacaagttacagcagcaatcggtctagcaaaggctcaagaaaatgcaaaacttatactaactctcaaaaaagctgcgacagaaacaaatgaggcagttcgagatcttgcaaactccaataagattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagcctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccccttaagacaagcaaagataatagatctaacttctataacaattcacacaaatagtcaagaagtacaagctgtagtaccagctagggttcttgagattggttcagaaatattaggattcgatggatcagtgtgccaaatcacgaaagatacaatcttttgtccttataatgatgcttatgtattacccatccagcagaagagatgcctacaaggtcaaacaagagattgcgtgttcaccccagttgctggcactttccctcggagatttcttactacatatggtactatagtagctaattgtagagatttagtatgttcttgtctacgacctcctcaaataatctatcaacctgatgaaaatccagttacaatcatagacaaagacctgtgcacaacattgactctagactccatcactatagagatccagaagtccattaatagtacttttcgacgtgaagtagtattagaatctactcaggtcagatctttgactcctcttgatttatcaaccgatttaaatcaatacaatcagttactcaagagtgctgaagatcacatccaacgatc

SEQ ID NO: 76 - DNA, 1344 bp, humanParainfluenzatype4 CCRI-15701
aactcatttaatgaatctaggaacggtaccaactgcaattagatctctagtttactacacttatactaaaccatcttatctcactgtagacttgattcccaacttgaagaatctggaccaaaattgtaattactcaagcttaaattactacaacaaaactgcactaagcttaattcaaccaattgcagataacatcaatcgccttacaaagcccatcgcaagctcagaggttcaaagtcgtttctttggggcagtcataggcacaattgctcttggtgtagccaccgctgcacaagttacagcagcaatcggtctagcaaaggctcaagaaaatgcaaaacttatactaactctcaaaaaagctgcgacagaaacaaatgaggcagttcgagatcttgcaaactccaataaaattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagcctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccctttaagacaagcaaagataatagatctaacttctataacaattcacacaaatagtcaagaagtacaagctgtagtaccagctagggttcttgagattggttcagaaatattaggattcgatggatcagtgtgccaaatcacgaaagatacaatcttttgtccttataatgatgcttatgtattacccatccagcagaagagatgcctacaaggtcaaacaagagattgcgtgttcaccccagttgctggcactttccctcggagatttcttactacatatggtactatagtagctaattgtagagatttagtatgttcttgtctacgacctcctcaaataatctatcaacctgatgaaaatccagttacaatcatagacaaagacctgtgcacaacattgactctagactccattactatagagatccagaagtccattaatagtacttttcgacgtgaagtagtattagaatctactcaggtcagatctttgactcctcttgatttatcaactgatttaaatcaatacaatcaattactcaagagtgctgaagatcacatccaacgatc

SEQ ID NO: 77 - DNA, 1676 bp, humanParainfluenzatype4CCRI-15836cloned
gtgattgtaatacgactcactatagggcgaattgggccctctagatgcatgctcgagcggccgccagtgtgatggatatctgcagaattcgcccttatgggtgtcaaaggtttatctttaattatgattgggttattaatctcaccaattaccaatctggatataactcatttaatgaatctaggaacggtaccaactgcaattagatctctagtttactacacttatactaaaccatcttatctcactgtagacttgattcccaacttgaagaatctggaccaaaattgtaattactcaagcttaaattactacaacaaaactgcactaagcttaattcaaccaattgcagataacatcaatcgccttacaaagcccatcgcaagctcagaggttcaaagtcgtttctttggggcagtcataggcacaattgctcttggtgtagccaccgctgcacaagttacagcagcaatcggtctagcgaaggctcaagaaaatgcaaaacttatactaactctcaaaaaagctgcgacagaaacaaatgaggcagttcgagatcttgcaaactccaataaaattgttgtgaagatgatatcagcaattcaaaaccaaataaacactatcattcaacctgctatagaccagattaattgtcaaattaaagacctacaggttgccaacatcttaaatttgtacctaacagagataacgactgttttccacaatcaattgaccaatcccgcattagagtcaattagcattcaagctctcaaaagcctcttaggatctaccctaccagaagtgctgtctaaattagatttaaacaacatctcggcagcttcagtgatggcatccggcttaattaaaggacaaataattgcagttgatataccgactatgacactagttttgatggtccaaataccgagtatatcccctttaagacaagcaaagataatagatctaacttctataacaattcacacaaatagtcaagaagtacaagctgtagtaccagctagggttcttgagattggttcagaaatattaggattcgatggatcagtgtgccaaatcacgaaagatacaatcttttgtccttataatgatgcttatgtattacccatccagcagaagagatgcctacaaggtcaaacaagagattgcgtgttcaccccagttgctggcactttccctcggagatttcttactacatatggtactatagtagctaattgtagagatttagtatgttcttgtctacgacctcctcaaataatctatcaacctgatgaaaatccagttacaatcatagacaaagacctgtgcacaacattgactctagactccattactatagagatccagaagtccattaatagtacttttcgacgtgaagtagtattagaatctactcaggtcagatctttgactcctcttgatttatcaactgatttaaatcaatacaatcaattactcaagagtgctgaagatcacatccaacgatcaactgattacttaaactcaattaatcctagtatagtcaataataacgcaataataatattgatcatactctgcatcttattaatattgacagttacaatctgcataattaagggcgaattccagcacactggcggccgttactagtggatccgagctcggtaccaagc

SEQ ID NO: 78 - DNA, 15 bp, Enterovirus
tagtcctccggcccc

SEQ ID NO: 79 - DNA, 17 bp, Enterovirus
cacggacacccaaagta

SEQ ID NO: 80 - DNA, 20 bp, HumanRespiratorySyncytialvirus
tgctaaaagaaatgggagag

SEQ ID NO: 81 - DNA, 23 bp, HumanRespiratorySyncytialvirus
cctgctgctaatttagttattac

SEQ ID NO: 82 - DNA, 22 bp, Humanmetapneumovirus
ctacaggcagcaaagcagaaag

SEQ ID NO: 83 - DNA, 22 bp, Humanmetapneumovirus
tgttggatgacctggcaatgac

SEQ ID NO: 84 - DNA, 22 bp, InfluenzaA
cttgatattgtggattcttgat

SEQ ID NO: 85 - DNA, 22 bp, InfluenzaA
gatactcttccctcatagactc

SEQ ID NO: 86 - DNA, 22 bp, HumanParainfluenzatype1
cgagataagacctgcaatagga

SEQ ID NO: 87 - DNA, 22 bp, HumanParainfluenzatype1
gataatgcctgcaaggtaagac

SEQ ID NO: 88 - DNA, 20 bp, HumanParainfluenzatype2
tgccattgctggagatcaac

SEQ ID NO: 89 - DNA, 17 bp, HumanParainfluenzatype2
gggggaagartaggtag

SEQ ID NO: 90 - DNA, 17 bp, HumanParainfluenzatype3
gagtggcaacctcagca

SEQ ID NO: 91 - DNA, 19 bp, HumanParainfluenzatype3
cttcacaacctaatctcgc

SEQ ID NO: 92 - DNA, 17 bp, HumanCoronavirusSARS
cccaacccataaggtgt

SEQ ID NO: 93 - DNA, 16 bp, HumanCoronavirusSARS
aatagagctcgcaccg

SEQ ID NO: 94 - DNA, 20 bp, Coronavirus229EandCoronavirusNL
ayattagtgcttttaaagcc

SEQ ID NO: 95 - DNA, 20 bp, Coronavirus229EandCoronavirusNL
tcatcmacaaaaacaccagc

SEQ ID NO: 96 - DNA, 22 bp, CoronavirusOC-43
gcagatttgccagcttatatga

SEQ ID NO: 97 - DNA, 17 bp, CoronavirusOC-43
ctcaacaatgcggtgtc

SEQ ID NO: 98 - DNA, 17 bp, HumanAdenovirus
ccgggtctggtgcagtt

SEQ ID NO: 99 - DNA, 18 bp, humanAdenovirus
  misc_feature (3)..(3): n is in place of I for Inosine
acngcscgratgtcaaag

SEQ ID NO: 100 - DNA, 20 bp, InfluenzaB
tggagaaggcaaagcagaac

SEQ ID NO: 101 - DNA, 20 bp, InfluenzaB
atagakgcaccaattagtgc

SEQ ID NO: 102 - DNA, 19 bp, humanParainfluenzatype4
  misc_feature (2)..(2): n is in place of i for Inosine
gntcagtgtgccaaatcac

SEQ ID NO: 103 - DNA, 17 bp, HumanParainfluenzatype4
ggagtgaacacgcaatc

SEQ ID NO: 104 - DNA, 23 bp, Artificial Sequence
  The sequence is a completely synthesized primer
cactcccctgtgaggaactactg

SEQ ID NO: 105 - DNA, 24 bp, Artificial Sequence
  The sequence is a completely synthesized primer
aggctgcacgacactcatactaac

SEQ ID NO: 106 - DNA, 20 bp, humanEnterovirus
ttgggattagccgcattcag

SEQ ID NO: 107 - DNA, 20 bp, humanEnterovirus
ttaaggttagccacattcag

SEQ ID NO: 108 - DNA, 20 bp, humanEnterovirus
ttagggttagccacattcag

SEQ ID NO: 109 - DNA, 20 bp, humanEnterovirus
ttaggattagccacattcag

SEQ ID NO: 110 - DNA, 20 bp, humanEnterovirus
tgaggattagccacattcag

SEQ ID NO: 111 - DNA, 20 bp, humanEnterovirus
ttagggttagccgcattcag

SEQ ID NO: 112 - DNA, 20 bp, humanEnterovirus
gtttaggttagccgcattcg

SEQ ID NO: 113 - DNA, 20 bp, humanEnterovirus
ttaggattagccgcattcag

SEQ ID NO: 114 - DNA, 20 bp, humanEnterovirus
ttaaggttagccgcattcag

SEQ ID NO: 115 - DNA, 20 bp, humanEnterovirus
ttaggattatccgcattcag

SEQ ID NO: 116 - DNA, 20 bp, humanEnterovirus
tttaggttagccgcattcgg

SEQ ID NO: 117 - DNA, 20 bp, humanEnterovirus
gttggattacccgcattcaa

SEQ ID NO: 118 - DNA, 20 bp, humanEnterovirus
gttggattagccgcattcag

SEQ ID NO: 119 - DNA, 20 bp, humanEnterovirus
cagttagggttagccgcatt

SEQ ID NO: 120 - DNA, 20 bp, humanEnterovirus
cagttaggattatccgcatt

SEQ ID NO: 121 - DNA, 20 bp, humanRespiratorySyncytialvirus
catgcctatattctggagcc

SEQ ID NO: 122 - DNA, 20 bp, humanRespiratorySyncytialvirus
catgcctgtattctggagct

SEQ ID NO: 123 - DNA, 20 bp, humanRespiratorySyncytialvirus
catgcctgtattctggagcc

SEQ ID NO: 124 - DNA, 20 bp, humanRespiratorySyncytialvirus
catgcctatattctggagcc

SEQ ID NO: 125 - DNA, 20 bp, humanRespiratorySyncytialvirus
catgcctgtattctggagct

SEQ ID NO: 126 - DNA, 20 bp, humanRespiratorySyncytialvirus
catgcctgtattctggagcc

SEQ ID NO: 127 - DNA, 20 bp, humanRespiratorySyncytialvirus
catgcctgtattccggtgcc

SEQ ID NO: 128 - DNA, 20 bp, humanMetapneumovirus
gcaagcttatggggccggtc

SEQ ID NO: 129 - DNA, 20 bp, humanMetapneumovirus
gcaagcttatggagccggtc

SEQ ID NO: 130 - DNA, 20 bp, humanMetapneumovirus
gcaagcttacggtgctggtc

SEQ ID NO: 131 - DNA, 20 bp, humanMetapneumovirus
gcaagcttatggtgctggtc

SEQ ID NO: 132 - DNA, 20 bp, humanMetapneumovirus
gcaagcttatggagctggcc

SEQ ID NO: 133 - DNA, 20 bp, humanMetapneumovirus
gcaagcttatggagctggtc

SEQ ID NO: 134 - DNA, 20 bp, humanInfluenzaA
catttgaaaaaaagacgatc

SEQ ID NO: 135 - DNA, 20 bp, humanInfluenzaA
cttttggaaaaaaggcgatc

SEQ ID NO: 136 - DNA, 20 bp, humanInfluenzaA
catttgaagaaaagacgatc

SEQ ID NO: 137 - DNA, 20 bp, humanInfluenzaA
catttgaaaaaaaggcgatc

SEQ ID NO: 138 - DNA, 20 bp, humanInfluenzaA
catttgaagaaaaggcgatc

SEQ ID NO: 139 - DNA, 20 bp, humanInfluenzaA
tatttgaagaaaagacgatc

SEQ ID NO: 140 - DNA, 20 bp, humanInfluenzaA
cttttgggaaaaaggcgatc

SEQ ID NO: 141 - DNA, 20 bp, humanInfluenzaA
aatttgaaaaaaaggcgatc

SEQ ID NO: 142 - DNA, 20 bp, humanInfluenzaA
cttttggaaaaaagccgatc

SEQ ID NO: 143 - DNA, 20 bp, humanInfluenzaA
aatttgaaaaaaagacgatc

SEQ ID NO: 144 - DNA, 20 bp, humanInfluenzaA
catttgaaaaaaagatgatc

SEQ ID NO: 145 - DNA, 20 bp, humanInfluenzaA
gaaaagagggccttctacgg

SEQ ID NO: 146 - DNA, 20 bp, humanInfluenzaA
gaaaagagggcctcctacgg

SEQ ID NO: 147 - DNA, 20 bp, HumanInfluenzaA
gaaaagagggcctcctacgg

SEQ ID NO: 148 - DNA, 20 bp, humanInfluenzaA
tagaagaggcccttctacgg

SEQ ID NO: 149 - DNA, 20 bp, humanInfluenzaA
gaaaggagggccttctacgg

SEQ ID NO: 150 - DNA, 20 bp, humanInfluenzaA
aaaaagagggccttctacgg

SEQ ID NO: 151 - DNA, 20 bp, humanInfluenzaA
taaaagaggcccttctacgg

SEQ ID NO: 152 - DNA, 20 bp, humanInfluenzaA
gaaaagagggcctgctacgg

SEQ ID NO: 153 - DNA, 20 bp, humanParainfluenzatype1
gccgtagtctcacaccttag

SEQ ID NO: 154 - DNA, 20 bp, humanParainfluenzatype1
gccgtggtttcacaccttag

SEQ ID NO: 155 - DNA, 20 bp, humanParainfluenzatype2
gattgaatgacccctacatt

SEQ ID NO: 156 - DNA, 20 bp, humanParainfluenzatype2
gattgaatgacccctatatt

SEQ ID NO: 157 - DNA, 20 bp, humanParainfluenzatype2
gattgaatgacccctatgtt

SEQ ID NO: 158 - DNA, 20 bp, humanParainfluenzatype3
cagagcaactgccgctgtaa

SEQ ID NO: 159 - DNA, 20 bp, humanCoronavirusSARS
gctggtgtctctatctgtag

SEQ ID NO: 160 - DNA, 20 bp, humanCoronavirusSARS
ctacagatagagacaccagc

SEQ ID NO: 161 - DNA, 20 bp, humanCoronavirus229E
ccatatccagatcctagccg

SEQ ID NO: 162 - DNA, 20 bp, humanCoronavirusNL
gccttacccagatcctagta

SEQ ID NO: 163 - DNA, 20 bp, humanCoronavirusOC-43
aggtgtgtaaccttagcaac

SEQ ID NO: 164 - DNA, 20 bp, humanCoronavirusOC-43
aaccacggttatatgtgcac

SEQ ID NO: 165 - DNA, 20 bp, humanAdenovirus
ttatttcccagattgaagta

SEQ ID NO: 166 - DNA, 20 bp, humanAdenovirus
ttgttccccagattgaagta

SEQ ID NO: 167 - DNA, 20 bp, humanAdenovirus
ttgttccccatactgaagta

SEQ ID NO: 168 - DNA, 20 bp, humanAdenovirus
ttgttattcaggctgaagta

SEQ ID NO: 169 - DNA, 20 bp, HumanAdenovirus
ttgttccccatactgaagta

SEQ ID NO: 170 - DNA, 20 bp, humanAdenovirus
ttgttccccagactgaagta

SEQ ID NO: 171 - DNA, 20 bp, humanAdenovirus
ttgttccccaaactgaagta

SEQ ID NO: 172 - DNA, 20 bp, humanAdenovirus
gctacggtgggatttctaaa

SEQ ID NO: 173 - DNA, 20 bp, humanAdenovirus
gccacggtggggttcctaaa

SEQ ID NO: 174 - DNA, 20 bp, humanAdenovirus
gccactgtggggtttctaaa

SEQ ID NO: 175 - DNA, 20 bp, humanAdenovirus
gccaccgtggggttcctaaa

SEQ ID NO: 176 - DNA, 20 bp, humanAdenovirus
gccaccgtggggtttctaaa

SEQ ID NO: 177 - DNA, 20 bp, humanAdenovirus
ttgttccccatactgaagta

SEQ ID NO: 178 - DNA, 20 bp, humanAdenovirus
gccactgtggggtttctaaa

SEQ ID NO: 179 - DNA, 20 bp, humanInfluenzaB
aaccaacagtgtaatttttc

SEQ ID NO: 180 - DNA, 20 bp, humanInfluenzaB
aaccaacagtgtagtttttc

SEQ ID NO: 181 - DNA, 20 bp, humanInfluenzaB
aaccaacagtgcagtttttc

SEQ ID NO: 182 - DNA, 20 bp, humanInfluenzaB
aaccagcagtgcagtttttc

SEQ ID NO: 183 - DNA, 20 bp, humanInfluenzaB
aaccagcaatgcagtttttc

SEQ ID NO: 184 - DNA, 20 bp, Parainfluenzatype4
tataaggacaaaagattgta

SEQ ID NO: 185 - DNA, 20 bp, Parainfluenzatype4
tataaggacaaaagactgta

SEQ ID NO: 186 - DNA, 20 bp, Parainfluenzatype4
tgtagggacaaaagactgta

SEQ ID NO: 187 - DNA, 20 bp, Parainfluenzatype4
cttctgctggatgggtaata

SEQ ID NO: 188 - DNA, 20 bp, Parainfluenzatype4
tttctgctggatgggtaata

SEQ ID NO: 189 - DNA, 20 bp, Artificial Sequence
  Internal Control Multiplex 2
ccggtgcagccttttttctc

SEQ ID NO: 190 - DNA, 20 bp, Artificial Sequence
  Internal Control Multiplex 4
gctcaggctctccccgtgga

SEQ ID NO: 191 - DNA, 20 bp, Artificial Sequence
  Internal Control Multiplex 3
gtattaacgaagcgctaacc

SEQ ID NO: 192 - DNA, 20 bp, Artificial Sequence
  Internal Control Multiplex 1
tgcgcacggcgggcgtgatc

SEQ ID NO: 193 - DNA, 20 bp, Artificial Sequence
  Cell Lysis Control
tagacgctttctgcgtgaag

SEQ ID NO: 194 - DNA, 20 bp, humanEnterovirus
ttaggattggccgcattcag

SEQ ID NO: 195 - DNA, 20 bp, humanEnterovirus
ttgggaatagccgcattcag

SEQ ID NO: 196 - DNA, 20 bp, humanEnterovirus
gttaggattagcgcattcag

SEQ ID NO: 197 - DNA, 20 bp, humanRespiratorySyncytialvirus
tgtgggatgataatactgtg

SEQ ID NO: 198 - DNA, 20 bp, humanRespiratorySyncytialvirus
tgtgggatgataatattatg

SEQ ID NO: 199 - DNA, 20 bp, humanRespiratorySyncytialvirus
tgtggaatgataatactgtg

SEQ ID NO: 200 - DNA, 20 bp, humanRespiratorySyncytialVirus
tgtgggatgataatactatg

SEQ ID NO: 201 - DNA, 20 bp, humanInfluenzaA
aatttgaagaaaagacgatc

SEQ ID NO: 202 - DNA, 20 bp, humanInfluenzaA
aacttgaagaaaagacgatc

SEQ ID NO: 203 - DNA, 20 bp, humanInfluenzaA
cttttggaaaaaaggcgatc

SEQ ID NO: 204 - DNA, 20 bp, humanInfluenzaA
cttttggagaaaaggcgatc

SEQ ID NO: 205 - DNA, 21 bp, humanInfluenzaA
aaaccgtatttaaggcgacga

SEQ ID NO: 206 - DNA, 21 bp, humanInfluenzaA
aaaccgtatttaaggcggcga

SEQ ID NO: 207 - DNA, 21 bp, humanInfluenzaA
aaatcgtatttaaggcgacga

SEQ ID NO: 208 - DNA, 21 bp, humanInfluenzaA
aacccgtatttaaggcgacga

SEQ ID NO: 209 - DNA, 20 bp, humanParainfluenzatype3
tcacagcggcggttgctctg

SEQ ID NO: 210 - DNA, 20 bp, humanInfluenzaB
aaccaacaatgtaatttttc

SEQ ID NO: 211 - DNA, 20 bp, humanInfluenzaB
aaccagcagtgtaatttttc

SEQ ID NO: 212 - DNA, 20 bp, humanInfluenzaB
aaccaacagtgcaatttttc

SEQ ID NO: 213 - DNA, 20 bp, humanInfluenzaB
aaccaccagtgtaatttttc

SEQ ID NO: 214 - DNA, 20 bp, humanInfluenzaB
aaccacagtggtaatttttc

SEQ ID NO: 215 - DNA, 30 bp, Artificial Sequence
  Internal Control Multiplex 3
cccggcgccgtttcggcaaaaaggtatccg

SEQ ID NO: 216 - DNA, 30 bp, Artificial Sequence
  Internal Control Multiplex 2
acgactgcgtccggtgcagccttttttctc

SEQ ID NO: 217 - DNA, 30 bp, Artificial Sequence
  Internal Control Multiplex 2
ctcttttttccgacgtggccacgcagtcgt

SEQ ID NO: 218 - DNA, 30 bp, Artificial Sequence
  Internal Control Multiplex 4
tccggtcaaagctcaggctctccccgtgga

SEQ ID NO: 219 - DNA, 30 bp, Artificial Sequence
  Internal Control Multiplex 4
aggtgcccctctcggactcgtttgaccgga

SEQ ID NO: 220 - DNA, 30 bp, Artificial Sequence
  Internal Control Multiplex 4
cgccccgccctgccactcatcgcagtactg

SEQ ID NO: 221 - DNA, 30 bp, Artificial Sequence
  Internal Control Multiplex 3
ccacgccgaaacaagcgccctgcaccatta

SEQ ID NO: 222 - DNA, 30 bp, Artificial Sequence
  Internal Control Multiplex 3
aacagtccgcaccgctgccggtagctcctt

SEQ ID NO: 223 - DNA, 30 bp, Artificial Sequence
  Internal Control Multiplex 1
gttctccgggttcggcttcaggaccacctg

SEQ ID NO: 224 - DNA, 30 bp, Artificial Sequence
  Internal Control Multiplex 1
gaaggtgcgcacggcgggcgtgatcggtcc

SEQ ID NO: 225 - DNA, 20 bp, humanParainfluenzatype3
cagagcaactgctgctgtaa

SEQ ID NO: 226 - DNA, 20 bp, humanParainfluenzatype3
caaagcaactgccgctgtaa

SEQ ID NO: 227 - DNA, 20 bp, humanParainfluenzatype3
cggagcgactgctgctgtga

SEQ ID NO: 228 - DNA, 20 bp, humanParainfluenzatype3
tagagcaactgctgctgtaa

SEQ ID NO: 229 - DNA, 20 bp, humanParainfluenzatype3
gatcttgcctgcttggcttc